(19) United States
(12) Patent Application Publication
(10) Pub. No.: US 2022/0339281 A1
(43) Pub. Date: Oct. 27, 2022

(54) HEPATITIS B IMMUNISATION REGIMEN AND COMPOSITIONS

(71) Applicants: GalxoSmithKline Biologicals SA, Rixensart (BE); GalxoSmithKline Intellectual Property Development Limited, Brentford, Middlesex (GB)

(72) Inventors: Babak BAYAT, Rixensart (BE); Robert Kiyoshi HAMATAKE, Research Triagle Park, NC (US); Clarisse Marie Madeleine LORIN, Rixensart (BE); Ventzislav Bojidarov VASSILEV, Rixensart (BE); Lucile Eve-Renee WARTER, Rixensart (BE); Shihy Kieffer YOU, College Park (PA)

(21) Appl. No.: 17/436,183

(22) PCT Filed: Mar. 4, 2020
(86) PCT No.: PCT/EP2020/055755

§ 371 Date: Sep. 3, 2021

Related U.S. Application Data
(60) Provisional application No. 62/814,261, filed on Mar. 5, 2019.

(51) Int. Cl. A61K 39/29; C12N 15/86

(52) U.S. Cl. CPC A61K 39/292; C12N 15/86; A61K 2039/53

(57) ABSTRACT

There is provided a method of treating chronic hepatitis B infection (CHB) and/or chronic hepatitis D infection (CHD) in a human, comprising the steps of:
a) administering to the human a composition comprising an antisense oligonucleotide (ASO) 10 to 30 nucleosides in length, targeted to a HBV nucleic acid (an HBV ASO);
b) administering to the human a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) administering to the human a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) administering to the human a composition comprising a recombinant hepatitis B surface antigen (HBs), recombinant hepatitis B virus core antigen (HBc) and an adjuvant.

### FIELD OF THE INVENTION

The present invention relates to immunisation regimens which are particularly suited for the treatment of chronic hepatitis B, to methods for the treatment of chronic hepatitis B and to compositions for use in such regimens and methods. Said regimens and methods involve the administration of compositions comprising antisense oligonucleotides, compositions comprising vectors delivering hepatitis B antigens and compositions comprising recombinant hepatitis B antigen proteins.

### BACKGROUND TO THE INVENTION

The hepatitis B virus is a DNA virus with a partially double stranded circular DNA genome, the full length strand of which is 3020-3320 nucleotides long and the shorter strand is 1700-2800 nucleotides long. The viral DNA is found in the cell nucleus soon after infection of the cell. After infection, cellular DNA polymerases render the viral genome fully double stranded and the ends are joined. The viral core (C), surface (S) and X genes each overlap with the viral polymerase (P) gene in the genome. The hepatitis B core antigen (HBcAg), pre-core and HBeAg are produced by differential processing from one gene which has two separate start codons. Similarly, the surface gene has three start codons and produces three proteins of different lengths, the large (pre-S1+pre-S2+S), middle (pre-S2+S) and small (S) surface antigens. Hepatitis B virus (HBV) infection is a major public health problem. Globally, approximately 257 million people are infected with HBV [WHO, 2017]. The clinical course and outcome of HBV infection is largely driven by the age at infection and a complex interaction between the virus and the host immune response [Ott, 2012; Maini, 2016]. Thus, exposure to HBV may lead to acute hepatitis that resolves spontaneously or may progress to various forms of chronic infection, including the inactive hepatitis B surface antigen (HBsAg) carrier state, chronic hepatitis, cirrhosis and hepatocellular carcinoma (HCC) [Liaw, 2009]. The prevalence of HBsAg in the adult population is >2%, with rates of 5-8% in South East Asia and China and >8% in the African Region. Between 15-40% of persons with chronic hepatitis B infection (defined as serum HBsAg being detected for more than 6 months) will develop liver sequelae, of which liver cirrhosis (LC), hepatic decompensation and HCC are the major complications.

Although implementation of universal prophylactic hepatitis B immunization in infants has been highly effective in reducing the incidence and prevalence of hepatitis B in many endemic countries, it has not yet led to a strong decrease in the prevalence of chronic hepatitis B infection (CHB) in adolescents and adults, and it is not expected to impact on HBV-related deaths until several decades after introduction. In 2015, hepatitis B accounted for 887,000 deaths, mostly from liver cirrhosis and HCC [WHO, 2017].

Clinical management of chronic hepatitis B aims to improve survival and quality of life by preventing disease progression, and consequently HCC development [Liaw, 2013]. Current treatment strategy is mainly based on the long-term suppression of HBV DNA replication to achieve the stabilisation of HBV-induced liver disease and to prevent progression. Serum HBV DNA level is a cornerstone endpoint of all current treatment modalities. Achieving loss of (detectable) hepatitis B e-antigen (HBeAg) is another valuable biomarker, however HBsAg loss, with or without anti-HBs seroconversion, is generally considered an optimal endpoint representing “functional cure”, as it indicates profound suppression of HBV replication and viral protein expression [Block, 2017; Cornberg, 2017]. Currently, there are two main treatment options for CHB patients: either by pegylated interferon alpha (PegIFNα) or by nucleo(s/t)ide analogues (NA) [EASL, 2017]. PegIFNα aiming at induction of a long-term immune control with a finite duration treatment may achieve sustained off-treatment control, but durable virological response and hepatitis B surface antigen (HBsAg) loss is limited to a small proportion of patients. In addition, owing to its poor tolerability and long-term safety concerns, a significant number of patients are ineligible for this type of treatment.

NAs act by suppressing DNA replication through inhibition of HBV polymerase reverse transcriptase activity. The NAs approved in Europe for HBV treatment include entecavir (ETV), tenofovir disoproxil fumarate (TDF) and tenofovir alafenamide (TAF) that are associated with high barrier against HBV resistance as well as lamivudine (LAM), adefovir dipivoxil (ADV) and telbivudine (TBV) that are associated with low barrier to HBV resistance. The main advantage of treatment with a potent NA with high barrier to resistance is its predictable high long-term antiviral efficacy leading to HBV DNA suppression in the vast majority of compliant patients as well as its favourable safety profile. The disadvantage of NA treatment is its long-term therapeutic regimen, because a NA does not usually achieve HBV eradication and NA discontinuation may lead to HBV relapse [Kranidioti, 2015]. HBsAg loss representing a functional cure is now the gold standard treatment endpoint in CHB [Block, 2017; Cornberg, 2017], which however, is rarely achieved with NA treatment [Zoutendijk, 2011].

Because of a low rate of HBsAg seroclearance [Zoutendijk, 2011] and a high risk of off-NA viral relapse [Kranidioti, 2015], most patients are maintained under long-term or even indefinite NA therapy, which could be associated with reduction in patient compliance to therapy, increase in financial costs and increased risk for drug toxicity and drug resistance mutations upon long-term exposure [Terrault, 2015]. A new strategy is therefore necessary to supplement to the NA therapy to achieve “functional cure” with a finite regimen.

Antisense therapy differs from nucleoside therapy in that it can directly target the RNA transcripts for the antigens and thereby reduce serum HBeAg and HBsAg levels. In addition to antisense therapies and novel antiviral drugs, new treatment strategies currently being explored include immunotherapeutic strategies that boost HBV-specific adaptive immune response or activate innate intrahepatic immunity [Durantel, 2016]. So far, none of these experimental treatments have been shown to be efficacious. Among the vaccination strategies evaluated, none was able to induce a robust poly-functional CD8⁺ T-cell response to HBV core antigen (HBcAg) that is of key importance to restore immune control on the virus [Lau, 2002; Li, 2011; Liang, 2011; Bertoletti, 2012; Boni, 2012]. Early efforts on recombinant vaccines based on HBV surface and/or PreS antigens preliminarily induced antibody responses but no HBV-specific CD8+ T-cell response, with no clinical or virological benefit [Jung, 2002; Vandepapelière, 2007]. A DNA vaccine expressing HBV envelope failed to restore T cell response specific to HBsAg and HBcAg thus did not decrease the risk of relapse in patients after NA discontinuation [Fontaine, 2015]. With new delivery systems, a DNA vaccine (prime vaccine) and MVA viral vector vaccine (boost vaccine) encoding S, preS1/S2 showed no T cell induction or reduction in viremia suggesting HBV PreS and surface antigens alone are not sufficient to cure patients [Cavenaugh, 2011]. More recently, vaccine strategies targeting multiple HBV antigens and new delivery systems have been investigated. A recombinant HBsAg/HBcAg vaccine led to a viral load decrease to a very low level (i.e. ˜50 IU/ml) in only half of the patients [Al-Mahtab, 2013]. A DNA vaccine encoding S, preS1/S2, core, polymerase and X proteins with genetically adjuvanted IL-12 together with lamivudine induced a multi-specific T cell response and a >2 log 10 decrease in viral load in half of the patients. However, changes in quantitative detection of HBsAg, loss of HBsAg or HBsAg seroconversion were not observed in any patients [Yang, 2012]. The GS-4774 vaccine, a yeast-based T cell vaccine expressing large S, core and X proteins of HBV did not provide significant reduction in HBsAg in virally-suppressed CHB patients [Lok, 2016].

There remains an unmet need for a treatment for chronic hepatitis B which can clear HBsAg in order to allow patients to safely discontinue NA therapy without virological or clinical relapse.

Hepatitis D virus (HDV) (also known a hepatitis delta) is a virus that requires hepatitis B virus for its replication. HDV infection occurs simultaneously or as a super-infection with HBV. HDV is transmitted through contact with blood or other bodily fluids of an infected individual, Vertical transmission from mother to child is rare. At least 5% of people with chronic HBV are co-infected with HDV, however this is likely an underestimation, as many countries do not report the prevalence of HDV. Hepatitis D infection can be prevented by hepatitis B vaccination, and since the introduction of successful national HBV prophylactic vaccination campaigns in the 1980s, the number of HDV infections has also decreased. HBV-HDV co-infection is considered the most severe form of chronic viral hepatitis due to more rapid progression toward liver-related death and hepatocellular carcinoma. Treatment is via administration of Pegylated interferon, but the rate of sustained virological response is low [WHO 2018]. Currently, treatment rates are also low. There remains an unmet need for a treatment which can halt progression of, or reverse, chronic hepatitis caused by HDV, and/or can clear chronic HDV infection (chronic hepatitis D—CHD) or HBV/HDV co-infection (CHB/CHD).

### SUMMARY OF THE INVENTION

In one aspect, there is provided a method of treating chronic hepatitis B infection (CHB) and/or chronic hepatitis D infection (CHD) in a human, comprising the steps of:
a) administering to the human a composition comprising an antisense oligonucleotide (ASO) 10 to 30 nucleosides in length, targeted to a HBV nucleic acid (an HBV ASO);
b) administering to the human a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) administering to the human a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) administering to the human a composition comprising a recombinant hepatitis B surface antigen (HBs), a recombinant hepatitis B virus core antigen (HBc) and an adjuvant.

In one embodiment, the steps of the method are carried out sequentially, with step b) preceding step c) and step c) preceding step d). Optionally step a) may be repeated. Optionally, step d) may be repeated. In another embodiment, step d) is carried out concomitantly with step b) and/or with step c).

In one specific embodiment, step a) is repeated and then stopped, after which step b), step c), and step d) are carried out sequentially. Optionally, step d) may be repeated. In another embodiment, step a) is repeated and then stopped before any subsequent steps, and step d) is carried out concomitantly with step b) and/or with step c). In these embodiments, the ASO of step a) is administered before the other compositions.

Thus, in another aspect, there is provided a method of treating chronic hepatitis B infection (CHB) and/or chronic hepatitis D infection (CHD) in a human, comprising the steps of:
a) administering to the human a composition comprising an antisense oligonucleotide (ASO) 10 to 30 nucleosides in length, targeted to a HBV nucleic acid (an HBV ASO);
b) administering to the human i) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc) and, concomitantly, ii) a composition comprising a recombinant hepatitis B surface antigen (HBs), a recombinant hepatitis B virus core antigen (HBc) and an adjuvant; and
c) administering to the human i) a composition comprising a Modified Vaccinia Virus Ankara (MVA) vector comprising a polynucleotide encoding a hepatitis B surface antigen (HBs) and a nucleic acid encoding a hepatitis B virus core antigen (HBc) and, concomitantly, a composition comprising a recombinant hepatitis B surface antigen (HBs), a recombinant hepatitis B virus core antigen (HBc) and an adjuvant.

In one embodiment, the steps of the method are carried out sequentially, with step a) preceding step b) and step b) preceding step c). Optionally step a) may be repeated. Optionally, step c) may be repeated.

In one specific embodiment, step a) is repeated and then stopped, after which step b) and step c) are carried out sequentially. Optionally, step c) may be repeated. In these embodiments, the ASO of step a) is administered before the other compositions.

In another aspect, there is provided an immunogenic combination for use in a method of treating chronic hepatitis B infection (CHB) and/or chronic hepatitis D infection (CHD) in a human, the immunogenic combination comprising:
a) a composition comprising an antisense oligonucleotide (ASO) 10 to 30 nucleosides in length, targeted to a HBV nucleic acid (an HBV ASO);
b) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) a composition comprising a recombinant hepatitis B surface antigen (HBs), a recombinant hepatitis B virus core antigen (HBc) and an adjuvant,
wherein the method comprises administering the compositions sequentially or concomitantly to the human.

In another aspect, there is provided an immunogenic composition for use in a method of treating chronic hepatitis B infection (CHB) and/or chronic hepatitis D infection (CHD) in a human, the immunogenic composition comprising a composition comprising an antisense oligonucleotide (ASO) 10 to 30 nucleosides in length, targeted to a HBV nucleic acid (an HBV ASO) and a replication-defective chimpanzee adenoviral (ChAd) vector comprising a polynucleotide encoding a hepatitis B surface antigen (HBs), a nucleic acid encoding a hepatitis B virus core antigen (HBc) and a nucleic acid encoding the human invariant chain (hIi) fused to the HBc, wherein the method comprises administration of the composition in a prime-boost regimen with at least one other immunogenic composition. In certain embodiments, the immunogenic composition for use in a method of treating chronic CHB and/or CHD further comprises one or more recombinant HBV protein antigens.

In a further aspect, there is provided an immunogenic composition for use in a method of treating chronic hepatitis B infection (CHB) and/or chronic hepatitis D infection (CHD) in a human, the immunogenic composition comprising a composition comprising an antisense oligonucleotide (ASO) 10 to 30 nucleosides in length, targeted to a HBV nucleic acid (an HBV ASO) and a Modified Vaccinia Virus Ankara (MVA) vector comprising a polynucleotide encoding a hepatitis B surface antigen (HBs) and a nucleic acid encoding a hepatitis B virus core antigen (HBc) wherein the method comprises administration of the composition in a prime-boost regimen with at least one other immunogenic composition. In certain embodiments, the immunogenic composition for use in a method of treating chronic CHB and/or CHD further comprises one or more recombinant HBV protein antigens.

In a further aspect, there is provided an immunogenic composition for use in a method of treating chronic hepatitis B infection (CHB) and/or chronic hepatitis D infection (CHD) in a human, the immunogenic composition comprising a composition comprising an antisense oligonucleotide 10 to 30 nucleosides in length, targeted to a HBV nucleic acid (an HBV ASO), a recombinant hepatitis B surface antigen (HBs), a C-terminal truncated recombinant hepatitis B virus core antigen (HBc) and an adjuvant containing MPL (3-D Monophosphoryl lipid A) and QS-21 (a triterpene glycoside purified from the bark of Quillaja saponaria), wherein the method comprises administration of the composition in a prime-boost regimen with at least one other immunogenic composition. In certain embodiments, the immunogenic composition for use in a method of treating chronic CHB and/or CHD further comprises one or more vectors encoding one or more HBV antigens.

In a further aspect, there is provided an immunogenic combination comprising:
a) a composition comprising an antisense oligonucleotide (ASO) 10 to 30 nucleosides in length, targeted to a HBV nucleic acid (an HBV ASO);
b) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) a composition comprising a recombinant hepatitis B surface antigen (HBs), recombinant hepatitis B virus core antigen (HBc) and an adjuvant.

The immunogenic combination may find use in a method for treating chronic hepatitis B (CBH) by administration of the compositions in a prime-boost regimen.

The immunogenic combination may find use in a method for treating CHB and/or CHD in a human by administration of the compositions sequentially or concomitantly.

In another aspect, there is provided a method of treating chronic hepatitis B infection (CHB) and/or chronic hepatitis D infection (CHD) in a human, comprising the steps of:
a) administering to the human a composition comprising an antisense oligonucleotide (ASO) 10 to 30 nucleosides in length, targeted to a HBV nucleic acid (an HBV ASO);
b) administering to the human a composition comprising a replication-defective chimpanzee adenoviral (ChAd) vector comprising a polynucleotide encoding a hepatitis B surface antigen (HBs) and a nucleic acid encoding a hepatitis B virus core antigen (HBc); and
c) administering to the human a composition comprising a recombinant hepatitis B surface antigen (HBs), a recombinant hepatitis B virus core antigen (HBc) and an adjuvant.

In one embodiment, the steps of the method are carried out sequentially, with step a) preceding step b) and step b) preceding step c). Optionally step a) may be repeated. Optionally, step c) may be repeated. In another embodiment, step c) is carried out concomitantly with step b).

In one specific embodiment, step a) is repeated and then stopped, after which step b) and step c) are carried out sequentially. Optionally, step c) may be repeated. In another embodiment, step c) is carried out concomitantly with step b). In these embodiments, the ASO of step a) is administered before the other compositions.

Thus, in another aspect, there is provided a method of treating chronic hepatitis B infection (CHB) and/or chronic hepatitis D infection (CHD) in a human, comprising the steps of:
a) administering to the human a composition comprising an antisense oligonucleotide (ASO) 10 to 30 nucleosides in length, targeted to a HBV nucleic acid (an HBV ASO); and
b) administering to the human i) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc) and, concomitantly, ii) a composition comprising a recombinant hepatitis B surface antigen (HBs), a recombinant hepatitis B virus core antigen (HBc) and an adjuvant.

In one embodiment, the steps of the method are carried out sequentially, with step a) preceding step b). Optionally step a) may be repeated. Optionally, step b) may be repeated.

In another aspect, there is provided an immunogenic combination for use in a method of treating chronic hepatitis B infection (CHB) and/or chronic hepatitis D infection (CHD) in a human, the immunogenic combination comprising:
a) a composition comprising an antisense oligonucleotide (ASO) 10 to 30 nucleosides in length, targeted to a HBV nucleic acid (an HBV ASO);
b) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc); and
c) a composition comprising a recombinant hepatitis B surface antigen (HBs), a recombinant hepatitis B virus core antigen (HBc) and an adjuvant,
wherein the method comprises administering the compositions sequentially or concomitantly to the human.

In a further aspect, there is provided an immunogenic combination comprising:
a) a composition comprising an antisense oligonucleotide (ASO) 10 to 30 nucleosides in length, targeted to a HBV nucleic acid (an HBV ASO);
b) a composition comprising a replication-defective chimpanzee adenoviral (ChAd) vector comprising a polynucleotide encoding a hepatitis B surface antigen (HBs) and a nucleic add encoding a hepatitis B virus core antigen (HBc); and
c) a composition comprising a recombinant hepatitis B surface antigen (HBs), recombinant hepatitis B virus core antigen (HBc) and an adjuvant.

The immunogenic combination may find use in a method for treating chronic hepatitis B (CBH) and/or CHD by administration of the compositions in a prime-boost regimen.

The immunogenic combination may find use in a method for treating CHB and/or CHD in a human by administration of the compositions sequentially or concomitantly.

In one embodiment, the antisense oligonucleotide targeted to a HBV nucleic acid has the sequence GCAGAGGTGAAGCGAAGTGC. In one such embodiment, the antisense oligonucleotide targeted to a HBV nucleic acid is a modified oligonucleotide “gapmer” consisting of 20 linked nucleosides in which each internucleoside linkage is a phosphorothioate linkage and each cytosine is a 5-methylcytosine, having the sequence GCAGAGGTGAAGCGAAGTGC consisting of a 5′ wing segment consisting of five linked nucleosides GCAGA each comprising a 2′-O-methoxyethyl sugar, followed by ten linked deoxynucleosides GGTGAAGCGA and a 3′ wing segment consisting of five linked nucleosides AGTGC each comprising a 2′-O-methoxyethyl sugar.

### DESCRIPTION OF DRAWINGS/FIGURES

FIG. 1—HBc-(A) and HBs-(B) specific CD8⁺ T-cell responses at 7 days post-second and fourth dose of NaCl, heterologous vector prime-boost with subsequent recombinant proteins or heterologous vector prime-boost with concomitant recombinant proteins (individual animals with medians)

FIG. 2—HBc-(A) or HBs-(B) specific CD4⁺ T-cell responses at 7 days post-second and fourth dose of NaCl, heterologous vector prime-boost with subsequent recombinant proteins or heterologous vector prime-boost with concomitant recombinant proteins (individual animals with medians)

FIG. 3—HBc- and HBs-specific CD4⁺ (A) and CD8⁺ (B) T-cells in liver infiltrating lymphocytes 7 days post-fourth dose of NaCl, heterologous vector prime-boost with subsequent recombinant proteins or heterologous vector prime-boost with concomitant recombinant proteins (pools of 3 or 4 animals with medians)

FIG. 4—HBc-specific (A) and HBs-specific (B) antibody response after prime boost vaccine regimens (individual animals with geomeans are represented)

FIG. 5—HBc-specific spleen (A) or liver (B) CD8+ T cells at 7 days post-second dose and 7 days post-fourth dose of NaCl, heterologous vector prime-boost with subsequent recombinant proteins or heterologous vector prime-boost with concomitant recombinant proteins (individual animals with medians)

FIG. 6—HBc-specific spleen (A) or liver (B) CD4+ T cells at 7 days post-second dose and 7 days post-fourth dose of NaCl, heterologous vector prime-boost with subsequent recombinant proteins or heterologous vector prime-boost with concomitant recombinant proteins (individual animals with medians)

FIG. 7—HBs-specific spleen (A) or liver (B) CD4+ T cells at 7 days post-second dose and 7 days post-fourth dose of NaCl, heterologous vector prime-boost with subsequent recombinant proteins or heterologous vector prime-boost with concomitant recombinant proteins (individual animals with medians)

FIG. 8—HBs-specific spleen (A) or liver (B) CD4+ T cells at 7 days post-second dose and 7 days post-fourth dose of NaCl, heterologous vector prime-boost with subsequent recombinant proteins or heterologous vector prime-boost with concomitant recombinant proteins (individual animals with medians)

FIG. 9—Anti-HBs (A) and anti-HBc (B) binding antibody responses at Days 23, 65 and 93 (pre-dosing, 7 days post-second dose and 7 days post-fourth dose of NaCl, heterologous vector prime-boost with subsequent recombinant proteins or heterologous vector prime-boost with concomitant recombinant proteins)

FIG. 10—AST (A) and ALT (B) levels measured in sera from mice (groups 1, 2, 3 and 4) at Days 38, 65, and 93 (7 days post-first, second and post-fourth dose of NaCl, heterologous vector prime-boost with subsequent recombinant proteins or heterologous vector prime-boost with concomitant recombinant proteins groups 1, 2, 3) or at day 93 (group 4)

FIG. 11—HBs antigen levels in sera from AAV2/8-HBV injected mice pre-dosing, 7 days post-second dose and 7 days post-fourth dose of NaCl, heterologous vector prime-boost with subsequent recombinant proteins or heterologous vector prime-boost with concomitant recombinant proteins

FIG. 12—Structure of HBc-2A-HBs construct

FIG. 13—Structure of hIi-HBc-2A-HBs construct

### SEOUENCE LISTINGS

SEQ ID NO:1: Amino acid sequence of HBs
SEQ ID NO:2: Amino acid sequence of HBc truncate
SEQ ID NO:3: Amino acid sequence of spacer incorporating 2A cleavage region of foot and mouth virus
SEQ ID NO:4: Nucleotide sequence encoding spacer incorporating 2A cleavage region of foot and mouth virus
SEQ ID NO:5: Amino acid sequence of HBc-2A-HBs
SEQ ID NO:6: Nucleotide sequence encoding HBc-2A-HBs
SEQ ID NO:7: Amino acid sequence of hIi
SEQ ID NO:8: Nucleotide sequence encoding hIi
SEQ ID NO:9: Amino acid sequence of hIi-HBc-2A-HBs
SEQ ID NO:10: Nucleotide sequence encoding hIi-HBc-2A-HBs
SEQ ID NO:11: Amino acid sequence of HBc
SEQ ID NO:12: Amino acid sequence of hIi alternate variant
SEQ ID NO:13: Nucleotide sequence encoding hIi alternate variant
SEQ ID NO:14: Alternative nucleic acid sequence of hIi-HBc-2A-HBs
SEQ ID NO:15: Alternative amino acid sequence of hIi-HBc-2A-HBs
SEQ ID NO:16: Nucleotide sequence of Hepatitis B viral genome (GENBANK Accession No. U95551.1)

### DETAILED DESCRIPTION OF THE INVENTION

### Definitions

Unless defined otherwise, all technical and scientific terms used herein have the same meanings as commonly understood by one of ordinary skill in the art. For example, certain terms used herein are defined as described in “A multilingual glossary of biotechnological terms: (IUPAC Recommendations)”, Leuenberger, H. G. W, Nagel, B, and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. All definitions provided herein in the context of one aspect of the invention also apply to the other aspects of the invention.

“2′-O-methoxyethyl” (also 2′-MOE and 2′-O(CH₂)₂—OCH₃) refers to an O-methoxy-ethyl modification at the 2′ position of a furanose ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” means a nucleoside comprising a substituent at the 2′-position of the furanosyl ring other than H or OH. In certain embodiments, 2′ substituted nucleosides include nucleosides with bicyclic sugar modifications.

“5-methylcytosine” means a cytosine modified with a methyl group attached to the 5 position. A 5-methylcytosine is a modified nucleobase.

“About” means within ±7% of a value. For example, if it is stated, “the compounds affected about 70% inhibition of HBV”, it is implied that the HBV levels are inhibited within a range of 63% and 77%.

“Active pharmaceutical agent” means the substance or substances in a pharmaceutical composition that provide a therapeutic benefit when administered to an individual. For example, in certain embodiments an antisense oligonucleotide targeted to HBV is an active pharmaceutical agent.

“Acute hepatitis B infection” results when a person exposed to the hepatitis B virus begins to develop the signs and symptoms of viral hepatitis. The period of time between exposure and developing signs and symptoms of infection, called the incubation period, is an average of 90 days, but could be as short as 45 days or as long as 6 months. For most people this infection will cause mild to moderate discomfort but will go away by itself because of the body's immune response succeeds in fighting the virus. However, some people, particularly those with compromised immune systems, such as persons suffering from AIDS, undergoing chemotherapy, taking immunosuppressant drugs, or taking steroids, have very serious problems as a result of the acute HBV infection, and go on to more severe conditions such as fulminant liver failure.

“Chronic hepatitis B infection” occurs when a person initially suffers from an acute infection but is then unable to fight off the infection. About 90% of infants infected at birth will progress to chronic disease. However, as a person ages, the risk of chronic infection decreases such that between 20%-50% of people infected as children and less than 10% of older children or people infected as adults will progress from acute to chronic infection. Chronic HBV infections are the primary treatment goal for embodiments of the present invention, although compositions of the present invention are also capable of treating HBV-related conditions, such as inflammation, fibrosis, cirrhosis, liver cancer, serum hepatitis etc.

“Peptide” means a molecule formed by linking at least two amino acids by amide bonds (also referred to as peptide bonds). The terms “protein”, “polypeptide” and “peptide” are used interchangeably herein and refer to any peptide-linked chain of amino acids, regardless of length, co-translational or post-translational modification. A “fusion protein” (or “chimeric protein”) is a recombinant protein comprising two or more peptide-linked proteins. Fusion proteins are created through the joining of two or more genes that originally coded for the separate proteins. Translation of this fusion gene results in a single fusion protein. In relation to a protein or polypeptide, recombinant means that the protein is expressed from a recombinant polynucleotide.

The terms “polynucleotide” and “nucleic acid” are used interchangeably herein and refer to a polymeric macromolecule made from nucleotide monomers. Suitably the polynucleotides of the invention are recombinant. Recombinant means that the polynucleotide is the product of at least one of cloning, restriction or ligation steps, or other procedures that result in a polynucleotide that is distinct from a polynucleotide found in nature.

A heterologous nucleic acid sequence refers to any nucleic acid sequence that is not isolated from, derived from, or based upon a naturally occurring nucleic acid sequence found in the host organism. “Naturally occurring” means a sequence found in nature and not synthetically prepared or modified. A sequence is “derived” from a source when it is isolated from a source but modified (e.g., by deletion, substitution (mutation), insertion, or other modification), suitably so as not to disrupt the normal function of the source gene.

Suitably, the polynucleotides used in the present invention are isolated. An “isolated” polynucleotide is one that is removed from its original environment. For example, a naturally-occurring polynucleotide is isolated if it is separated from some or all of the coexisting materials in the natural system. A polynucleotide is considered to be isolated if, for example, it is cloned into a vector that is not a part of its natural environment or if it is comprised within cDNA.

“Treatment” refers to administering a composition to affect an alteration or improvement of the disease or condition. The term “treating” as used herein in relation to chronic hepatitis B infection refers to the administration of suitable compositions with the intention of reducing the symptoms of CHB, preventing the progression of CHB or reducing the level of one or more detectable markers of CHB. For example, preventing the progression of CHB may include preventing the onset of liver disease or stabilising pre-existing liver disease, as indicated by ALT (alanine transaminase) levels, liver fibrosis or other suitable detectable markers. Other markers of CHB include the serum HBV DNA level, which is an indicator of viral replication and the serum HBs antigen level, which is an indicator of viral load, thus treating CHB may include reducing the level of serum HBsAg (e.g. as determined by quantitative immunoassay) or HBV DNA (e.g. as determined by the Cobas® HBV assay (Roche) or equivalent) to undetectable levels (“clearing” HBsAg or HBV DNA). The term “treating” as used herein in relation to chronic hepatitis D infection (CHD) is to be interpreted accordingly.

“Administering” means providing a pharmaceutical agent to an individual, and includes, but is not limited to administering by a medical professional and self-administering.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. “Concomitant” administration as used herein in relation to the components of a vaccine regimen refers to administration during the same ongoing immune response and “concomitantly” is to be interpreted accordingly. Preferably both components are administered at the same time (such as concomitant administration of a composition comprising a vector and a composition comprising a protein), however, one component could be administered within a few minutes (for example, at the same medical appointment or doctor's visit), or within a few hours of the other component. Such administration is also referred to as co-administration. Concomitant administration of separate components may occur via the same route of administration e.g. intramuscular injection. Alternatively, concomitant administration of separate components may occur via different routes of administration e.g. intramuscular injection and intradermal injection, intramuscular and intranasal administration, inhalation and subcutaneous administration etc. In some embodiments, concomitant administration may refer to the administration of an adenoviral vector, and a protein component. In other embodiments, co-administration refers to the administration of an adenoviral vector and another viral vector, for example a poxvirus such as MVA. In other embodiments, co-administration refers to the administration of an adenoviral vector and a protein component, in which the protein component is adjuvanted.

“Sequential” administration refers to administration of a first composition, followed by administration of a second composition a significant time later. The period of time between two sequential administrations is between 1 week and 12 months, for example between 2 weeks and 12 weeks, for example, 1 week, 2 weeks, 4 weeks, 6 weeks 8 weeks or 12 weeks, 6 months or 12 months. More particularly, it is between 4 weeks and 8 weeks, for example the period of time between sequential administrations may be 4 weeks. Thus, sequential administration encompasses a first and a subsequent administration in a prime-boost setting, i.e. when the administration of the second composition is not carried out during the ongoing immune response engendered by the first administration.

“Immunogenic combination” as used herein refers to a plurality of separately formulated immunogenic compositions administered sequentially and/or concomitantly in a single immunisation regimen, e.g. a prime-boost regimen, each separately formulated immunogenic composition being a component of the immunogenic combination.

“Antisense compound” means an oligomeric compound that is is capable of undergoing hybridization to a target nucleic acid through hydrogen bonding. Examples of antisense compounds include single-stranded and double-stranded compounds, such as, antisense oligonucleotides, siRNAs, shRNAs, snoRNAs, miRNAs, and satellite repeats.

“Antisense inhibition” means reduction of target nucleic acid levels in the presence of an antisense compound complementary to a target nucleic acid compared to target nucleic acid levels in the absence of the antisense compound.

“Antisense oligonucleotide” means a single-stranded oligonucleotide having a nucleobase sequence that permits hybridization to a corresponding region or segment of a target nucleic acid.

“Complementarity” means the capacity for pairing between nucleobases of a first nucleic acid and a second nucleic acid

“Base complementarity” refers to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Hybridization” means the annealing of complementary nucleic acid molecules. In certain embodiments, complementary nucleic acid molecules include, but are not limited to, an antisense compound and a nucleic acid target. In certain embodiments, complementary nucleic acid molecules include, but are not limited to, an antisense oligonucleotide and a nucleic acid target.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Contiguous nucleobases” means nucleobases immediately adjacent to each other.

“Contiguous nucleobases” means nucleobases immediately adjacent to each other.

“Deoxyribonucleotide” means a nucleotide having a hydrogen at the 2′ position of the sugar portion of the nucleotide. Deoxyribonucleotides may be modified with any of a variety of substituents.

“Diluent” means an ingredient in a composition that lacks pharmacological activity, but is pharmaceutically necessary or desirable. For example, in drugs that are injected, the diluent may be a liquid, e.g. saline solution.

“Dosage unit” means a form in which a pharmaceutical agent is provided, e.g. pill, tablet, or other dosage unit known in the art.

“Dose” means a specified quantity of a pharmaceutical agent provided in a single administration, or in a specified time period. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual. In other embodiments, the pharmaceutical agent is administered by infusion over an extended period of time or continuously. Doses may be stated as the amount of pharmaceutical agent per hour, day, week or month.

“Dosing regimen” is a combination of doses designed to achieve one or more desired effects.

“HBV” means mammalian hepatitis B virus, including human hepatitis B virus. The term encompasses geographical genotypes of hepatitis B virus, particularly human hepatitis B virus, as well as variant strains of geographical genotypes of hepatitis B virus.

“HBV antigen” means any hepatitis B virus antigen or protein, including core proteins such as “hepatitis B core antigen” or “HBcAg” and “hepatitis B E antigen” or “HBeAG” and envelope proteins such as “HBV surface antigen”, or “HBsAg”.

“Hepatitis B E antigen” or “HBeAg” is a secreted, non-particulate form of HBV core protein. HBV antigens HBeAg and HBcAg share primary amino acid sequences, so show cross-reactivity at the T cell level. HBeAg is not required for viral assembly or replication, although studies suggest they may be required for establishment of chronic infection.

“HBV surface antigen”, or “HBsAg”, or “HBsAG” is the envelope protein of infectious HBV viral particles but is also secreted as a non-infectious particle (Dane particle) with serum levels 1000-fold higher than HBV viral particles. The serum levels of HBsAg in an infected person or animal can be as high as 1000 μg/mL (Kann and Gehrlich (1998) Topley & Wilson's Microbiology and Microbial Infections, 9^{th} ed. 745).

“Hepatitis B-related condition” or “HBV-related condition” means any disease, biological condition, medical condition, or event which is exacerbated, caused by, related to, associated with, or traceable to a hepatitis B infection, exposure, or illness. The term hepatitis B-related condition includes chronic HBV infection, inflammation, fibrosis, cirrhosis, liver cancer, serum hepatitis, jaundice, liver cancer, liver inflammation, liver fibrosis, liver cirrhosis, liver failure, diffuse hepatocellular inflammatory disease, hemophagocytic syndrome, serum hepatitis, HBV viremia, liver disease related to transplantation, and conditions having symptoms which may include any or all of the following: flu-like illness, weakness, aches, headache, fever, loss of appetite, diarrhoea, nausea and vomiting, pain over the liver area of the body, clay- or grey-colored stool, itching all over, and dark-colored urine, when coupled with a positive test for presence of a hepatitis B virus, a hepatitis B viral antigen, or a positive test for the presence of an antibody specific for a hepatitis B viral antigen.

“Inhibiting the expression or activity” refers to a reduction, blockade of the expression or activity and does not necessarily indicate a total elimination of expression or activity.

“Internucleoside linkage” refers to the chemical bond between nucleosides.

“Linked nucleosides” means adjacent nucleosides linked together by an internucleoside linkage.

“Modified internucleoside linkage” refers to a substitution or any change from a naturally occurring internucleoside bond (i.e. a phosphodiester internucleoside bond).

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C) and uracil (U).

“Modified nucleoside” means a nucleoside having, independently, a modified sugar moiety and/or modified nucleobase.

“Modified nucleotide” means a nucleotide having, independently, a modified sugar moiety, modified internucleoside linkage, or modified nucleobase.

“Modified oligonucleotide” means an oligonucleotide comprising at least one modified internucleoside linkage, a modified sugar, and/or a modified nucleobase.

“Modified sugar” means substitution and/or any chance from a natural sugar moiety.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Motif” means the pattern of unmodified and modified nucleosides in an antisense compound.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Wing segment” means a plurality of nucleosides modified to impart to an oligonucleotide properties such as enhanced inhibitory activity, increased binding affinity for a target nucleic acid, or resistance to degradation by in vivo nucleases.

“Natural sugar moiety” means a sugar moiety found in DNA (2′-H) or RNA (2′-OH).

“Unmodified” nucleobases mean the purine bases adenine (A) and guanine (G), and the pyrimidine bases thymine (T), cytosine (C) and uracil (U).

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” means the order of contiguous nucleobases independent of any sugar, linkage, and/or nucleobase modification.

“Nucleoside” means a nucleobase linked to a sugar.

“Nucleotide” means a nucleoside having a phosphate group covalently linked to the sugar portion of the nucleoside.

“Oligonucleotide” means a polymer of linked nucleosides each of which can be modified or unmodified, independent one from another.

“Parenteral administration” means administration through injection (e.g., bolus injection) or infusion. Parenteral administration includes subcutaneous administration, intravenous administration, intramuscular administration, intraarterial administration, intraperitoneal administration, or intracranial administration, e.g., intrathecal or intracerebroventricular administration.

“Pharmaceutical composition” means a mixture of substances suitable for administering to a subject. For example, a pharmaceutical composition suitable for administration by injection may comprise an antisense oligonucleotide and/or a vaccine component and a sterile aqueous solution.

“Subject” means a human or non-human animal selected for treatment or therapy.

With regard to percentage homologies, looking at a pairwise alignment of two sequences, aligned identical residues (‘identities’) between the two sequences can be observed, A percentage of identity (or homology), can be calculated by multiplying by 100 (a) the quotient between the number of identities and the full length of the reference sequence (i.e. Percentage identity=(Number of identities×100)/Length of reference sequence.

### Regimens

The present disclosure encompasses a regimen which provides for a schedule of antisense oligonucleotide (ASO) treatment followed by a heterologous prime-boost vaccine schedule involving at least one viral vector coding for the hepatitis B core (HBc) and the hepatitis B surface (HBs) antigens, in order to induce a strong CD8⁺ T-cell response, with sequential or concomitant administration of adjuvanted recombinant HBc and HBs proteins in order to induce strong antigen-specific CD4⁺ T-cell and antibody responses. The disclosed ASO treatment successfully inhibits target HBV DNA and RNA in liver cells in vivo and in vitro. The disclosed vaccine regimens successfully restore HBs- and HBc-specific antibody and CD8⁺ T cell responses as well as HBs-specific CD4⁺ T cell responses, without associated signs of liver alteration side effects, in a mouse model which recapitulates virological and immunological characteristics of human chronic HBV infection. Together, the combined ASO and vaccine regimen will provide for a virological and clinical response, including loss of HBsAg and/or HBsAg seroconversion, with induction of a robust poly-functional CD8⁺ T-cell response to HBV core antigen (HBcAg).
More specifically, there is provided a method of treating chronic hepatitis B infection (CHB) and/or chronic hepatitis D infection (CHD) in a human, comprising the steps of:
a) administering to the human a composition comprising an antisense oligonucleotide 10 to 30 nucleosides in length, targeted to a HBV nucleic acid (an HBV ASO);
b) administering to the human a composition comprising a replication-defective chimpanzee adenoviral (ChAd) vector comprising a polynucleotide encoding a hepatitis B surface antigen (HBs) and a nucleic add encoding a hepatitis B virus core antigen (HBc);
c) administering to the human a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) administering to the human a composition comprising a recombinant hepatitis B surface antigen (HBs), recombinant hepatitis B virus core antigen (HBc) and an adjuvant.

In one embodiment, the steps of the method are carried out sequentially, with step a) preceding step b), step b) preceding step c) and step c) preceding step d). Optionally step a) may be repeated. Optionally, step c) may be repeated. In certain embodiments the period of time between the steps of the method is 2 to 12 weeks, for example 2 weeks, 3 weeks, 4 weeks, 5 weeks, 6 weeks, 7 weeks, 8 weeks, 9 weeks, 10 weeks, 11 weeks or 12 weeks. In one embodiment the period of time between the steps of the method is 4 to 8 weeks. In one embodiment, the period of time between sequential administrations of compositions according to the method is 4 weeks. In one embodiment, step a) is carried out from 2 to 12 times at weekly intervals or two-weekly intervals, or every 3 weeks or every 4 weeks, for example from 2 to 10 times, from 2 to 8 times, from 2 to 7 times, from 2 to 6 times, from 2 to 5 times, for example 4 times, 3 times or twice. In a particular embodiment, step a) is carried out from 2 to 10 times at weekly intervals, from 2 to 8 times at weekly intervals, from 2 to 7 times at weekly intervals, from 2 to 6 times at weekly intervals, from 2 to 5 times at weekly intervals, for example 4 times at weekly intervals, 3 times at weekly intervals or twice, a week apart. In another embodiment, step a) is repeated daily then repeated weekly. For example step a) may be carried out daily from 2 to 4 times, then carried out from 2 to 8 times at weekly intervals. In another embodiment, step a) is repeated three times, on day 1, day 3 and day 5 of the regimen, then from 2 to 8 times, from 2 to 6 times, from 2 to 4 times e.g. 4 times, 3 times or twice at weekly intervals commencing on day 12 of the regimen. In a further embodiment, step a) is carried out from 4 to 8 times over a period of 20-36 days, for example on days 1, 4, 8, 11, 15, 22, 26 and day 30 of the regimen, or on days 1, 4, 8, 11, 15 and 22 of the regimen, or on days 1, 6, 11, 16, 21, 26, 31 and day 36 of the regimen. In one embodiment, step a) is carried out daily, on alternate days and/or at weekly intervals prior to step b), step b) is carried out prior to step c) and step c is carried out prior to step d). In another embodiment, step a) is carried out daily, on alternate days and/or at weekly intervals prior to step b) and is repeated at weekly intervals during the time period over which step b), step c) and/or step d) are carried out. In another embodiment, step d) is carried out concomitantly with step a) and/or with step b) and/or with step c). In certain embodiments, concomitant steps b) and c) may be repeated. In certain embodiments, concomitant steps c) and d) may be repeated. In one embodiment, the steps of the method are carried out sequentially, with step a), optionally repeated, preceding step c), step c) preceding step b) and step d) either following step b), or carried out concomitantly with step b) and/or with step c). In one embodiment, the steps of the method are carried out sequentially, with step a), optionally repeated, preceding step d), step d) preceding step b) and step b) preceding step c). In another embodiment, the steps of the method are carried out sequentially, with step a), optionally repeated, preceding step d), step d) preceding step c) and step c) preceding step b). In a further embodiment, step d is repeated and the steps of the method are carried out in the following order: step a) (optionally repeated), step b), step c), step d), step d). In certain embodiments the period of time between the steps b), c) and d) of the method is 2 to 12 weeks, for example 2 weeks, 3 weeks, 4 weeks, 5 weeks, 6 weeks, 7 weeks, 8 weeks, 9 weeks, 10 weeks, 11 weeks or 12 weeks. In one embodiment the period of time between the steps b), c) and d) of the method is 4 to 8 weeks. In one embodiment, the period of time between sequential administrations of compositions according to steps b), c) and d) of the method is 4 weeks. In certain embodiments the method is carried out over a period of one year. In certain embodiments, the method is carried out over a period of 8 to 50 weeks, for example 8 to 40 weeks, 8 to 30 weeks, 8 to 20 weeks, 8 to 16 weeks, for example the method may be carried out over 8 weeks, 9 weeks, 10 weeks, 12 weeks, 14 weeks, 16 weeks, over a period of 10 to 16 weeks, 12 to 16 weeks, 16 to 20 weeks, 20 to 40 weeks or 30 to 50 weeks.

In another aspect, there is provided a method of treating chronic hepatitis B infection (CHB) and/or chronic hepatitis D infection (CHD) in a human, comprising the steps of:
a) administering to the human a composition comprising an antisense oligonucleotide (ASO) 10 to 30 nucleosides in length, targeted to a HBV nucleic acid (an HBV ASO);
b) administering to the human a composition comprising a replication-defective chimpanzee adenoviral (ChAd) vector comprising a polynucleotide encoding a hepatitis B surface antigen (HBs) and a nucleic acid encoding a hepatitis B virus core antigen (HBc); and
c) administering to the human a composition comprising a recombinant hepatitis B surface antigen (HBs), a recombinant hepatitis B virus core antigen (HBc) and an adjuvant.

In one embodiment, the steps of the method are carried out sequentially, with step a) preceding step b) and step b) preceding step c). Optionally step a) may be repeated. Optionally, step c) may be repeated. In another embodiment, step c) is carried out concomitantly with step b). In certain embodiments the period of time between the steps b) and c) of the method is 2 to 12 weeks, for example 2 weeks, 3 weeks, 4 weeks, 5 weeks, 6 weeks, 7 weeks, 8 weeks, 9 weeks, 10 weeks, 11 weeks or 12 weeks. In one embodiment the period of time between the steps b) and c) of the method is 4 to 8 weeks. In one embodiment, step a) is carried out from 2 to 12 times at weekly intervals or two-weekly intervals, or every 3 weeks or every 4 weeks, for example from 2 to 10 times, from 2 to 8 times, from 2 to 7 times, from 2 to 6 times, from 2 to 5 times, for example 4 times, 3 times or twice. In a particular embodiment, step a) is carried out from 2 to 10 times at weekly intervals, from 2 to 8 times at weekly intervals, from 2 to 7 times at weekly intervals, from 2 to 6 times at weekly intervals, from 2 to 5 times at weekly intervals, for example 4 times at weekly intervals, 3 times at weekly intervals or twice, a week apart. In another embodiment, step a) is repeated daily then repeated weekly. For example step a) may be carried out daily from 2 to 4 times, then carried out from 2 to 8 times at weekly intervals. In another embodiment, step a) is repeated three times, on day 1, day 3 and day 5 of the regimen, then from 2 to 8 times, from 2 to 6 times, from 2 to 4 times e.g. 4 times, 3 times or twice at weekly intervals commencing on day 12 of the regimen. In a further embodiment, step a) is carried out from 4 to 8 times over a period of 20-36 days, for example on days 1, 4, 8, 11, 15, 22, 26 and day 30 of the regimen, or on days 1, 4, 8, 11, 15 and 22 of the regimen, or on days 1, 6, 11, 16, 21, 26, 31 and day 36 of the regimen. In one embodiment, step a) is carried out daily, on alternate days and/or at weekly intervals prior to step b) and step b) is carried out prior to step c). In another embodiment, step a) is carried out daily, on alternate days and/or at weekly intervals prior to step b) and is repeated at weekly intervals during the time period over which step b) and step c) are carried out. In another embodiment, step c) is carried out concomitantly with step a) and/or with step b). In certain embodiments, concomitant steps b) and c) may be repeated. In one embodiment, the steps of the method are carried out sequentially, with step a), optionally repeated, preceding step c) and step c) preceding step b). In certain embodiments the method is carried out over a period of one year. In certain embodiments, the method is carried out over a period of 8 to 50 weeks, for example 8 to 40 weeks, 8 to 30 weeks, 8 to 20 weeks, 8 to 16 weeks, for example the method may be carried out over 8 weeks, 9 weeks, 10 weeks, 12 weeks, 14 weeks, 16 weeks, over a period of 10 to 16 weeks, 12 to 16 weeks, 16 to 20 weeks, 20 to 40 weeks or 30 to 50 weeks.

In certain embodiments, the composition administered in step a) of the method comprises an oligonucleotide 10 to 30 linked nucleosides in length targeted to a HBV nucleic acid (an HBV ASO). The HBV target has a sequence comprised within the sequence of SEQ ID NO: 16. Thus, in certain embodiments the HBV ASO targets a region of a HBV nucleic acid. In certain embodiments, the composition administered in step a) comprises an HBV ASO having a contiguous nucleobase portion that is complementary to an equal length nucleobase portion of the targeted region of the HBV nucleic acid of SEQ ID NO: 16. For example, the contiguous nucleobase portion of the HBV ASO can be at least an 8, 9, 10, 11, 12, 13, 14, 15, 16, 17, 18, 19 or 20 contiguous nucleobases complementary to an equal length portion of a region SEQ ID NO: 16. In certain embodiments, the composition administered in step a) comprises an antisense oligonucleotide targeted to a HBV nucleic acid is complementary within one of the following nucleotide regions of SEQ ID NO: 16: 58-73, 58-74, 58-77, 59-74, 59-75, 60-75, 60-76, 61-76, 61-77, 62-77, 253-272, 253-269, 254-270, 255-271, 256-272, 411-437, 411-426, 411-427, 411-430, 412-427, 412-428, 412-431, 413-428, 413-429, 413-432, 414-429, 414-430, 414-433, 415-430, 415-431, 415-434, 416-431, 416-432, 416-435, 417-432, 417-433, 417-436, 418-433, 418-434, 418-437, 457-472, 457-473, 458-473, 670-706, 670-685, 670-686, 671-686, 671-687, 672-687, 672-688, 673-688, 687-702, 687-703, 687-706, 688-703, 688-704, 689-704, 689-705, 690-705, 690-706, 691-706, 1261-1285, 1261-1276, 1261-1277, 1261-1280, 1262-1277, 1262-1278, 1262-1281, 1263-1278, 1263-1279, 1263-1282, 1264-1279, 1264-1280, 1264-1283, 1265-1280, 1265-1281, 1265-1284, 1266-1281, 1266-1282, 1266-1285, 1267-1282, 1267-1283, 1268-1283, 1268-1284, 1269-1284, 1269-1285, 1270-1285, 1577-1606, 1577-1592, 1577-1593, 1577-1596, 1578-1593, 1578-1594, 1578-1597, 1579-1594, 1579-1594, 1579-1598, 1580-1595, 1580-1596, 1580-1599, 1581-1596, 1581-1597, 1581-1600, 1582-1597, 1582-1598, 1582-1601, 1583-1598, 1583-1599, 1583-1602, 1584-1599, 1584-1600, 1584-1603, 1585-1600, 1585-1601, 1585-1604, 1586-1601, 1586-1602, 1586-1605, 1587-1602, 1587-1603, 1587-1606, 1588-1603, 1588-1604, 1589-1604, 1589-1605, 1590-1605, 1590-1606, 1591-1606, 1778-1800, 1778-1793, 1778-1794, 1778-1797, 1779-1794, 1779-1795, 1779-1798, 1780-1795, 1780-1796, 1780-1799, 1781-1796, 1781-1797, 1781-1800, 1782-1797, 1782-1798, 1783-1798, 1783-1799, 1784-1799, and 1784-1800.

In certain embodiments, the composition administered in step a) comprises an HBV ASO in which the contiguous nucleobase portion is 16, 17, 18, 19 or 20 contiguous nucleobases complementary to an equal length portion of a region a HBV nucleic acid of SEQ ID NO: 16. In a particular embodiment an antisense oligonucleotide targeted to a HBV nucleic acid has 16-20 complementary contiguous nucleobases complementary to one of the following nucleotide regions of SEQ ID NO: 16: 58-77, 253-272, 411-430, 412-431, 413-432, 414-433, 415-434, 416-435, 417-436, 418-437, 687-706, 1261-1280, 1262-1281, 1263-1282, 1264-1283, 1265-1284, 1266-1285, 1577-1596, 1578-1597, 1579-1598, 1580-1599, 1581-1600, 1582-1601, 15834602, 1584-1603, 1585-1604, 1586-1605, 1587-1606, 1778-1797, 1779-1798, 1780-1799 and 1781-1800 or a portion thereof. In a particular embodiment an antisense oligonucleotide targeted to a HBV nucleic acid has 20 complementary contiguous nucleobases complementary to one of the following nucleotide regions of SEQ ID NO: 16: 58-77, 253-272, 411-430, 412-431, 413-432, 414-433, 415-434, 416-435, 417-436, 418-437, 687-706, 1261-1280, 1262-1281, 1263-1282, 1264-1283, 1265-1284, 1266-1285, 1577-1596, 1578-1597, 1579-1598, 1580-1599, 1581-1600, 1582-1601, 1583-1602, 1584-1603, 1585-1604, 1586-1605, 1587-1606, 1778-1797, 1779-1798, 1780-1799 and 1781-1800.

In certain embodiments, the composition administered in step a) comprises an antisense oligonucleotide targeted to a HBV nucleic acid complementary within the following nucleotide region of SEQ ID NO: 16: 1583-1602. In a particular embodiment, an antisense oligonucleotide targeted to a HBV nucleic acid has 16-20 complementary contiguous nucleobases complementary within the following nucleotide region of SEQ ID NO: 16: 1583-1602. In a particular embodiment, an antisense oligonucleotide targeted to a HBV nucleic acid has 20 complementary contiguous nucleobases complementary to the following nucleotide region of SEQ ID NO: 16: 1583-1602.

In certain embodiments, the composition administered in step a) comprises an antisense oligonucleotide having a nucleotide sequence selected from SEQ ID NOs: 83-310 of WO2012/145697 (PCT/US2012/034550, filed Apr. 20, 2012). In particular embodiments, the antisense oligonucleotide targeted to a HBV nucleic acid (HBV ASO) has a nucleotide sequence selected from SEQ ID NOs: 224-227 of WO2012/145697, or a sequence having 85-95% identity to a sequence selected from SEQ ID NOs: 224-227 of WO2012/145697. In a particular embodiment, the HBV ASO administered in step a) of the method has 85-95% identity to the sequence GCAGAGGTGAAGCGAAGTGC (SEQ ID NO: 226 of WO2012/145697) complementary to the HBV genome sequence SEQ ID NO: 16 at nucleobases 1583-1602. In a particular embodiment, the HBV ASO administered in step a) of the method has the sequence GCAGAGGTGAAGCGAAGTGC (SEQ ID NO: 226 of WO2012/145697) complementary to the HBV genome sequence SEQ ID NO: 16 at nucleobases 1583-1602.

In certain embodiments, the composition administered in step b) of the method comprises a ChAd vector selected from the group consisting of ChAd3, ChAd63, ChAd83, ChAd155, ChAd157, Pan 5, Pan 6, Pan 7 (also referred to as C7) and Pan 9, in particular, ChAd63 or ChAd155. In certain embodiments the ChAd vector includes a vector insert encoding HBc and HBs, separated by a sequence encoding the 2A cleaving region of the foot and mouth disease virus. In certain embodiments, the vector insert encodes HBc (e.g. SEQ ID NO:11 or an amino acid sequence at least 98% homologous thereto) and HBs (e.g. SEQ ID NO:1 or an amino acid sequence at least 98% homologous thereto), separated by a sequence encoding a spacer which incorporates the 2A cleaving region of the foot and mouth disease virus (e.g. SEQ ID NO:3 or an amino acid sequence at least 98% homologous thereto). In certain embodiments, HBc (e.g. SEQ ID NO:11 or an amino acid sequence at least 98% homologous thereto) is fused to hIi (e.g. SEQ ID NO:7 or an amino acid sequence at least 98% homologous thereto or SEQ ID NO:12, or an amino acid sequence at least 98% homologous thereto). For example, HBc (e.g. SEQ ID NO:11) is fused to hIi (e.g. SEQ ID NO:7), or HBc (e.g. SEQ ID NO:11) is fused to hIi (e.g. SEQ ID NO:12). In a particular embodiment, the composition administered in step b) of the method comprises a ChAd155 vector which comprises a polynucleotide vector insert encoding hIi, HBc, 2A and HBs, for example, an insert encoding a construct having the structure shown in FIG. 13. In one embodiment, the composition administered in step b) of the method comprises a ChAd vector which comprises a polynucleotide vector insert encoding the amino acid sequence of SEQ ID NO:9 or the amino acid sequence of SEQ ID NO:15. In certain embodiments, the composition administered in step b) of the method comprises a ChAd vector which comprises a polynucleotide vector insert having the nucleotide sequence given in SEQ ID NO:10 or the nucleotide sequence given in SEQ ID NO:14. In one specific embodiment, the vector is a ChAd155 vector. Thus, in certain embodiments, the composition administered in step b) of the method comprises a ChAd155 vector which comprises a polynucleotide vector insert encoding the amino acid sequence of SEQ ID NO:9. In other embodiments, the composition administered in step b) of the method comprises a ChAd155 vector which comprises a polynucleotide vector insert encoding the amino acid sequence of SEQ ID NO:15. In one embodiment, the composition administered in step b) of the method comprises a ChAd155 vector which comprises a polynucleotide vector insert having the nucleotide sequence given in SEQ ID NO:10. In other embodiments, the composition administered in step b) of the method comprises a ChAd155 vector which comprises a polynucleotide vector insert having the nucleotide sequence given in SEQ ID NO:14.

In one embodiment, the composition administered in step c) of the method comprises an MVA vector which includes a vector insert encoding HBc and HBs, separated by a sequence encoding the 2A cleaving region of the foot and mouth disease virus. In certain embodiments, the vector insert encodes HBc and HBs, separated by a sequence encoding a spacer which incorporates the 2A cleaving region of the foot and mouth disease virus. In a particular embodiment, the composition administered in step c) of the method comprises an MVA vector which comprises a polynucleotide vector insert encoding HBc, 2A and HBs, for example, an insert encoding a construct having the structure shown in FIG. 12. In certain embodiments, the vector insert encodes HBc (e.g. SEQ ID NO:11 or an amino acid sequence at least 98% homologous thereto) and HBs (e.g. SEQ ID NO:1 or an amino acid sequence at least 98% homologous thereto), separated by a sequence encoding a spacer which incorporates the 2A cleaving region of the foot and mouth disease virus (e.g. SEQ ID NO:3 or an amino acid sequence at least 98% homologous thereto). In one embodiment, the composition administered in step c) of the method comprises an MVA vector which comprises a polynucleotide vector insert encoding the amino acid sequence of SEQ ID NO:5. In one embodiment, the composition administered in step c) of the method comprises an MVA vector which comprises a polynucleotide vector insert having the nucleotide sequence given in SEQ ID NO:6.

In one embodiment, the composition administered in step d) of the method comprises recombinant HBc and recombinant HBs in a 1:1 ratio. In another embodiment the ratio of HBc to HBs in the composition is greater than 1, for example the ratio of HBc to HBs may be 1.5:1, 2:1, 2.5:1, 3:1, 3.5:1, 4:1, 4.5:1, 5:1, 5.5:1, 6:1 or more, especially 3:1 to 5:1, such as 3:1, 4:1 or 5:1, particularly a ratio of 4:1. In particular embodiments, the composition administered in step d) of the method comprises recombinant HBc and recombinant HBs in a ratio of 4:1 or more. In certain embodiments, the composition administered in step d) of the method comprises a full length recombinant hepatitis B surface antigen (HBs) (e.g. SEQ ID NO:1 or an amino acid sequence at least 98% homologous thereto), a recombinant hepatitis B virus core antigen (HBc) truncated at the C-terminal, and an adjuvant. In certain embodiments, the truncated recombinant HBc comprises the assembly domain of HBc, for example amino acids 1-149 of HBc (e.g. SEQ ID NO:2 or an amino acid sequence at least 98% homologous thereto). In one embodiment, the composition administered in step d) of the method comprises a full length recombinant HBs, amino acids 1-149 of HBc and an adjuvant comprising MPL and QS-21. For example, the composition administered in step d) of the method comprises a full length recombinant HBs (SEQ ID NO: 1), amino acids 1-149 of HBc (SEQ ID NO: 2) and an adjuvant comprising MPL and QS-21. In certain embodiments the recombinant protein HBs and HBc antigens are in the form of virus-like particles.

In a further embodiment, there is provided a method of treating CHB and/or chronic hepatitis D infection (CHD) in a human, comprising the steps of:
a) administering to the human a composition comprising an antisense oligonucleotide 10 to 30 nucleosides in length, targeted to a HBV nucleic acid (an HBV ASO);
b) administering to the human a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) administering to the human a composition comprising a Modified Vaccinia Virus Ankara (MVA) vector comprising a polynucleotide encoding a hepatitis B surface antigen (HBs) and a nucleic acid encoding a hepatitis B virus core antigen (HBc);
d) administering to the human a composition comprising a recombinant hepatitis B surface antigen (HBs), a recombinant hepatitis B virus core antigen (HBc) and an adjuvant; and
e) administering to the human a composition comprising a recombinant hepatitis B surface antigen (HBs), a recombinant hepatitis B virus core antigen (HBc) and an adjuvant.

In a particular embodiment, the HBV ASO administered in step a) of the method has 85-95% identity to the sequence GCAGAGGTGAAGCGAAGTGC (SEQ ID NO: 226 of WO2012/145697) complementary to the HBV genome sequence SEQ ID NO: 16 at nucleobases 1583-1602. In a particular embodiment, the HBV ASO administered in step a) of the method has the sequence GCAGAGGTGAAGCGAAGTGC (SEQ ID NO: 226 of WO2012/145697) complementary to the HBV genome sequence SEQ ID NO: 16 at nucleobases 1583-1602.

In another aspect of the present invention, there is provided a method of treating chronic hepatitis B infection (CHB) and/or chronic hepatitis D infection (CHD) in a human, comprising the steps of:
a) administering to the human a composition comprising an antisense oligonucleotide 1d to 30 nucleosides in length, targeted to a HBV nucleic acid (an HBV ASO);
b) administering to the human i) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc) and, concomitantly, ii) a composition comprising a recombinant hepatitis B surface antigen (HBs), a recombinant hepatitis B virus core antigen (HBc) and an adjuvant; and
c) administering to the human i) a composition comprising a Modified Vaccinia Virus Ankara (MVA) vector comprising a polynucleotide encoding a hepatitis B surface antigen (HBs) and a nucleic acid encoding a hepatitis B virus core antigen (HBc) and, concomitantly, a composition comprising a recombinant hepatitis B surface antigen (HBs), a recombinant hepatitis B virus core antigen (HBc) and an adjuvant.

In a particular embodiment, the HBV ASO administered in step a) of the method has 85-95% identity to the sequence GCAGAGGTGAAGCGAAGTGC (SEQ ID NO: 226 of WO2012/145697) complementary to the HBV genome sequence SEQ ID NO: 16 at nucleobases 1583-4602. In a particular embodiment, the HBV ASO administered in step a) of the method has the sequence GCAGAGGTGAAGCGAAGTGC (SEQ ID NO: 226 of WO2012/145697) complementary to the HBV genome sequence SEQ ID NO: 16 at nucleobases 1583-1602.

In one embodiment of this aspect of the invention, the steps of the method are carried out sequentially, with step a) preceding step b) and step b) preceding step c). Optionally, step a) may be repeated. Optionally step b) may be repeated. Optionally, step c) may be repeated. In one embodiment, the method steps are carried out in the order: step a) followed by step a) followed by step b) followed by step c). In an alternative embodiment, the method steps are carried out in the order: step a) followed by step b) followed by step c) followed by step c). In one embodiment, the method steps are carried out in the order: step a) followed by step b) followed by step b) followed by step c). Optionally, step a) may be repeated more than once. Optionally both step a) and step c) may be repeated. In one embodiment of this aspect of the invention, the method steps are carried out in the order: step a) followed by step a) followed by step b) followed by step c) followed by step c). In an alternative embodiment, the method steps are carried out in the order: step b) followed by step a) followed by step b) followed by step b). In a further embodiment, the method steps are carried out in the order: step a) repeated from 2 to 8 times followed by step b) followed by step c), followed by step c), optionally followed by step c). In certain embodiments the period of time between the steps of the method is 2 to 12 weeks, for example 2 weeks, 3 weeks, 4 weeks, 5 weeks, 6 weeks, 7 weeks, 8 weeks, 9 weeks, 10 weeks, 11 weeks or 12 weeks. In one embodiment the period of time between the steps of the method is 4 to 8 weeks. In one embodiment, the period of time between sequential administrations of compositions according to the method is 4 weeks.

Thus, in another embodiment of this aspect of the invention, there is provided a method of treating CHB and/or CHD in a human, comprising the steps of:
a) administering to the human a composition comprising an antisense oligonucleotide 10 to 30 nucleosides in length, targeted to a HBV nucleic acid (an HBV ASO);
b) administering to the human a i) composition comprising a replication-defective chimpanzee adenoviral (ChAd) vector comprising a polynucleotide encoding a hepatitis B surface antigen (HBs) and a nucleic acid encoding a hepatitis B virus core antigen (HBc) and, concomitantly, ii) a composition comprising a recombinant hepatitis B surface antigen (HBs), a recombinant hepatitis B virus core antigen (HBc) and an adjuvant;
c) administering to the human i) a composition comprising a Modified Vaccinia Virus Ankara (MVA) vector comprising a polynucleotide encoding a HBs antigen and a nucleic acid encoding a HBc antigen and, concomitantly, ii) a composition comprising a recombinant HBs protein antigen, a recombinant HBc protein antigen and an adjuvant;
d) administering to the human i) a composition comprising a MVA vector comprising a polynucleotide encoding a HBs antigen and a nucleic acid encoding a HBc antigen and, concomitantly, ii) a composition comprising a recombinant HBs protein antigen, a recombinant HBc protein antigen and an adjuvant; and
e) administering to the human a i) composition comprising a MVA vector comprising a polynucleotide encoding a HBs antigen and a nucleic add encoding a HBc antigen and, concomitantly, ii) a composition comprising a recombinant HBs protein antigen, a recombinant HBc protein antigen and an adjuvant.

In a particular embodiment, the HBV ASO administered in step a) of the method has 85-95% identity to the sequence GCAGAGGTGAAGCGAAGTGC (SEQ ID NO: 226 of WO2012/145697) complementary to the HBV genome sequence SEQ ID NO: 16 at nucleobases 1583-1602. In a particular embodiment, the HBV ASO administered in step a) of the method has the sequence GCAGAGGTGAAGCGAAGTGC (SEQ ID NO: 226 of WO2012/145697) complementary to the HBV genome sequence SEQ ID NO: 16 at nucleobases 1583-1602.

In certain embodiments, step a) may be repeated. In particular embodiments, step a) is repeated from 2 to 12 times at daily or weekly intervals. In certain embodiments, the period of time between the steps b), c), d) and e) of the method is 2 to 12 weeks, for example 2 weeks, 3 weeks, 4 weeks, 5 weeks, 6 weeks, 7 weeks, 8 weeks, 9 weeks, 10 weeks, 11 weeks or 12 weeks. In one embodiment the period of time between the steps b), c), d) and e) of the method is 4 to 8 weeks. In one embodiment, the period of time between sequential administrations of compositions according to the method is 4 weeks. In one embodiment, the composition i) administered in step b) of the method comprises a ChAd vector selected from the group consisting of ChAd3, ChAd63, ChAd83, ChAd155, ChAd157, Pan 5, Pan 6, Pan 7 (also referred to as C7) and Pan 9, in particular, ChAd63 or ChAd155. In certain embodiments the ChAd vector includes a vector insert encoding HBc and HBs, separated by a sequence encoding the 2A cleaving region of the foot and mouth disease virus. In certain embodiments, the vector insert encodes HBc and HBs, separated by a sequence encoding a spacer which incorporates the 2A cleaving region of the foot and mouth disease virus. In certain embodiments, HBc is fused to hIi. In a particular embodiment, the composition i) administered in step b) of the method comprises a ChAd155 vector which comprises a polynucleotide vector insert encoding hIi, HBc, 2A and HBs, for example, an insert encoding a construct having the structure shown in FIG. 13. In certain embodiments, the vector insert encodes HBc (e.g. SEQ ID NO:11 or an amino acid sequence at least 98% homologous thereto) and HBs (e.g. SEQ ID NO:1 or an amino acid sequence at least 98% homologous thereto), separated by a sequence encoding a spacer which incorporates the 2A cleaving region of the foot and mouth disease virus (e.g. SEQ ID NO:3 or an amino acid sequence at least 98% homologous thereto). In certain embodiments, HBc (e.g. SEQ ID NO:11 or an amino acid sequence at least 98% homologous thereto) is fused to hIi (e.g. SEQ ID NO:7 or an amino acid sequence at least 98% homologous thereto or SEQ ID NO:12 or an amino acid sequence at least 98% homologous thereto). For example, HBc (e.g. SEQ ID NO:11) is fused to hIi (e.g. SEQ ID NO:7), or HBc (e.g. SEQ ID NO:11) is fused to hIi (e.g. SEQ ID NO:12). In one embodiment, the composition i) administered in step b) of the method comprises a ChAd155 vector which comprises a polynucleotide vector insert encoding the amino acid sequence of SEQ ID NO:9. In another embodiment, the composition i) administered in step b) of the method comprises a ChAd155 vector which comprises a polynucleotide vector insert encoding the amino acid sequence of SEQ ID NO:15. In one embodiment, the composition i) administered in step b) of the method comprises a ChAd155 vector which comprises a polynucleotide vector insert having the nucleotide sequence given in SEQ ID NO:10. In another embodiment, the composition i) administered in step b) of the method comprises a ChAd155 vector which comprises a polynucleotide vector insert having the nucleotide sequence given in SEQ ID No:14. In certain embodiments, the composition ii) administered in step b) of the method comprises a full length recombinant hepatitis B surface antigen (HBs), a recombinant hepatitis B virus core antigen (HBc) truncated at the C-terminal, and an adjuvant. In certain embodiments, the truncated recombinant HBc comprises the assembly domain of HBc, for example amino acids 1-149 of HBc. In one embodiment, the composition ii) administered in step b) of the method comprises a full length recombinant HBs (e.g. SEQ ID NO:1), amino acids 1-149 of HBc (e.g. SEQ ID NO:2) and an adjuvant comprising MPL and QS-21. In certain embodiments the recombinant protein HBs and HBc antigens are in the form of virus-like particles.

In one embodiment, the composition i) administered in step c) of the method comprises an MVA vector which includes a vector insert encoding HBc and HBs, separated by a sequence encoding the 2A cleaving region of the foot and mouth disease virus. In certain embodiments, the vector insert encodes HBc (e.g. SEQ ID NO:11 or an amino acid sequence at least 98% homologous thereto) and HBs (e.g. SEQ ID NO:1 or an amino acid sequence at least 98% homologous thereto), separated by a sequence encoding a spacer which incorporates the 2A cleaving region of the foot and mouth disease virus (e.g. SEQ ID NO:3 or an amino acid sequence at least 98% homologous thereto). In a particular embodiment, the composition i) administered in step c) of the method comprises an MVA vector which comprises a polynucleotide vector insert encoding HBc, 2A and HBs, for example, an insert encoding a construct having the structure shown in FIG. 12. In one embodiment, the composition i) administered in step c) of the method comprises an MVA vector which comprises a polynucleotide vector insert encoding the amino acid sequence of SEQ ID NO:5. In one embodiment, the composition i) administered in step c) of the method comprises an MVA vector which comprises a polynucleotide vector insert having the nucleotide sequence given in SEQ ID NO:6. In certain embodiments, the composition ii) administered in step c) of the method comprises a full length recombinant hepatitis B surface antigen (HBs), a recombinant hepatitis B virus core antigen (HBc) truncated at the C-terminal, and an adjuvant. In certain embodiments, the truncated recombinant HBc comprises the assembly domain of HBc, for example amino acids 1-149 of HBc. In one embodiment, the composition ii) administered in step c) of the method comprises a full length recombinant HBs (e.g. SEQ ID NO:1), amino acids 1-149 of HBc (e.g. SEQ ID NO:2) and an adjuvant comprising MPL and QS-21. In certain embodiments the recombinant protein HBs and HBc antigens are in the form of virus-like particles.

In one embodiment, the composition i) administered in step d) of the method comprises an MVA vector which includes a vector insert encoding HBc and HBs, separated by a sequence encoding the 2A cleaving region of the foot and mouth disease virus. In certain embodiments, the vector insert encodes HBc (e.g. SEQ ID NO:11 or an amino acid sequence at least 98% homologous thereto) and HBs (e.g. SEQ ID NO:1 or an amino acid sequence at least 98% homologous thereto), separated by a sequence encoding a spacer which incorporates the 2A cleaving region of the foot and mouth disease virus (e.g. SEQ ID NO:3 or an amino acid sequence at least 98% homologous thereto). In a particular embodiment, the composition i) administered in step d) of the method comprises an MVA vector which comprises a polynucleotide vector insert encoding HBc, 2A and HBs, for example, an insert encoding a construct having the structure shown in FIG. 12. In one embodiment, the composition i) administered in step d) of the method comprises an MVA vector which comprises a polynucleotide vector insert encoding the amino acid sequence of SEQ ID NO:5. In one embodiment, the composition i) administered in step d) of the method comprises an MVA vector which comprises a polynucleotide vector insert having the nucleotide sequence given in SEQ ID NO:6. In certain embodiments, the composition ii) administered in step d) of the method comprises a full length recombinant hepatitis B surface antigen (HBs), a recombinant hepatitis B virus core antigen (HBc) truncated at the C-terminal, and an adjuvant. In certain embodiments, the truncated recombinant HBc comprises the assembly domain of HBc, for example amino acids 1-149 of HBc. In certain embodiments the recombinant protein HBs and HBc antigens are in the form of virus-like particles. In one embodiment, the composition ii) administered in step d) of the method comprises a full length recombinant HBs (e.g. SEQ ID NO:1), amino acids 1-149 of HBc (e.g. SEQ ID NO:2) and an adjuvant comprising MPL and QS-21.

In one embodiment, the composition i) administered in step e) of the method comprises an MVA vector which includes a vector insert encoding HBc and HBs, separated by a sequence encoding the 2A cleaving region of the foot and mouth disease virus. In certain embodiments, the vector insert encodes HBc (e.g. SEQ ID NO:11 or an amino acid sequence at least 98% homologous thereto) and HBs (e.g. SEQ ID NO:1 or an amino acid sequence at least 98% homologous thereto), separated by a sequence encoding a spacer which incorporates the 2A cleaving region of the foot and mouth disease virus (e.g. SEQ ID NO:3 or an amino acid sequence at least 98% homologous thereto). In a particular embodiment, the composition i) administered in step e) of the method comprises an MVA vector which comprises a polynucleotide vector insert encoding HBc, 2A and HBs, for example, an insert encoding a construct having the structure shown in FIG. 12. In one embodiment, the composition i) administered in step e) of the method comprises an MVA vector which comprises a polynucleotide vector insert encoding the amino acid sequence of SEQ ID NO:5. In one embodiment, the composition i) administered in step e) of the method comprises an MVA vector which comprises a polynucleotide vector insert having the nucleotide sequence given in SEQ ID NO:6. In certain embodiments, the composition ii) administered in step e) of the method comprises a full length recombinant hepatitis B surface antigen (HBs), a recombinant hepatitis B virus core antigen (HBc) truncated at the C-terminal, and an adjuvant. In certain embodiments, the truncated recombinant HBc comprises the assembly domain of HBc, for example amino acids 1-149 of HBc. In one embodiment, the composition ii) administered in step e) of the method comprises a full length recombinant HBs (e.g. SEQ ID NO:1), amino acids 1-149 of HBc (e.g. SEQ ID NO:2) and an adjuvant comprising MPL and QS-21. In certain embodiments the recombinant protein HBs and HBc antigens are in the form of virus-like particles.

The present invention also provides a method of inducing a cellular immune response and a humoral immune response in a human with CHB and/or CHD, in particular a CD4+ response and a CD8+ response and an antibody response, the method comprising the steps of:
a) administering to the human a composition comprising an antisense oligonucleotide 10 to 30 nucleosides in length, targeted to a HBV nucleic acid (an HBV ASO);
b) administering to the human a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) administering to the human a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) administering to the human a composition comprising a recombinant hepatitis B surface antigen (HBs), a recombinant hepatitis B virus core antigen (HBc) and an adjuvant.

In one embodiment, the steps of the method are carried out sequentially, with step a) preceding step b), step b) preceding step c) and step c) preceding step d). Optionally, step a) may be repeated. Optionally, step d) may be repeated. In another embodiment, step d) is carried out concomitantly with step b) and/or with step c). In a further embodiment, the method of inducing a cellular immune response and a humoral immune response in a human with CHB and/or CHD, in particular a CD4+ response and a CD8+ response and an antibody response, comprises the steps of:
a) administering to the human a composition comprising an antisense oligonucleotide 10 to 30 nucleosides in length, targeted to a HBV nucleic acid (an HBV ASO);
b) administering to the human i) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc) and, concomitantly, ii) a composition comprising a recombinant hepatitis B surface antigen (HBs), a recombinant hepatitis B virus core antigen (HBc) and an adjuvant; and
c) administering to the human i) a composition comprising a Modified Vaccinia Virus Ankara (MVA) vector comprising a polynucleotide encoding a hepatitis B surface antigen (HBs) and a nucleic acid encoding a hepatitis B virus core antigen (HBc) and, concomitantly, a composition comprising a recombinant hepatitis B surface antigen (HBs), a recombinant hepatitis B virus core antigen (HBc) and an adjuvant.

In one embodiment, the steps of the method are carried out sequentially, with step a) preceding step b) and step b) preceding step c). Optionally, step c) may be repeated.

The present invention also provides a method reducing the level of serum HBsAg and/or the level of serum HBV DNA in a human with CHB and/or CHD, the method comprising the steps of:
a) administering to the human a composition comprising an antisense oligonucleotide 10 to 30 nucleosides in length, targeted to a HBV nucleic acid (an HBV ASO);
b) administering to the human a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) administering to the human a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) administering to the human a composition comprising a recombinant hepatitis B surface antigen (HBs), a recombinant hepatitis B virus core antigen (HBc) and an adjuvant.

In one embodiment, the steps of the method are carried out sequentially, with step a) preceding step b), step b) preceding step c) and step c) preceding step d). Optionally, step a) may be repeated. Optionally, step d) may be repeated. In another embodiment, step d) is carried out concomitantly with step b) and/or with step c).

In a further embodiment, the method of reducing the level of serum HBsAg and/or the level of serum HBV DNA in a human with CHB and/or CHD comprises the steps of:
a) administering to the human a composition comprising an antisense oligonucleotide 10 to 30 nucleosides in length, targeted to a HBV nucleic acid (an HBV ASO);
b) administering to the human i) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc) and, concomitantly, ii) a composition comprising a recombinant hepatitis B surface antigen (HBs), a recombinant hepatitis B virus core antigen (HBc) and an adjuvant; and
c) administering to the human i) a composition comprising a Modified Vaccinia Virus Ankara (MVA) vector comprising a polynucleotide encoding a hepatitis B surface antigen (HBs) and a nucleic acid encoding a hepatitis B virus core antigen (HBc) and, concomitantly, ii) a composition comprising a recombinant hepatitis B surface antigen (HBs), a recombinant hepatitis B virus core antigen (HBc) and an adjuvant.

In one embodiment, the steps of the method are carried out sequentially, with step a) preceding step b) and step b) preceding step c). Optionally, step a) may be repeated. Optionally, step c) may be repeated. In a further embodiment, the level of serum HBsAg is reduced to undetectable levels as determined by quantitative immunoassay. In another embodiment, the level of serum HBV DNA is reduced to undetectable levels as determined by the Cobas® HBV assay or equivalent. In another embodiment, the level of serum HBsAg and/or the level of serum HBV DNA is reduced to and maintained at undetectable levels for at least 6 months. In another embodiment, the level of serum HBsAg and/or the level of serum HBV DNA is reduced to and maintained at undetectable levels and ALT levels are maintained within normal range for at least 6 months.

### Antigens

At least nine genotypes (A through I) of HBV have been identified, differing in their genome by more than 8%. Within a given HBV genotype, multiple geno-subtypes have been identified, differing by 4-8%. The antigens for use in the disclosed methods are suitably selected to provide immunological coverage across multiple, preferably all HBV genotypes. The hepatitis B core protein antigen (HBc) is highly conserved across genotypes and geno-subtypes and the hepatitis B surface protein antigen (HBs) sequence is suitably selected to include key cross-genotype-preserved B-cell epitopes which allow for induction of broad neutralizing responses. Suitably, the sequences of the HBc and of the HBs for use in the disclosed methods and compositions are based upon those from genotype/subtype A2.

Suitably, the HBs antigen for use in the disclosed methods and compositions is derived from the small, middle or large surface antigen protein. In particular, a suitable HBs antigen comprises the small (S) protein of HBV adw2 strain, genotype A. For example, a suitable HBs antigen has the 226 amino acids of amino acid sequence SEQ ID NO:1. When used as recombinant protein, the HBs antigen preferably assembles into virus-like particles. This antigen is included in well-studied marketed hepatitis-B prophylactic vaccines (Engerix B, Fendrix, Twinrix and others), and has been demonstrated to be protective against hepatitis B, across genotypes. Preferably the recombinant HBs protein antigen is expressed from yeast and purified for use in the vaccine compositions and methods of the present invention. Suitable methods for expression and purification are known, for example from EP1307473B1.

The hepatitis B core protein (HBc) is the major component of the nucleocapsid shell packaging the viral genome. This protein (183-185 aa long) is expressed in the cytoplasm of infected cells and remains unglycosylated. HBc comprises a 149 residue assembly domain and a 34-36 residue RNA-binding domain at the C terminus. The HBc antigen for use in the disclosed methods and compositions may be full length or may comprise a C-terminally truncated protein (lacking the RNA-binding C-terminus), for example including 145-149 amino acids of the assembly domain of a wild-type core antigen protein, e.g. amino acids 1-145, 1-146, 1-147, 1-148 or amino acids 1-149 of a wild-type hepatitis B core antigen protein. The truncated protein retains the ability to assemble into nucleocapsid particles. A suitable HBc antigen for use in the disclosed methods and compositions has an amino acid sequence from HBV adw2 strain, genotype A. When used as recombinant protein, the HBc antigen is suitably truncated from the wild-type at the C-terminus, in particular, the antigen may have the amino acid sequence of SEQ ID NO:2. Preferably the recombinant HBc protein antigen is expressed from E. coli and purified for use in the vaccine compositions and methods of the present invention. Methods for recombinant expression of viral proteins in E. coli are well known in the art.

When used as recombinant protein, the HBc antigen preferably assembles into virus-like particles. When expressed from a viral vector, the HBc antigen may be full-length or truncated, for example is suitably a full length HBc antigen (e.g. SEQ ID NO:11). Suitable doses of recombinant HBs antigen for use in the methods disclosed herein are from 10 g per dose to 100 ug per dose, such as 10 ug, 15 ug, 20 ug, 25 ug, 30 ug, 35 ug, 40 ug, 45 ug, 50 ug, 55 ug, 60 ug, 65 ug, 70 ug, 75 ug, 80 ug, 85 ug, 90 ug, 95 ug, or 100 ug per dose. Suitable doses of recombinant HBc antigen for use in the methods disclosed herein are from 10 ug per dose to 100 ug per dose, such as 10 ug, 15 ug, 20 ug, 25 ug, 30 ug, 35 ug, 40 ug, 45 ug, 50 ug, 55 ug, 60 ug, 65 ug, 70 ug, 75 ug, 80 ug, 85 ug, 90 ug, 95 ug, or 100 ug per dose.

Antigens are substances which induce an immune response in the body, especially the production of antibodies. Antigens may be of foreign, i.e. pathogenic, origin or stem from the organism itself, the latter are referred to as self- or auto antigens. Antigens can be presented on the surface of antigen presenting cells by MHC molecules. There are two classes of MHC molecules, MHC class I (MHC-I) and MHC-class-II (MHC-II). The MHC-II molecules are membrane-bound receptors which are synthesized in the endoplasmic reticulum and leave the endoplasmic reticulum in a MHC class II compartment. In order to prevent endogenous peptides, i.e. self-antigens, from binding to the MHC-II molecule and being presented to generate an immune response, the nascent MHC-II molecule combines with another protein, the invariant chain, which blocks the peptide-binding cleft of the MHC-II molecule. The human invariant chain (hIi, also known as CD74 when expressed on the plasma membrane), is an evolutionarily conserved type II membrane protein which has several roles within the cell and throughout the immune system [Borghese, 2011]. When the MHC class II compartment fuses to a late endosome containing phagocytosed and degraded foreign proteins, the invariant chain is cleaved to leave only the CLIP region bound to the MHC-II molecule. In a second step, CLIP is removed by an HLA-DM molecule leaving the MHC-II molecule free to bind fragments of the foreign proteins. Said fragments are presented on the surface of the antigen-presenting cell once the MHC class II compartment fuses with the plasma membrane, thus presenting the foreign antigens to other cells, primarily T-helper cells.

It is known that the immune response against an antigen is increased when an adenovirus expression system encoding a fusion of invariant chain and said antigen is used for vaccination (see WO2007/062656, which also published as US2011/0293704 and is incorporated by reference for the purpose of disclosing invariant chain sequences), i.e. the invariant chain enhances the immunogenicity of the antigen and an invariant chain such as hIi is sometimes referred to as a “genetic adjuvant” in recognition of this effect. Moreover, said adenoviral construct has proven useful for priming an immune response in the context of prime-boosting vaccination regimens (see WO2014/141176, which also published as US2016/0000904; and WO2010/057501, which also published as US2010/0278904 and is incorporated by reference for the purpose of disclosing invariant chain sequences and adenoviral vectors encoding invariant chain sequences). In particular, the hIi sequence and hIi has the potential to increase CD8⁺ T-cell responses [Spencer, 2014; Capone, 2014]. In certain embodiments, a nucleotide sequence included within a vector for use in the methods, uses and compositions disclosed herein may include a nucleotide sequence coding for hIi. The amino acid sequence for hIi as can be included in the disclosed adenoviral vector ChAd155-hIi-HBV is set out in SEQ ID NO:7, and an alternative sequence is set out in SEQ ID NO:12. Nucleotide sequences encoding these amino acid sequences are set out in SEQ ID NO:8 and SEQ ID NO:13. Suitably, a nucleotide sequence coding for hIi is fused to the nucleotide sequence coding for the HBc antigen so as to produce a fusion protein in which an hIi polypeptide is N-terminally fused to the HBc antigen.

### Vectors

In addition to the polynucleotide encoding the antigen proteins (also referred to herein as the “insert”), the vectors for use in the methods and compositions disclosed herein may also include conventional control elements which are operably linked to the encoding polynucleotide in a manner that permits its transcription, translation and/or expression in a cell transfected with the vector. Thus the vector insert polynucleotide which encodes the protein antigens is incorporated into an expression cassette with suitable control elements.

Expression control elements include appropriate transcription initiation, termination, promoter and enhancer sequences; efficient RNA processing signals such as splicing and polyadenylation (poly A) signals including rabbit beta-globin polyA; sequences that stabilize cytoplasmic mRNA; sequences that enhance translation efficiency (e.g., Kozak consensus sequence); sequences that enhance protein stability; and when desired, sequences that enhance secretion of the encoded product.

A promoter is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A great number of expression control sequences, including promoters which are internal, native, constitutive, inducible and/or tissue-specific, are known in the art and may be utilized.

Examples of constitutive promoters include, the TBG promoter, the retroviral Rous sarcoma virus (RSV) LTR promoter (optionally with the RSV enhancer), the cytomegalovirus (CMV) promoter (optionally with the CMV enhancer, see, e.g., Boshart et al, Cell, 41:521-530 (1985)), the CASI promoter, the SV40 promoter, the dihydrofolate reductase promoter, the β-actin promoter, the phosphoglycerol kinase (PGK) promoter, and the EF1a promoter (Invitrogen). Suitably the promoter is an CMV promoter or variant thereof, more suitably a human CMV (HCMV) promoter or variant thereof.

### Adenoviral Vectors

Adenovirus has been widely used for gene transfer applications due to its ability to achieve highly efficient gene transfer in a variety of target tissues and its large transgene capacity. Conventionally, E1 genes of adenovirus are deleted and replaced with a transgene cassette consisting of the promoter of choice, cDNA sequence of the gene of interest and a poly A signal, resulting in a replication defective recombinant virus. Human adenovirus vectors have been shown to be potent vectors for the induction of CD8⁺ T-cell response to transgene, in animal models as well as in humans. Adenoviruses have a broad tropism and have the capability to infect replicating as well as non-replicating cells. The main limitation for clinical application of vectors based of human adenovirus is the high prevalence of neutralizing antibodies in the general population. Adenoviruses isolated from alternative species have been considered as potential vaccine vectors to circumvent the issue of the pre-existing anti-adenovirus immunity in humans. Among them, simian adenoviruses derived from chimpanzees, gorillas or bonobos may be suitable for use in delivering antigens and eliciting a targeted T cell and/or humoral response to those antigens in humans. Simian adenoviruses including those derived from chimpanzees have been tested in clinical research. Chimpanzee adenoviral vectors have low/no seroprevalence in the human population, are not known to cause pathological illness in humans and some ChAd vectors can be grown to high titres in cell lines previously used for production of clinical-grade material such as human embryonic kidney cells 293 (HEK 293).

A replication-incompetent or replication-defective adenovirus is an adenovirus which is incapable of replication because it has been engineered to comprise at least a functional deletion (or “loss-of-function” mutation), i.e. a deletion or mutation which impairs the function of a gene without removing it entirely, e.g. introduction of artificial stop codons, deletion or mutation of active sites or interaction domains, mutation or deletion of a regulatory sequence of a gene etc, or a complete removal of a gene encoding a gene product that is essential for viral replication, such as one or more of the adenoviral genes selected from E1A, E1B, E2A, E2B, E3 and E4 (such as E3 ORF1, E3 ORF2, E3 ORF3, E3 ORF4, E3 ORF5, E3 ORF6, E3 ORF7, E3 ORF8, E3 ORF9, E4 ORF7, E4 ORF6, E4 ORF5, E4 ORF4, E4 ORF3, E4 ORF2 and/or E4 ORF1). Suitably the E1 and E3 genes are deleted. More suitably the E1, E3 and E4 genes are deleted.

Suitable vectors for use in the methods and compositions disclosed herein are replication-defective chimpanzee adenoviral vectors, for example ChAd3, ChAd63, ChAd83, ChAd155, ChAd157, Pan 5, Pan 6, Pan 7 (also referred to as C7) or Pan 9. Examples of such strains are described in WO03/000283, WO2005/071093, WO2010/086189 and WO2016/198621. The ChAd155 vector (see WO2016/198621 which is incorporated by reference for the purpose of disclosing ChAd155 vector sequences and methods) belongs to the same phylogenetic adenovirus group as the ChAd3 vector (group C). In one embodiment, a vector for use in the methods and compositions disclosed herein is a ChAd vector of phylogenetic group C, for example ChAd3 or ChAd155. In one specific embodiment, a method of treating chronic hepatitis B disclosed herein comprises the step of administering to a human a composition comprising a ChAd155 vector comprising a polynucleotide encoding a hepatitis B surface antigen (HBs) and a nucleic add encoding a hepatitis B virus core antigen (HBc). A suitable dose of a ChAd vector for use in the methods disclosed herein is 1×10⁸-1×10¹¹ viral particles (vp) per dose, for example about 1×10⁸, 5×10⁸, 1×10⁹, 5×10⁹, 1×10¹⁰, 5×10¹⁰ or 1×10¹¹ viral particles (vp) per dose.

More specifically, in one embodiment a vector for use in the methods and compositions disclosed herein is a replication-defective Chimpanzee Adenovirus vector ChAd155 encoding a fusion of sequences derived from two HBV proteins: HBc (core, nucleocapsid protein) and HBs (small surface antigen). In certain specific embodiments, the vector is ChAd155 encoding HBc and HBs, separated by SEQ ID NO:3, a spacer which incorporates a sequence encoding the 2A cleaving region of the foot and mouth disease virus (FMDV) [Donnelly et al. 2001] (resulting in a 23 amino acid tail at C-terminal of the upstream protein and a single proline at the N-terminal of the downstream protein), for processing of the HBc and HBs into separate proteins. Cleavage of the core from the surface antigens permits proper folding of HBs, allowing generation of an antibody response to the surface antigen. Alternatively, the adenoviral vector may be a dual-promoter (bi-cistronic) vector to allow independent expression of the HBs and HBc antigens.

In certain embodiments, the N-terminal part of the gene encoding the HBc protein may be fused to the gene encoding the human Major Histocompatibility Complex (MHC) class II-associated Invariant chain, p35 isoform (i.e. hIi or CD74). Thus, a particular ChAd155 vector for use in the methods and compositions disclosed herein comprises a polynucleotide vector insert encoding a construct having the structure shown in FIG. 13, comprising hIi, HBc, 2A and HBs. The amino acid sequence of such a construct is given in SEQ ID NO:9 and a nucleotide sequence encoding the amino acid sequence of the construct is given in SEQ ID NO:10. The amino acid sequence of an alternative such construct is given in SEQ ID NO:15 and a nucleotide sequence encoding the amino acid sequence of the construct is given in SEQ ID NO:14.

### Modified Vaccinia Virus Ankara (MVA) Vector

Modified Vaccinia Virus Ankara (MVA), replication-deficient in humans and other mammals, is derived from the vaccinia virus. It belongs to the poxvirus family and was initially developed to improve the safety of smallpox vaccination by passage of vaccinia virus over 570 times in chicken embryo fibroblast (CEF) cells, resulting in multiple deletions after which the virus was highly attenuated and replication-deficient in humans and other mammals. The replication defect occurs at a late stage of virion assembly such that viral and recombinant gene expression is unimpaired, making MVA an efficient single round expression vector incapable of causing infection in mammals. MVA has subsequently been extensively used as a viral vector to induce antigen-specific immunity against transgenes, both in animal models and in humans. A description of MVA can be found in Mayr A, et. al. (1978) and in Mayr, A., et. al. (1975).

In one embodiment, MVA is derived from the virus seed batch 460 MG obtained from 571th passage of Vaccinia Virus on CEF cells. In another embodiment, MVA is derived from the virus seed batch MVA 476 MG/14/78. In a further embodiment, MVA is derived or produced prior to 31 Dec. 1978 and is free of prion contamination. A suitable dose of a MVA vector for use in the methods disclosed herein is 1×10⁶-1×10⁹ plaque forming units (pfu) per dose, for example about 1×10⁶, 2×10⁶, 5×10⁶, 1×10⁷, 2×10⁷, 5×10⁷, 1×10⁸, 2×10⁸, 5×10⁸ or 1×10⁹ pfu per dose.

In one specific embodiment, a method of treating chronic hepatitis B disclosed herein comprises the step of administering to a human a composition comprising a MVA vector comprising a polynucleotide encoding a hepatitis B surface antigen (HBs) and a nucleic acid encoding a hepatitis B virus core antigen (HBc).

More specifically, in one embodiment a vector for use in the methods and compositions disclosed herein is MVA encoding a fusion of sequences derived from two HBV proteins: HBc (core nucleocapsid protein) and HBs (small surface antigen). In certain embodiments, a vector for use in the methods and compositions disclosed herein is MVA encoding HBc and HBs, separated by SEQ ID NO:3, a spacer which incorporates a sequence encoding the 2A cleaving region of the foot and mouth disease virus (resulting in a 23 amino acid tail at the C-terminal of the upstream protein and a single proline at the N-terminal of the downstream protein), for processing of the HBc and HBs into separate proteins. Thus, a particular MVA vector for use in the methods and compositions disclosed herein comprises a polynucleotide vector insert encoding a construct having the structure shown in FIG. 12, comprising HBc, 2A and HBs. The amino acid sequence of such a construct is given in SEQ ID NO:5 and a nucleotide sequence encoding the amino acid insert construct is given in SEQ ID NO:6.

### Antisense Oligonucleotides (ASO)

For a cell to express the protein coded by the DNA, one strand of the DNA serves as a template for the synthesis of a complementary strand of RNA. The template DNA strand is called the transcribed strand and its sequence is antisense, or complementary, to the mRNA transcript, which has the same sequence as the sense sequence of the original double-stranded DNA. Because the DNA is double-stranded, the strand complementary to the antisense sequence is called the non-transcribed strand, or sense strand, and has the same sequence as the mRNA transcript (except T nucleobases in the DNA sequence are substituted with U nucleobases in the RNA sequence).

A nucleic acid that is complementary to the RNA transcribed from the DNA is termed an “anti-sense” oligonucleotide (ASO) because its base sequence is complementary to the gene's messenger RNA (mRNA)—the “sense” sequence. Thus, a coding DNA region having a sense sequence of 5′-AAGGTC-3″ will be transcribed to produce a mRNA having a sense sequence of 5′-AAGGUC-3′ and so an antisense oligomer to that sense sequence will have a sequence of 3′-UUCCAG-5′ if it comprises RNA nucleobases, or 3′-TTCCAG-5′ if the antisense oligomer comprises DNA nucleobases.

Currently, a main focus of antisense therapy involves the use of an oligomer or oligonucleotide, approximately 20 nucleotide/nucleosides in length, synthesized to be complementary to the specific “sense” (5′ to 3′orientation) DNA or mRNA sequence responsible for expression or translation of a targeted protein.

Once introduced into a cell, the antisense oligonucleotide hybridizes to its corresponding mRNA sequence through Watson-Crick binding, forming a heteroduplex. Once a duplex is formed, translation of the protein coded by the sequence of bound mRNA is inhibited. Antisense therapy can therefore directly target the RNA transcripts for antigens and thereby reduce serum HBeAg and HBsAg levels. Because of the multiple, overlapping transcripts produced upon HBV infection, there is also an opportunity for a single antisense oligomer to reduce HBV DNA more than one HBV antigen.

There are several mechanisms proposed through which the oligonucleotide/mRNA duplex may hinder subsequent translation. The most widely accepted explanation involves the degradation of the mRNA in the heteroduplex by the ubiquitous enzyme RNase H. RNase H is attracted to the heteroduplex and cleaves the bound mRNA, while leaving the antisense oligonucleotide (ASO) sequence intact, allowing the ASO to continue seeking and binding to corresponding mRNA sequences. Some other accepted explanations of translation inhibition through antisense therapy which may occur separately or in conjunction with RNase H activity include, but are not limited to, the blocking of appropriate ribosome assembly that disables the ribosomal complexes ability to translate, blocking of RNA splicing, and/or impeding appropriate exportation of mRNA.

In the field of antisense therapy, the introduction of chemically modified nucleosides into nucleic acid molecules, particularly into RNA, provides a powerful tool in overcoming potential limitations of in vivo stability and bioavailability inherent to exogenous RNA. For example, the use of chemically modified nucleic acid molecules can enable a lower dose of a particular nucleic acid molecule for a given therapeutic effect, since chemically modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically modified nucleic acid molecule is reduced as compared to a native nucleic acid molecule, for example when compared to an all RNA nucleic acid molecule, the overall activity of the modified nucleic acid molecule can be greater than the native molecule due to improved stability and/or delivery of the molecule.

One useful chemical modification, termed a locked nucleic acid (LNA), introduces a 2′O-4′C-alkylene bridge wherein the alkylene bridge is a C₁₋₆ alkylene bridge, more particularly, a 2′O-4′C-methylene bridge, at one or more RNA or DNA nucleoside moiety. When LNAs are incorporated into antisense RNA or DNA oligomers they have been shown to greatly increase the stability of the antisense RNA or DNA molecule, and thus to greatly increase bioavailability of the antisense RNA or DNA once it is taken up by the host cell. Other useful chemical modifications that can be introduced into the antisense RNA or DNA oligomers to increase stability and bioavailability of the antisense oligomer include phosphorothioate bonds, or phosphotriester bonds, substituted in place of naturally occurring phosphodiester bonds between the individual RNA or DNA nucleotides.

In certain embodiments, a composition comprising an antisense oligonucleotide 10 to 30 nucleosides in length, targeted to a HBV nucleic add (an HBV ASO) for use in the methods, regimens and immunological combinations of the present invention, comprises an HBV ASO which is a modified antisense oligonucleotide. In a particular embodiment, the HBV ASO has 85-95% identity to the sequence GCAGAGGTGAAGCGAAGTGC (SEQ ID NO: 226 of WO2012/145697) complementary to the HBV genome sequence SEQ ID NO: 16 at nucleobases 1583-1602. In a particular embodiment, the HBV ASO has the sequence GCAGAGGTGAAGCGAAGTGC (SEQ ID NO: 226 of WO2012/145697) complementary to the HBV genome sequence SEQ ID NO: 16 at nucleobases 1583-1602.

In certain embodiments, at least one internucleoside linkage of the modified antisense oligonucleotide is a modified internucleoside linkage. In certain embodiments, the at least one modified internucleoside linkage is selected from a phosphotriester internucleoside linkage and a phosphorothioate internucleoside linkage. In certain embodiments, each internucleoside linkage is selected from a phosphotriester internucleoside linkage and a phosphorothioate internucleoside linkage. In certain embodiments, each internucleoside linkage is a phosphorothioate internucleoside linkage.

In certain embodiments, at least one nucleoside of the modified antisense oligonucleotide comprises a modified sugar. In certain embodiments, at least one modified sugar comprises a 2′-O-methoxyethyl group (2′-O(CH₂)₂—OCH₃). In certain embodiments, the modified sugar comprises a 2′-O—CH₃ group,

In certain embodiments, at least one modified sugar is a bicyclic sugar. In certain embodiments, at least one modified sugar the bicyclic sugar comprises a 4′-(CH₂)ₙ—O-2′ bridge, wherein n is 1 or 2. In certain embodiments, the bicyclic sugar comprises a 4′-CH₂-O-2′ bridge. In certain embodiments, the bicyclic sugar comprises a 4′-CH(CH₃)—O-2′ bridge.

In certain embodiments, at least one nucleoside of the modified antisense oligonucleotide comprises a modified nucleobase. In certain embodiments, the modified nucleobase is a 5-methylcytosine.

In certain embodiments, the modified oligonucleotide consists of a single-stranded modified oligonucleotide.

In certain embodiments, the modified antisense oligonucleotide comprises: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar.

In certain embodiments, the modified antisense oligonucleotide consists of 15-30, for example 15, 16, 17, 20, 25 or 30 linked nucleosides, the gap segment consisting of 7 to 15, for example 7, 8, 9, 10, 12 or 15 linked deoxynucleosides, the 5′ wing segment consisting of 3-8, for example 3, 5, 7 or 8 linked nucleosides, the 3′ wing segment consisting of 3-8, for example 3, 5, 7 or 8 linked nucleosides, wherein each nucleoside of each wing segment comprises a 2′-O-methoxyethyl sugar, at least one internucleoside linkage is a phosphorothioate linkage and at least one cytosine is a 5-methylcytosine.

In certain embodiments, the modified antisense oligonucleotide consists of 15-30, for example 15, 16, 17, 20, 25 or 30 linked nucleosides, the gap segment consisting of 7 to 15, for example 7, 8, 9, 10, 12 or 15 linked deoxynucleosides, the 5′ wing segment consisting of 3-8, for example 3, 5, 7 or 8 linked nucleosides, the 3′ wing segment consisting of 3-8, for example 3, 5, 7 or 8 linked nucleosides, wherein at least one nucleoside of each wing segment comprises a 2′-O-methoxyethyl sugar, at least one internucleoside linkage is a phosphorothioate linkage and at each cytosine is a 5-methylcytosine.

In certain embodiments, the modified antisense oligonucleotide consists of 15-30, for example 15, 16, 17, 20, 25 or 30 linked nucleosides, the gap segment consisting of 7 to 15, for example 7, 8, 9, 10, 12 or 15 linked deoxynucleosides, the 5′ wing segment consisting of 3-8, for example 3, 5, 7 or 8 linked nucleosides, the 3′ wing segment consisting of 3-8, for example 3, 5, 7 or 8 linked nucleosides, wherein at least one nucleoside of each wing segment comprises a 2′-O-methoxyethyl sugar, each internucleoside linkage is a phosphorothioate linkage and at least one cytosine is a 5-methylcytosine.

In certain embodiments, the modified antisense oligonucleotide consists of 15-30, for example 15, 16, 17, 20, 25 or 30 linked nucleosides, the gap segment consisting of 7 to 15, for example 7, 8, 9, 10, 12 or 15 linked deoxynucleosides, the 5′ wing segment consisting of 3-8, for example 3, 5, 7 or 8 linked nucleosides, the 3′ wing segment consisting of 3-8, for example 3, 5, 7 or 8 linked nucleosides, wherein each nucleoside of each wing segment comprises a 2′-O-methoxyethyl sugar, each internucleoside linkage is a phosphorothioate linkage and each cytosine is a 5-methylcytosine.

In certain embodiments, the modified antisense oligonucleotide consists of 20 linked nucleosides, the gap segment consisting of ten linked deoxynucleosides, the 5′ wing segment consisting of five linked nucleosides, the 3′ wing segment consisting of five linked nucleosides, each nucleoside of each wing segment comprises a 2′-O-methoxyethyl sugar, each internucleoside linkage is a phosphorothioate linkage and each cytosine is a 5-methylcytosine.

In a particular embodiment, the antisense oligonucleotide targeted to a HBV nucleic acid is a modified oligonucleotide “gapmer” consisting of 20 linked nucleosides in which each internucleoside linkage is a phosphorothioate linkage and each cytosine is a 5-methylcytosine, having the sequence GCAGAGGTGAAGCGAAGTGC (SEQ ID NO: 226 of WO2012/145697) consisting of a 5′ wing segment consisting of five linked nucleosides GCAGA each comprising a 2′-O-methoxyethyl sugar, followed by ten linked deoxynucleosides GGTGAAGCGA and a 3′ wing segment consisting of five linked nucleosides AGTGC each comprising a 2′-O-methoxyethyl sugar.

In certain embodiments, the antisense compound may be covalently linked to one or more moieties or conjugates which enhance the activity, cellular distribution or cellular uptake of the resulting antisense oligonucleotides. Typical conjugate groups include cholesterol moieties, lipid moieties and carbohydrates. In certain embodiments, the conjugate group is a carbohydrate. In particular embodiments, the conjugate group is a sugar. In particular embodiments, the conjugate group is a carbohydrate which comprises an asialoglycoprotein receptor (ASGPR) binding moiety such as an N-acetylgalactosamine (GalNAc) sugar. In certain embodiments, the conjugate group carbohydrate is a GalNAc sugar comprising:

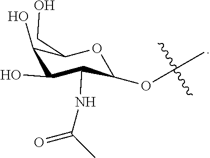

In certain embodiments, the antisense oligonucleotide comprises a modified oligonucleotide, e.g. a gapmer as described above, of SEQ ID NO: 226 (GCAGAGGTGAAGCGAAGTGC) of WO2012/0145697, conjugated to a carbohydrate group having the structure:

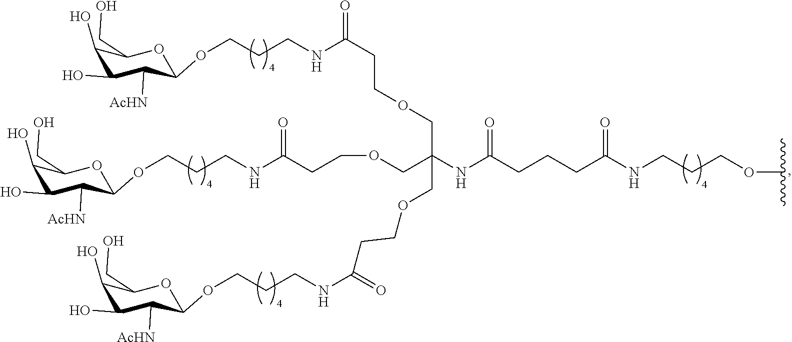

or a pharmaceutically acceptable salt thereof (wherein the salt is an H₂SO₄ salt or an HCl salt).

In certain embodiments, the antisense oligonucleotide is a modified oligonucleotide consisting of 20 linked nucleosides having a nucleobase sequence consisting of SEQ ID NO: 226 of WO2012/0145697, and wherein the modified oligonucleotide comprises:
a gap segment consisting of ten linked deoxynucleosides;
a 5′ wing segment consisting of five linked nucleosides;
a 3′ wing segment consisting of five linked nucleosides;

wherein the gap segment is positioned between the 5′ wing segment and the 3′ wing segment, wherein each nucleoside of each wing segment comprises a 2′-O-methoxyethyl sugar, wherein each cytosine residue is a 5-methylcytosine, and wherein each internucleoside linkage of the modified oligonucleotide is a phosphorothioate linkage.

In a certain embodiment the antisense oligonucleotide has the structure:

or a pharmaceutically acceptable salt thereof (wherein the salt is an H₂SO₄ salt or an HCl salt).

In certain embodiments the antisense oligonucleotide comprises a modified antisense oligonucleotide and a conjugate group, wherein the modified antisense oligonucleotide consists of 12 to 30 linked nucleosides and comprises a nucleobase sequence comprising a portion of at least 8 contiguous nucleobases complementary to an equal length portion of SEQ ID NO: 16 (GENBANK Accession No. U95551.1), wherein the nucleobase sequence of the modified oligonucleotide is at least 80% complementary to a 12 to 30 nucleotide fragment of SEQ ID NO: 16; and wherein the conjugate group comprises:

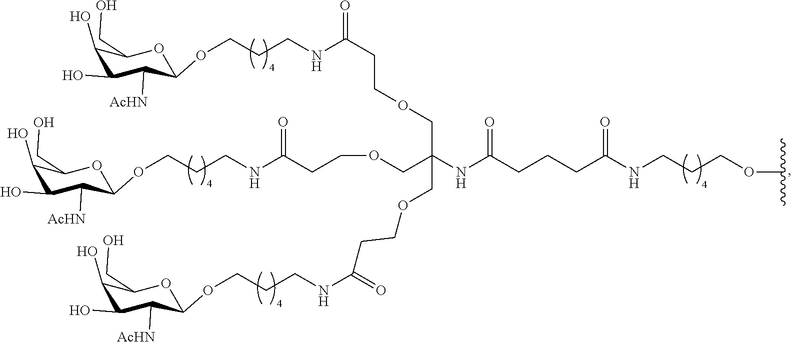

In certain embodiments, the modified antisense oligonucleotide comprises at least one modified sugar wherein the modified sugar is selected from a 2′-O-methoxyethyl a 2′-O-methoxyethyl, a constrained ethyl, a 3′-fluoro-HNA and a bicyclic sugar.

In certain embodiments, the at least one modified sugar is 2′-O-methoxyethyl and the modified antisense oligonucleotide further comprises a bicyclic sugar that comprises a 4′-(CH₂)ₙ—O-2′ bridge, wherein n is 1 or 2.

In certain embodiments, the least one nucleoside of the modified antisense oligonucleotide comprises a modified nucleobase, wherein the at least one nucleoside comprises a modified nucleobase, wherein the modified nucleobase is a 5-methylcytosine.

In certain embodiments, the conjugate group is linked to the modified antisense oligonucleotide at the 5′ end of the modified antisense oligonucleotide, or the conjugate group is linked to the 3′-end of the modified antisense oligonucleotide.

In certain embodiments, each internucleoside linkage of the modified antisense oligonucleotide is selected from a phosphodiester internucleoside linkage, a phosphotriester internucleoside linkage and a phosphorothioate internucleoside linkage.

In certain embodiments, each internucleoside linkage of the modified antisense oligonucleotide is selected from a phosphodiester internucleoside linkage and a phosphorothioate internucleoside linkage.

In certain embodiments the modified oligonucleotide is single-stranded.

### Pharmaceutical Compositions

In certain embodiments, the composition comprising a replication-defective chimpanzee adenoviral vector for use in a method of treating CHB and/or CHD comprises a ChAd vector selected from the group consisting of ChAd3, ChAd63, ChAd83, ChAd155, ChAd157, Pan 5, Pan 6, Pan 7 (also referred to as C7) and Pan 9, in particular, ChAd63 or ChAd155. In certain embodiments the ChAd vector includes a vector insert encoding HBc and HBs, separated by a sequence encoding the 2A cleaving region of the foot and mouth disease virus. In certain embodiments, the vector insert encodes HBc (e.g. SEQ ID NO:11 or an amino acid sequence at least 98% homologous thereto) and HBs (e.g. SEQ ID NO:1 or an amino acid sequence at least 98% homologous thereto), separated by a sequence encoding a spacer which incorporates the 2A cleaving region of the foot and mouth disease virus (e.g. SEQ ID NO:3 or an amino acid sequence at least 98% homologous thereto). In certain embodiments, HBc (e.g. SEQ ID NO:11 or an amino acid sequence at least 98% homologous thereto) is fused to hIi (e.g. SEQ ID NO:7 or an amino acid sequence at least 98% homologous thereto or SEQ ID NO:12 or an amino acid sequence at least 98% homologous thereto). For example, HBc (e.g. SEQ ID NO:11) is fused to hIi (e.g. SEQ ID NO:7), or HBc (e.g. SEQ ID NO:11) is fused to hIi (e.g. SEQ ID NO:12). In a particular embodiment, the composition comprising a replication-defective chimpanzee adenoviral vector for use in a method of treating CHB and/or CHD comprises a ChAd155 vector which comprises a polynucleotide vector insert encoding hIi, HBc, 2A and HBs, for example, an insert encoding a construct having the structure shown in FIG. 13. In one embodiment, the composition comprising a replication-defective chimpanzee adenoviral vector for use in a method of treating CHB and/or CHD comprises a ChAd vector which comprises a polynucleotide vector insert encoding the amino acid sequence of SEQ ID NO:9 or the amino acid sequence of SEQ ID NO:15. In certain embodiments, the composition comprising a replication-defective chimpanzee adenoviral vector for use in a method of treating CHB and/or CHD comprises a ChAd vector which comprises a polynucleotide vector insert having the nucleotide sequence given in SEQ ID NO:10 or the nucleotide sequence given in SEQ ID NO:14. In one specific embodiment, the vector is a ChAd155 vector. Thus, in certain embodiments, the composition comprising a replication-defective chimpanzee adenoviral vector for use in a method of treating CHB and/or CHD comprises a ChAd155 vector which comprises a polynucleotide vector insert encoding the amino acid sequence of SEQ ID NO:9. In other embodiments, the composition comprising a replication-defective chimpanzee adenoviral vector for use in a method of treating CHB and/or CHD comprises a ChAd155 vector which comprises a polynucleotide vector insert encoding the amino acid sequence of SEQ ID NO:15. In one embodiment, the composition comprising a replication-defective chimpanzee adenoviral vector for use in a method of treating CHB and/or CHD comprises a ChAd155 vector which comprises a polynucleotide vector insert having the nucleotide sequence given in SEQ ID NO:10. In other embodiments, the composition comprising a replication-defective chimpanzee adenoviral vector for use in a method of treating CHB and/or CHD comprises a ChAd155 vector which comprises a polynucleotide vector insert having the nucleotide sequence given in SEQ ID NO:14.

In one embodiment, the composition comprising a MVA vector for use in a method of treating CHB and/or CHD comprises an MVA vector which includes a vector insert encoding HBc and HBs, separated by a sequence encoding the 2A cleaving region of the foot and mouth disease virus. In certain embodiments, the vector insert encodes HBc and HBs, separated by a sequence encoding a spacer which incorporates the 2A cleaving region of the foot and mouth disease virus. In a particular embodiment, the composition comprising a MVA vector for use in a method of treating CHB and/or CHD comprises an MVA vector which comprises a polynucleotide vector insert encoding HBc, 2A and HBs, for example, an insert encoding a construct having the structure shown in FIG. 12. In certain embodiments, the vector insert encodes HBc (e.g. SEQ ID NO:11 or an amino acid sequence at least 98% homologous thereto) and HBs (e.g. SEQ ID NO:1 or an amino acid sequence at least 98% homologous thereto), separated by a sequence encoding a spacer which incorporates the 2A cleaving region of the foot and mouth disease virus (e.g. SEQ ID NO:3 or an amino acid sequence at least 98% homologous thereto). In one embodiment, the composition comprising a MVA vector for use in a method of treating CHB and/or CHD comprises an MVA vector which comprises a polynucleotide vector insert encoding the amino acid sequence of SEQ ID NO:5. In one embodiment, the composition comprising a MVA vector for use in a method of treating CHB and/or CHD comprises an MVA vector which comprises a polynucleotide vector insert having the nucleotide sequence given in SEQ ID NO:6.

In one embodiment, the composition comprising a recombinant HBs antigen, a recombinant HBc antigen and an adjuvant for use in a method of treating CHB and/or CHD comprises recombinant HBc and recombinant HBs in a 1:1 ratio. In another embodiment the ratio of HBc to HBs in the composition is greater than 1, for example the ratio of HBc to HBs may be 1,5:1, 2:1, 2.5:1, 3:1, 3.5:1, 4:1, 4.5:1, 5:1, 5.5:1, 6:1 or more, especially 3:1 to 5:1, such as 3:1, 4:1 or 5:1, particularly a ratio of 4:1. In particular embodiments, the composition comprising a recombinant HBs antigen, a recombinant HBc antigen and an adjuvant for use in a method of treating CHB and/or CHD comprises recombinant HBc and recombinant HBs in a ratio of 4:1 or more. In certain embodiments, the composition comprising a recombinant HBs antigen, a recombinant HBc antigen and an adjuvant for use in a method of treating CHB and/or CHD comprises a full length recombinant hepatitis B surface antigen (HBs) (e.g. SEQ ID NO:1), a recombinant hepatitis B virus core antigen (HBc) truncated at the C-terminal, and an adjuvant. In certain embodiments, the truncated recombinant HBc comprises the assembly domain of HBc, for example amino acids 1-149 of HBc (e.g. SEQ ID NO:2). In one embodiment, the composition comprising a recombinant HBs antigen, a recombinant HBc antigen and an adjuvant for use in a method of treating CHB and/or CHD comprises a full length recombinant HBs, amino acids 1-149 of HBc and an adjuvant comprising MPL and QS-21. For example, the composition comprising a recombinant HBs antigen, a recombinant HBc antigen and an adjuvant for use in a method of treating CHB and/or CHD comprises a full length recombinant HBs (SEQ ID NO: 1), amino acids 1-149 of HBc (SEQ ID NO: 2) and an adjuvant comprising MPL and QS-21. In certain embodiments the recombinant protein HBs and HBc antigens are in the form of virus-like particles.

The compositions disclosed herein, which find use in the disclosed methods, are suitably pharmaceutically acceptable compositions. Suitably, a pharmaceutical composition will include a pharmaceutically acceptable carrier or diluent. In certain embodiments, the compositions comprise a salt of a modified oligonucleotide.

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense oligonucleotide targeted to a HBV nucleic acid can be utilized in pharmaceutical compositions by combining the ASO with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising HBV ASO and a pharmaceutically acceptable diluent. In certain embodiments, the pharmaceutically acceptable diluent is PBS. The compositions which comprise an HBV ASO may be prepared for administration by suspension of the ASO, or a pharmaceutically acceptable salt thereof, in PBS or any pharmaceutically or physiologically acceptable carrier such as isotonic saline, water for injection, or other suitable diluent.

The compositions which comprise ChAd or MVA vectors may be prepared for administration by suspension of the viral vector particles in a pharmaceutically or physiologically acceptable carrier such as isotonic saline or other isotonic salts solution. The appropriate carrier will be evident to those skilled in the art and will depend in large part upon the route of administration.

The compositions which comprise recombinant protein antigens may be prepared by isolation and purification of the proteins from the cell culture in which they are expressed, suspension in a formulation buffer which includes one or more salts, surfactants and/or cryoprotectants, and lyophilized. For example, a suitable formulation buffer may include a sugar, or a mixture of sugars e.g. sucrose, trehalose or sucralose as a cryoprotectant and a non-ionic copolymer e.g. a poloxamer as a surfactant. For administration, lyophilised recombinant protein formulations are reconstituted in a pharmaceutically or physiologically acceptable carrier such as isotonic saline or other isotonic salts solution for injection or inhalation. The appropriate carrier will be evident to those skilled in the art and will depend in large part upon the route of administration. The reconstituted composition may also include an adjuvant or mixture of adjuvants, in one embodiment, the lyophilised recombinant proteins are reconstituted in a liquid adjuvant system formulation.

The term “carrier”, as used herein, refers to a pharmacologically inactive substance such as but not limited to a diluent, excipient, or vehicle with which the therapeutically active ingredient is administered. Liquid carriers include but are not limited to sterile liquids, such as saline solutions in water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions, Examples of suitable pharmaceutical carriers are described in “Remington's Pharmaceutical Sciences” by E. W. Martin.

Compositions for use in the methods disclosed herein may include, in addition to the ASO, vector or recombinant proteins of the composition, an adjuvant system. The term “adjuvant” refers to an agent that augments, stimulates, activates, potentiates, or modulates the immune response to an antigen of the composition at either the cellular or humoral level, e.g. immunologic adjuvants stimulate the response of the immune system to the antigen(s), but have no immunological effect by themselves. The compositions disclosed herein may include an adjuvant as a separate ingredient in the formulation, whether or not a vector comprised in the composition also encodes a “genetic adjuvant” such as hIi.

Suitable adjuvants are those which can enhance the immune response in subjects with chronic conditions and subverted immune competence. CHB patients are characterised by their inability to mount an efficient innate and adaptive immune response to the virus, which rends efficient vaccine development challenging. In these patients, one key function of an adjuvanted vaccine formulation should aim to direct the cell-mediated immune response towards a T Helper 1 (Th1) profile recognised to be critical for the removal of intracellular pathogens.

Examples of suitable adjuvants include but are not limited to inorganic adjuvants (e.g. inorganic metal salts such as aluminium phosphate or aluminium hydroxide), organic non-peptide adjuvants (e.g. saponins, such as QS21, or squalene), oil-based adjuvants (e.g. Freund's complete adjuvant and Freund's incomplete adjuvant), cytokines (e.g. IL-1β, IL-2, IL-7, IL-12, IL-18, GM-CFS, and IFN-γ) particulate adjuvants (e.g. immuno-stimulatory complexes (ISCOMS), liposomes, or biodegradable microspheres), virosomes, bacterial adjuvants (e.g. monophosphoryl lipid A (MPL), such as 3-de-O-acylated monophosphoryl lipid A (3D-MPL), or muramyl peptides), synthetic adjuvants (e.g. non-ionic block copolymers, muramyl peptide analogues, or synthetic lipid A), synthetic polynucleotides adjuvants (e.g. polyarginine or polylysine) and immunostimulatory oligonucleotides containing unmethylated CpG dinucleotides (“CpG”). In particular, the adjuvant(s) may be organic non-peptide adjuvants (e.g. saponins, such as QS21, or squalene) and/or bacterial adjuvants (e.g. monophosphoryl lipid A (MPL), such as 3-de-O-acylated monophosphoryl lipid A (3D-MPL)

One suitable adjuvant is monophosphoryl lipid A (MPL), in particular 3-de-O-acylated monophosphoryl lipid A (3D-MPL). Chemically it is often supplied as a mixture of 3-de-O-acylated monophosphoryl lipid A with either 4, 5, or 6 acylated chains. It can be purified and prepared by the methods taught in GB 21222048, which reference also discloses the preparation of diphosphoryl lipid A, and 3-O-deacylated variants thereof. Other purified and synthetic lipopolysaccharides have been described [U.S. Pat. No. 6,005,099 and EP072947381; Hilgers, 1986; Hilgers, 1987; and EP0549074B1].

Saponins are also suitable adjuvants [Lacaille-Dubois, 1996]. For example, the saponin Quil A (derived from the bark of the South American tree Quillaja saponaria Molina), and fractions thereof, are described in U.S. Pat. No. 5,057,540 and Kensil, 1996; and EP 0 362 279 B1. Purified fractions of Quil A are also known as immunostimulants, such as QS21 and QS17; methods of their production are disclosed in U.S. Pat. No. 5,057,540 and EP 0 362 279 B1. Use of QS21 is further described in Kensil, 1991. Combinations of QS21 and polysorbate or cyclodextrin are also known (WO 99/10008). Particulate adjuvant systems comprising fractions of QuilA, such as QS21 and QS7 are described in WO 96/33739 and WO 96/11711.

Adjuvants such as those described above may be formulated together with carriers, such as liposomes, oil in water emulsions, and/or metallic salts (including aluminum salts such as aluminum hydroxide). For example, 3D-MPL may be formulated with aluminum hydroxide (EP 0 689 454) or oil in water emulsions (WO 95/17210); QS21 may be formulated with cholesterol containing liposomes (WO 96/33739), oil in water emulsions (WO 95/17210) or alum (WO 98/15287).

Combinations of adjuvants may be utilized in the disclosed compositions, in particular a combination of a monophosphoryl lipid A and a saponin derivative (see, e.g., WO 94/00153; WO 95/17210; WO 96/33739; WO 98/56414; WO 99/12565; WO 99/11241), more particularly the combination of QS21 and 3D-MPL as disclosed in WO 94/00153, or a composition where the QS21 is quenched in cholesterol-containing liposomes (DQ) as disclosed in WO 96/33739. A potent adjuvant formulation involving QS21, 3D-MPL & tocopherol in an oil in water emulsion is described in WO 95/17210 and is another formulation which may find use in the disclosed compositions. Thus, suitable adjuvant systems include, for example, a combination of monophosphoryl lipid A, preferably 3D-MPL, together with an aluminium salt (e.g. as described in WO00/23105). A further exemplary adjuvant comprises QS21 and/or MPL and/or CpG. QS21 may be quenched in cholesterol-containing liposomes as disclosed in WO 96/33739.

Accordingly, a suitable adjuvant for use in the disclosed compositions is AS01, a liposome based adjuvant containing MPL and QS-21. The liposomes, which are the vehicles for the MPL and QS-21 immuno-enhancers, are composed of dioleoyl phosphatidylcholine (DOPC) and cholesterol in a phosphate buffered saline solution. AS01_{B-4} is a particularly preferred variant of the AS01 adjuvant, composed of immuno-enhancers QS-21 (a triterpene glycoside purified from the bark of Quillaja saponaria) and MPL (3-D Monophosphoryl lipid A), with DOPC/cholesterol liposomes, as vehicles for these immuno-enhancers, and sorbitol in a PBS solution. In particular, a single human dose of AS01_{B-4} (0.5 mL) contains 50 μg of QS-21 and 50 μg of MPL. AS01_{E-4} corresponds to a two-fold dilution of AS01_{B-4}. i.e. it contains 25 μg of QS-21 and 25 μg of MPL per human dose.

In one embodiment, there is provided an immunogenic combination for use in a method of treating chronic hepatitis B infection (CHB) and/or chronic hepatitis D infection (CHD) in a human, the immunogenic combination comprising a composition comprising a recombinant hepatitis B surface antigen (HBs), a recombinant hepatitis B virus core antigen (HBc) and an adjuvant. In one embodiment, the immunogenic combination comprises a composition comprising a recombinant hepatitis B surface antigen (HBs), a truncated recombinant hepatitis B virus core antigen (HBc) and an adjuvant. In one embodiment, the immunogenic combination comprises a composition comprising a recombinant HBs, a truncated recombinant HBc and an AS01 adjuvant. In a particular embodiment the immunogenic combination comprises a composition comprising a truncated recombinant HBc and a recombinant HBs in a ratio of 4:1 or more, and an AS01 adjuvant, for example AS01_{B-4} or AS01_{E-4}.

In one embodiment, there is provided an immunogenic combination for use in a method of treating chronic hepatitis B infection (CHB) and/or chronic hepatitis D infection (CHD) in a human, the immunogenic combination comprising:
a) a composition comprising an antisense oligonucleotide 10 to 30 nucleosides in length, targeted to a HBV nucleic acid (an HBV ASO);
b) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) a composition comprising a recombinant hepatitis B surface antigen (HBs), a recombinant hepatitis B virus core antigen (HBc) and an adjuvant,
wherein the method comprises administering the compositions sequentially or concomitantly to the human.

In a particular embodiment, the HBV ASO administered in step a) of the method has 85-95% identity to the sequence GCAGAGGTGAAGCGAAGTGC (SEQ ID NO: 226 of WO2012/145697) complementary to the HBV genome sequence SEQ ID NO: 16 at nucleobases 1583-1602. In a particular embodiment, the HBV ASO administered in step a) of the method has the sequence GCAGAGGTGAAGCGAAGTGC (SEQ ID NO: 226 of WO2012/145697) complementary to the HBV genome sequence SEQ ID NO: 16 at nucleobases 1583-1602.

In another aspect, there is provided an immunogenic composition for use in a method of treating chronic hepatitis B infection (CHB) and/or chronic hepatitis D infection (CHD) in a human, the immunogenic composition comprising a replication-defective chimpanzee adenoviral (ChAd) vector comprising a polynucleotide encoding a hepatitis B surface antigen (HBs), a nucleic acid encoding a hepatitis B virus core antigen (HBc) and a nucleic acid encoding the human invariant chain (hIi) fused to the HBc, wherein the method comprises administration of the composition in a prime-boost regimen with at least one other immunogenic composition as provided herein. In one embodiment, the composition comprises a ChAd vector selected from the group consisting of ChAd3, ChAd63, ChAd83, ChAd155, ChAd157, Pan 5, Pan 6, Pan 7 (also referred to as C7) and Pan 9, in particular, ChAd63 or ChAd155. In certain embodiments the ChAd vector includes a vector insert encoding HBc and HBs, separated by a spacer which incorporates a sequence encoding the 2A cleaving region of the foot and mouth disease virus. In a particular embodiment, the composition comprises a ChAd155 vector which comprises a polynucleotide vector insert encoding hIi, HBc, 2A and HBs, for example, an insert encoding a construct having the structure shown in FIG. 12. In one embodiment, the composition comprises a ChAd155 vector which comprises a polynucleotide vector insert encoding the amino acid sequence of SEQ ID NO:9. In another embodiment, the composition comprises a ChAd155 vector which comprises a polynucleotide vector insert encoding the amino acid sequence of SEQ ID NO:15. In one embodiment, the composition comprises a ChAd155 vector which comprises a polynucleotide vector insert having the nucleotide sequence given in SEQ ID NO:10. In another embodiment, the composition comprises a ChAd155 vector which comprises a polynucleotide vector insert having the nucleotide sequence given in SEQ ID NO:14.

In a further aspect, there is provided an immunogenic composition for use in a method of treating chronic hepatitis B infection (CHB) and/or chronic hepatitis D infection (CHD) in a human, the immunogenic composition comprising a Modified Vaccinia Virus Ankara (MVA) vector comprising a polynucleotide encoding a hepatitis B surface antigen (HBs) and a nucleic acid encoding a hepatitis B virus core antigen (HBc) wherein the method comprises administration of the composition in a prime-boost regimen with at least one other immunogenic composition as provided herein. In one embodiment, the composition comprises an MVA vector which includes a vector insert encoding HBc and HBs, separated by a spacer which incorporates a sequence encoding the 2A cleavage region of the foot and mouth disease virus. In a particular embodiment, the composition comprises an MVA vector which comprises a polynucleotide vector insert encoding HBc, 2A and HBs, for example, an insert encoding a construct having the structure shown in FIG. 12. In one embodiment, the composition comprises an MVA vector which comprises a polynucleotide vector insert encoding the amino acid sequence of SEQ ID NO:5. In one embodiment, the composition comprises an MVA vector which comprises a polynucleotide vector insert having the nucleotide sequence given in SEQ ID NO:6.

In a further aspect, there is provided an immunogenic composition for use in a method of treating chronic hepatitis B infection (CHB) and/or chronic hepatitis D infection (CHD) in a human, the immunogenic composition comprising a recombinant hepatitis B surface antigen (HBs), a C-terminal truncated recombinant hepatitis B virus core antigen (HBc) and an adjuvant containing MPL and QS-21, wherein the method comprises administration of the composition in a prime-boost regimen with at least one other immunogenic composition as provided herein. In one embodiment, the composition comprises truncated recombinant HBc comprising the assembly domain of HBc, for example amino acids 1-149 of HBc. In one embodiment, the composition comprises a full length recombinant HBs, amino acids 1-149 of HBc and an adjuvant comprising MPL and QS-21. More specifically, a composition for use in a method of treating chronic hepatitis B infection (CHB) and/or chronic hepatitis D infection (CHD) in a human comprises a full length recombinant HBs (e.g. SEQ ID NO:1), amino acids 1-149 of HBc (e.g. SEQ ID NO:2) and an adjuvant comprising MPL and QS-21 and liposomes comprising dioleoyl phosphatidylcholine (DOPC) and cholesterol. In certain embodiments the recombinant protein HBs and HBc antigens are in the form of virus-like particles. In a particular embodiment the composition comprises a truncated recombinant HBc and a full length recombinant HBs in a ratio of 4:1 or more and an AS01 adjuvant. In certain embodiments, the composition comprises a truncated core antigen consisting of amino acids 1-149 of HBc (e.g. SEQ ID NO:2) and full length recombinant HBs (e.g. SEQ ID NO:1), in a 4:1 ratio and AS01_{B-4}.

In a further aspect, there is provided a composition for use in a method of treating chronic hepatitis B infection (CHB) and/or chronic hepatitis D infection (CHD) in a human, the composition comprising an antisense oligonucleotide 10 to 30 nucleosides in length, targeted to a HBV nucleic acid (an HBV ASO) wherein the method comprises administration of the composition in a therapeutic regimen with at least one immunogenic composition as provided herein. In one embodiment, the composition comprises an antisense oligonucleotide having a nucleotide sequence selected from SEQ ID NOs: 83-310 of WO2012/145697. In particular embodiments, the antisense oligonucleotide targeted to a HBV nucleic acid (HBV ASO) has a nucleotide sequence selected from SEQ ID NOs: 224-227 of WO2012/145697. In a particular embodiment, the HBV ASO has the sequence GCAGAGGTGAAGCGAAGTGC (SEQ ID NO: 226 of WO2012/145697) complementary to the HBV genome sequence SEQ ID NO: 16 at nucleobases 1583-1602. In a particular embodiment, the HBV ASO is a modified oligonucleotide “gapmer” consisting of 20 linked nucleosides in which each internucleoside linkage is a phosphorothioate linkage and each cytosine is a 5-methylcytosine, having the sequence GCAGAGGTGAAGCGAAGTGC (SEQ ID NO: 226 of WO2012/145697) consisting of a 5′ wing segment consisting of five linked nucleosides GCAGA each comprising a 2′-O-methoxyethyl sugar, followed by ten linked deoxynucleosides GGTGAAGCGA and a 3′ wing segment consisting of five linked nucleosides AGTGC each comprising a 2′-O-methoxyethyl sugar.

In another aspect, there is provided an immunogenic combination comprising:
a) a composition comprising an antisense oligonucleotide (ASO) 10 to 30 nucleosides in length, targeted to a HBV nucleic acid (an HBV ASO)
b) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) a composition comprising a recombinant hepatitis B surface antigen (HBs), recombinant hepatitis B virus core antigen (HBc) and an adjuvant.

In a particular embodiment, the HBV ASO has 85-95% identity to the sequence GCAGAGGTGAAGCGAAGTGC (SEQ ID NO: 226 of WO2012/145697) complementary to the HBV genome sequence SEQ ID NO: 16 at nucleobases 1583-1602. In a particular embodiment, the HBV ASO has the sequence GCAGAGGTGAAGCGAAGTGC (SEQ ID NO: 226 of WO2012/145697) complementary to the HBV genome sequence SEQ ID NO: 16 at nucleobases 1583-1602.

The immunogenic combination may find use in a method for treating CHB and/or CHD in a human by administration of the compositions sequentially or concomitantly.

In one embodiment, part a) of the combination comprises a composition comprising an antisense oligonucleotide having a nucleotide sequence selected from SEQ ID NOs: 83-310 of WO2012/145697. In particular embodiments, the antisense oligonucleotide targeted to a HBV nucleic acid (HBV ASO) has a nucleotide sequence selected from SEQ ID NOs: 224-227 of WO2012/145697. In a particular embodiment, the HBV ASO has the sequence GCAGAGGTGAAGCGAAGTGC (SEQ ID NO: 226 of WO2012/145697) complementary to the HBV genome sequence SEQ ID NO: 16 at nucleobases 1583-1602. In a particular embodiment, the HBV ASO is a modified oligonucleotide “gapmer” consisting of 20 linked nucleosides in which each internucleoside linkage is a phosphorothioate linkage and each cytosine is a 5-methylcytosine, having the sequence GCAGAGGTGAAGCGAAGTGC (SEQ ID NO: 226 of WO2012/145697) consisting of a 5′ wing segment consisting of five linked nucleosides GCAGA each comprising a 2′-O-methoxyethyl sugar, followed by ten linked deoxynucleosides GGTGAAGCGA and a 3′ wing segment consisting of five linked nucleosides AGTGC each comprising a 2′-O-methoxyethyl sugar.

In one embodiment, part b) of the combination comprises a composition comprising a replication-defective chimpanzee adenoviral (ChAd) vector comprising a polynucleotide encoding a hepatitis B surface antigen (HBs), a nucleic acid encoding a hepatitis B virus core antigen (HBc) and a nucleic acid encoding the human invariant chain (hIi) fused to the HBc. In one embodiment, the composition comprises a ChAd vector selected from the group consisting of ChAd3, ChAd63, ChAd83, ChAd155, ChAd157, Pan 5, Pan 6, Pan 7 (also referred to as C7) and Pan 9, in particular, ChAd63 or ChAd155. In certain embodiments the ChAd vector includes a vector insert encoding HBc and HBs, separated by a spacer which incorporates a sequence encoding the 2A cleaving region of the foot and mouth disease virus. In a particular embodiment, the composition comprises a ChAd155 vector which comprises a polynucleotide vector insert encoding hIi, HBc, 2A and HBs, for example, an insert encoding a construct having the structure shown in FIG. 12. In one embodiment, the composition comprises a ChAd155 vector which comprises a polynucleotide vector insert encoding the amino acid sequence of SEQ ID NO:9. In another embodiment, the composition comprises a ChAd155 vector which comprises a polynucleotide vector insert encoding the amino acid sequence of SEQ ID NO:15. In one embodiment, the composition comprises a ChAd155 vector which comprises a polynucleotide vector insert having the nucleotide sequence given in SEQ ID NO:10. In another embodiment, the composition comprises a ChAd155 vector which comprises a polynucleotide vector insert having the nucleotide sequence given in SEQ ID NO:14.

In one embodiment, part c) of the combination comprises a composition comprising an MVA vector which includes a vector insert encoding HBc and HBs, separated by a spacer which incorporates a sequence encoding the 2A cleavage region of the foot and mouth disease virus. In a particular embodiment, the composition comprises an MVA vector which comprises a polynucleotide vector insert encoding HBc, 2A and HBs, for example, an insert encoding a construct having the structure shown in FIG. 12. In one embodiment, the composition comprises an MVA vector which comprises a polynucleotide vector insert encoding the amino acid sequence of SEQ ID NO:5. In one embodiment, the composition comprises an MVA vector which comprises a polynucleotide vector insert having the nucleotide sequence given in SEQ ID NO:6.

In one embodiment, part d) of the combination comprises a composition comprising a recombinant hepatitis B surface antigen (HBs), a C-terminal truncated recombinant hepatitis B virus core antigen (HBc) and an adjuvant containing MPL and QS-21. In one embodiment, the composition comprises truncated recombinant HBc comprising the assembly domain of HBc, for example amino acids 1-149 of HBc. In one embodiment, the composition comprises a full length recombinant HBs, amino acids 1-149 of HBc and an adjuvant comprising MPL and QS-21. More specifically, a composition for use in a method of treating chronic hepatitis B infection (CHB) and/or chronic hepatitis D infection (CHD) in a human comprises a full length recombinant HBs (e.g. SEQ ID NO:1), amino acids 1-149 of HBc (e.g. SEQ ID NO:2) and an adjuvant comprising MPL and QS-21 and liposomes comprising dioleoyl phosphatidylcholine (DOPC) and cholesterol. In certain embodiments the recombinant protein HBs and HBc antigens are in the form of virus-like particles. In a particular embodiment the composition comprises a truncated recombinant HBc and a full length recombinant HBs in a ratio of 4:1 or more and an AS01 adjuvant. In certain embodiments, the composition comprises a truncated core antigen consisting of amino acids 1-149 of HBc (e.g. SEQ ID NO:2) and full length recombinant HBs (e.g. SEQ ID NO:1), in a 4:1 ratio and AS01_{B-4}.

In another aspect, there is provided the use of an immunogenic composition in the manufacture of a medicament for treating chronic hepatitis B infection (CHB) and/or chronic hepatitis D infection (CHD) in a human, the immunogenic composition comprising a replication-defective chimpanzee adenoviral (ChAd) vector comprising a polynucleotide encoding a hepatitis B surface antigen (HBs), a nucleic acid encoding a hepatitis B virus core antigen (HBc) and a nucleic acid encoding the human invariant chain (hIi) fused to the HBc, wherein the method of treating chronic hepatitis B infection comprises administration of the composition in a prime-boost regimen with at least one other immunogenic composition as provided herein. In one embodiment, the composition comprises a ChAd vector selected from the group consisting of ChAd3, ChAd63, ChAd83, ChAd155, ChAd157, Pan 5, Pan 6, Pan 7 (also referred to as C7) and Pan 9, in particular, ChAd63 or ChAd155. In certain embodiments the ChAd vector includes a vector insert encoding HBc and HBs, separated by a spacer which incorporates a sequence encoding the 2A cleaving region of the foot and mouth disease virus. In a particular embodiment, the composition comprises a ChAd155 vector which comprises a polynucleotide vector insert encoding hIi, HBc, 2A and HBs, for example, an insert encoding a construct having the structure shown in FIG. 13. In certain embodiments, the vector insert encodes HBc (e.g. SEQ ID NO:11 or an amino acid sequence at least 98% homologous thereto) and HBs (e.g. SEQ ID NO:1 or an amino acid sequence at least 98% homologous thereto), separated by a sequence encoding a spacer which incorporates the 2A cleaving region of the foot and mouth disease virus (e.g. SEQ ID NO:3 or an amino acid sequence at least 98% homologous thereto). In certain embodiment, HBc (e.g. SEQ ID NO:11 or an amino acid sequence at least 98% homologous thereto) is fused to hIi (e.g. SEQ ID NO:7 or an amino acid sequence at least 98% homologous thereto or SEQ ID NO:12 or an amino acid sequence at least 98% homologous thereto). For example, HBc (e.g. SEQ ID NO:11) is fused to hIi (e.g. SEQ ID NO:7), or HBc (e.g. SEQ ID NO:11) is fused to hIi (e.g. SEQ ID NO:12). In one embodiment, the composition comprises a ChAd155 vector which comprises a polynucleotide vector insert encoding the amino acid sequence of SEQ ID NO:9. In an alternative embodiment, the composition comprises a ChAd155 vector which comprises a polynucleotide vector insert encoding the amino acid sequence of SEQ ID NO:15. In one embodiment, the composition comprises a ChAd155 vector which comprises a polynucleotide vector insert having the nucleotide sequence given in SEQ ID NO:10. In an alternative embodiment, the composition comprises a ChAd155 vector which comprises a polynucleotide vector insert having the nucleotide sequence given in SEQ ID NO:14.

In a further aspect, there is provided the use of an immunogenic composition in the manufacture of a medicament for treating chronic hepatitis B infection (CHB) and/or chronic hepatitis D infection (CHD) in a human, the immunogenic composition comprising a Modified Vaccinia Virus Ankara (MVA) vector comprising a polynucleotide encoding a hepatitis B surface antigen (HBs) and a nucleic acid encoding a hepatitis B virus core antigen (HBc) wherein the method of treating chronic hepatitis B infection comprises administration of the composition in a prime-boost regimen with at least one other immunogenic composition as provided herein. In one embodiment, the composition comprises an MVA vector which includes a vector insert encoding HBc and HBs, separated by a spacer which incorporates a sequence encoding the 2A cleavage region of the foot and mouth disease virus. In a particular embodiment, the composition comprises an MVA vector which comprises a polynucleotide vector insert encoding HBc, 2A and HBs, for example, an insert encoding a construct having the structure shown in FIG. 12. In certain embodiments, the vector insert encodes HBc (e.g. SEQ ID NO:11 or an amino acid sequence at least 98% homologous thereto) and HBs (e.g. SEQ ID NO:1 or an amino acid sequence at least 98% homologous thereto), separated by a sequence encoding a spacer which incorporates the 2A cleaving region of the foot and mouth disease virus (e.g. SEQ ID NO:3 or an amino acid sequence at least 98% homologous thereto). In one embodiment, the composition comprises an MVA vector which comprises a polynucleotide vector insert encoding the amino acid sequence of SEQ ID NO:5. In one embodiment, the composition comprises an MVA vector which comprises a polynucleotide vector insert having the nucleotide sequence given in SEQ ID NO:6.

In a further aspect, there is provided the use of an immunogenic composition in the manufacture of a medicament for treating chronic hepatitis B infection (CHB) and/or chronic hepatitis D infection (CHD) in a human, the immunogenic composition comprising a recombinant hepatitis B surface antigen (HBs), a C-terminal truncated recombinant hepatitis B virus core antigen (HBc) and an adjuvant containing MPL and QS-21, wherein the method of treating chronic hepatitis B infection comprises administration of the composition in a prime-boost regimen with at least one other immunogenic composition as provided herein. In one embodiment, the composition comprises truncated recombinant HBc comprising the assembly domain of HBc, for example amino acids 1-149 of HBc. In one embodiment, the composition comprises a full length recombinant HBs (e.g. SEQ ID NO:1), amino acids 1-149 of HBc (e.g. SEQ ID NO:2) and an adjuvant comprising MPL and QS-21 (e.g. an AS01 adjuvant, for example AS01_{B-4} or AS01_{E-4}). In certain embodiments the recombinant protein HBs and HBc antigens are in the form of virus-like particles.

In a further aspect, there is provided the use of an composition in the manufacture of a medicament for treating chronic hepatitis B infection (CHB) and/or chronic hepatitis D infection (CHD) in a human, the composition comprising an antisense oligonucleotide 10 to 30 nucleosides in length, targeted to a HBV nucleic acid (an HBV ASO). In one embodiment, the composition comprises an antisense oligonucleotide having a nucleotide sequence selected from SEQ ID NOs: 83-310 of WO2012/145697. In particular embodiments, the antisense oligonucleotide targeted to a HBV nucleic acid (HBV ASO) has a nucleotide sequence selected from SEQ ID NOs: 224-227 of WO2012/145697. In a particular embodiment, the HBV ASO has the sequence GCAGAGGTGAAGCGAAGTGC (SEQ ID NO: 226 of WO2012/145697) complementary to the HBV genome sequence SEQ ID NO: 16 at nucleobases 1583-1602. In a particular embodiment, the HBV ASO is a modified oligonucleotide “gapmer” consisting of 20 linked nucleosides in which each internucleoside linkage is a phosphorothioate linkage and each cytosine is a 5-methylcytosine, having the sequence GCAGAGGTGAAGCGAAGTGC (SEQ ID NO: 226 of WO2012/145697) consisting of a 5′ wing segment consisting of five linked nucleosides GCAGA each comprising a 2′-O-methoxyethyl sugar, followed by ten linked deoxynucleosides GGTGAAGCGA and a 3′ wing segment consisting of five linked nucleosides AGTGC each comprising a 2′-O-methoxyethyl sugar.

In one embodiment, there is provided the use of an immunogenic combination in the manufacture of a medicament for the treatment of chronic hepatitis B infection (CHB) and/or chronic hepatitis D infection (CHD) in a human, the immunogenic combination comprising:
i. a composition comprising an antisense oligonucleotide 10 to 30 nucleosides in length, targeted to a HBV nucleic acid (an HBV ASO);
ii. a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
iii. a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
iv. a composition comprising a recombinant hepatitis B surface antigen (HBs), recombinant hepatitis B virus core antigen (HBc) and an adjuvant,
wherein the method of treating chronic hepatitis B infection comprises administering the compositions sequentially or concomitantly to the human.

In a particular embodiment, the HBV ASO has 85-95% identity to the sequence GCAGAGGTGAAGCGAAGTGC (SEQ ID NO: 226 of WO2012/145697) complementary to the HBV genome sequence SEQ ID NO: 16 at nucleobases 1583-1602. In a particular embodiment, the HBV ASO has the sequence GCAGAGGTGAAGCGAAGTGC (SEQ ID NO: 226 of WO2012/145697) complementary to the HBV genome sequence SEQ ID NO: 16 at nucleobases 1583-1602.

In a particular embodiment, the use of an immunogenic combination in the manufacture of a medicament for the treatment of CHB and/or CHD comprises:
i. a composition comprising an antisense oligonucleotide 10 to 30 nucleosides in length, targeted to a HBV nucleic acid (an HBV ASO);
ii. a composition comprising a replication-defective ChAd vector comprising a polynucleotide encoding a HBs, a nucleic acid encoding a HBc and a polynucleotide encoding a hIi;
iii. a composition comprising an MVA vector comprising a polynucleotide encoding a HBs and a nucleic acid encoding a HBc; and
iv. a composition comprising a recombinant HBs, a truncated HBc and an adjuvant comprising MPL and QS-21,
wherein the method of treating CHB and/or CHD comprises the steps of:
   a) administering composition i. to the human;
   b) administering composition ii. to the human;
   c) administering composition iii. to the human; and
   d) administering composition iv. to the human,
   wherein the steps of the method are carried out sequentially, with step a) preceding step b), step b) preceding step c) and step c) preceding step d). In a further embodiment, step a) is repeated. In a further embodiment, step d) is repeated and the steps of the method are carried out sequentially in the order a), b), c), c), d). In another embodiment, step d) is carried out concomitantly with step b) and/or with step b).

In a particular embodiment, the HBV ASO has 85-95% identity to the sequence GCAGAGGTGAAGCGAAGTGC (SEQ ID NO: 226 of WO2012/145697) complementary to the HBV genome sequence SEQ ID NO: 16 at nucleobases 1583-1602. In a particular embodiment, the HBV ASO has the sequence GCAGAGGTGAAGCGAAGTGC (SEQ ID NO: 226 of WO2012/145697) complementary to the HBV genome sequence SEQ ID NO: 16 at nucleobases 1583-1602

In a further aspect, the present invention provides a kit comprising:
a) a composition comprising an antisense oligonucleotide 10 to 30 nucleosides in length, targeted to a HBV nucleic acid (an HBV ASO);
b) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) a composition comprising a recombinant hepatitis B surface antigen (HBs), recombinant hepatitis B virus core antigen (HBc) and an adjuvant,
with instructions for administration of the components sequentially or concomitantly for the treatment of CHB and/or CHD.

In a particular embodiment, the HBV ASO has 85-95% identity to the sequence GCAGAGGTGAAGCGAAGTGC (SEQ ID NO: 226 of WO2012/145697) complementary to the HBV genome sequence SEQ ID NO: 16 at nucleobases 1583-1602. In a particular embodiment, the HBV ASO has the sequence GCAGAGGTGAAGCGAAGTGC (SEQ ID NO: 226 of WO2012/145697) complementary to the HBV genome sequence SEQ ID NO: 16 at nucleobases 1583-1602

### Administration

In one embodiment of the disclosed methods, the disclosed compositions are administered via intranasal, intramuscular, subcutaneous, intradermal, or topical routes. Preferably, administration is via an intramuscular route.

An intranasal administration is the administration of the composition to the mucosa of the complete respiratory tract including the lung. More particularly, the composition is administered to the mucosa of the nose. In one embodiment, an intranasal administration is achieved by means of spray or aerosol. Intramuscular administration refers to the injection of a composition into any muscle of an individual. Exemplary intramuscular injections are administered into the deltoid, vastus lateralis or the ventrogluteal and dorsogluteal areas. Preferably, administration is into the deltoid. Subcutaneous administration refers to the injection of the composition into the hypodermis. Intradermal administration refers to the injection of a composition into the dermis between the layers of the skin. Topical administration is the administration of the composition to any part of the skin or mucosa without penetrating the skin with a needle or a comparable device. The composition may be administered topically to the mucosa of the mouth, nose, genital region and/or rectum. Topical administration includes administration means such as sublingual and/or buccal administration. Sublingual administration is the administration of the composition under the tongue (for example, using an oral thin film (OTF)). Buccal administration is the administration of the vector via the buccal mucosa of the cheek.

The methods disclosed herein can take the form of a prime-boost immunisation regimen. Accordingly, herein disclosed are compositions for use in a method of treatment of CHB and/or CHD which is a prime-boost immunisation method. In many cases, a single administration of an immunogenic composition is not sufficient to generate the number of long-lasting immune cells which is required for effective protection or for therapeutically treating a disease. Consequently, repeated challenge with a biological preparation specific for a specific pathogen or disease may be required in order to establish lasting and protective immunity against said pathogen or disease or to treat or functionally cure a given disease. An administration regimen comprising the repeated administration of an immunogenic composition or vaccine directed against the same pathogen or disease is referred to as a “prime-boost regimen”. In one embodiment, a prime-boost regimen involves at least two administrations of an immunogenic composition directed against hepatitis B. The first administration of the immunogenic composition is referred to as “priming” and any subsequent administration of the same immunogenic composition, or an immunogenic composition directed against the same pathogen, is referred to as “boosting”. It is to be understood that 2, 3, 4 or even 5 administrations for boosting the immune response are also contemplated. The period of time between prime and boost is, optionally, 1 week, 2 weeks, 4 weeks, 6 weeks 8 weeks or 12 weeks. More particularly, it is 4 weeks or 8 weeks. If more than one boost is performed, the subsequent boost is administered 1 week, 2 weeks, 4 weeks, 6 weeks, 8 weeks or 12 weeks, 6 months or 12 months after the preceding boost. For example, the interval between any two boosts may be 4 weeks or 8 weeks.

The compositions for use in the disclosed methods are administered in a therapeutic regimen which involves administration of a further immunogenic component, each formulated in different compositions. The compositions are favourably administered co-locationally at or near the same site. For example, the components can be administered intramuscularly, to the same side or extremity (“co-lateral” administration) or to opposite sides or extremities (“contra-lateral” administration). For example, in contra-lateral administration, a first composition may be administered to the left deltoid muscle and a second composition may be administered, sequentially or concomitantly, to the right deltoid muscle. Alternatively, in co-lateral administration, a first composition may be administered to the left deltoid muscle and a second composition may be administered, sequentially or concomitantly, also to the left deltoid muscle.

### General Manufacturing Processes

ChAd155-hIi-HBV:

The DNA fragment inserted as the transgene in the recombinant replication-defective simian (chimpanzee-derived) adenovirus group C vector ChAd155 is derived from two HBV protein antigens, the core nucleocapsid protein antigen HBc and the small surface antigen HBs, separated by the self-cleaving 2A region of the foot-and-mouth disease virus (FMDV) [Donnelly et al. 2001]. The 2A region of FMDV allows processing of the HBc-HBs fusion into separate protein antigens. In addition, the N-terminal part of the gene encoding the HBc protein has been fused to the gene encoding the human Major Histocompatibility Complex (MHC) class II-associated invariant chain p35 isoform (hIi). A schematic representation of the hIi-HBV transgene sequence is provided in (FIG. 13).

The 2A region (18 amino acids) has been supplemented with a spacer of 6 amino acids at its N-terminus; spacers of this nature have been reported to increase the efficiency of 2A mediated cleavage. The region 2A-mediated protease cleavage occurs at the C-terminus of 2A just ahead of the last proline in the 2A amino add sequence. The proline remains at the N-terminus of the HBs protein, while the 23 amino adds preceding the proline cleavage site remain with the hIi-HBc-2A polypeptide.

The expression of the transgene thereby results, following protease processing, in the production of two separate polypeptides: hIi-HBc-spacer-2A and HBs. For brevity the hIi-HBc-spacer-2A polypeptide is referred to as the hIi-HBc protein. When expressed in cell culture, the hIi-HBc antigen is detected in the cell culture supernatant whilst the HBs protein is detected in the intracellular fraction.

The expression cassette encoding the antigenic proteins, operatively linked to regulatory components in a manner which permits expression in a host cell, is assembled into the ChAd155 vector plasmid construct as previously described (see WO2016/198621 which is incorporated by reference for the purpose of disclosing ChAd155 vector sequences and methods) to give ChAd155-hIi-HBV. The hIi-HBV transgene is under the transcriptional control of human cytomegalovirus (hCMV) promoter and bovine growth hormone poly-adenylation signal (BGH pA). The expression cassette encodes the HBs, HBc and hIi amino acid sequences, in which the hIi sequence is fused to the HBc N-terminal of HBc and the HBs and HBc sequences are separated by a spacer which incorporates a 2A cleaving region of the foot and mouth disease virus, for processing of the HBc and HBs into separate proteins.

To generate recombinant ChAd155 adenoviruses which are replication deficient, the function of the deleted gene region required for replication and infectivity of the adenovirus must be supplied to the recombinant virus by a helper virus or cell line, i.e., a complementation or packaging cell line. A particularly suitable complementation cell line is the Procell92 cell line. The Procell92 cell line is based on HEK 293 cells which express adenoviral E1 genes, transfected with the Tet repressor under control of the human phosphoglycerate kinase-1 (PGK) promoter, and the G418-resistance gene (Vitelli et al. PLOS One (2013) 8(e55435):1-9). Procell92.S is adapted for growth in suspension conditions and is useful for producing adenoviral vectors expressing toxic proteins.

### Production of the ChAd155-hIi-HBV Drug Substance:

The manufacturing of the ChAd155-hIi-HBV viral particles (Drug Substance) involves culture of Procell-92.S cells at 5e5 cell/ml cell density at infection. The cells are then infected with ChAd155-hIi-HBV Master Viral Seed (MVS) using a multiplicity of infection of 200 vp/cell. The ChAd155-hIi-HBV virus harvest is purified following cell lysis, lysate clarification and concentration (filtration steps) by a multi-step process which includes anion exchange chromatography.

### Vaccine Formulation and Filling

The purified ChAd155-hIi-HBV bulk Drug Substance is subsequently processed as follows:
Dilution of the purified ChAd155-hIi-HBV Drug Substance in the formulation buffer.
Sterile filtration.
Filling of the final containers.

The ChAd155-hIi-HBV vaccine is a liquid formulation contained in vials. The formulation buffer includes Tris (10 mM), L-Histidine (10 mM), NaCl (75 mM), MgCl (1 mM) and EDTA (0.1 mM) with sucrose (5% w/v), polysorbate-80 (0.02% w/v) and ethanol (0.5% w/v), adjusted to pH 7.4 with HCl (water for injection to final volume).

MVA-HBV:

MVA-HBV is a recombinant modified vaccinia virus Ankara (MVA) carrying two different proteins of HBV: Core and S proteins, separated by 2A peptide. The MVA-HBV construct was generated from the MVA-Red vector system [Di Lullo et al. 2010], derived from the MVA virus seed batch from attenuation passage 571 (termed MVA-571) that was described by Professor Anton Mayr [Mayr, A. et al. 1978].

The MVA-HBV transgene encodes the core nucleocapsid protein HBc and the small surface antigen HBs of HBV. The HBc-HBs sequence is separated by the self-cleaving 2A region of the foot-and-mouth disease virus that allows processing of the fusion protein into separate HBc and HBs antigens as described above for the adenoviral vector. A schematic representation of the transgene is provided in FIG. 12.

The expression of the transgene, following protease processing, results in the production of two separate polypeptides: HBc-spacer-2A and HBs. For brevity the HBc-spacer-2A polypeptide is referred to as the HBc protein.

The expression cassette was subcloned into the MVA shuttle vector p94-elisaRen generating the transfer vector p94-HBV. p94-HBV contains the antigen expression cassette under the vaccinia P7.5 early/late promoter control and flanked by FlankIII-2 region and FlankIII-1 regions to allow insertion in the del III of MVA by homologous recombination.

The production of the recombinant virus was based on two events of in vivo recombination in CEF cells

Briefly, primary chick embryo fibroblasts (CEF) were infected with MVA-Red and then transfected with p94-HBV carrying the antigen transgene (as well as the EGFP marker gene under control of the synthetic promoter sP). The first recombination event occurs between homologous sequences (FlankIII-1 and -2 regions) present in both the MVA-Red genome and the transfer vector p94-HBV and results in replacement of the Hcred protein gene with transgene/eGFP cassette. Infected cells containing MVA-Green intermediate are isolated by FACS sorting and used to infect fresh CEF. The intermediate recombinant MVA, resulting from first recombination, carries both the transgene and the eGFP cassette but is instable due to the presence of repeated Z regions.

Thus, a spontaneous second recombination event involving Z regions occurs and removes the eGFP cassette. The resulting recombinant MVA is colourless and carries the transgene cassette.

Finally, markerless recombinant virus (MVA-HBV) infected cells were sorted by FACS, MVA-HBV was cloned by terminal dilution, and expanded in CEF by conventional methods.

### Production of the MVA-HBV Drug Substance

The MVA-HBV viral particles (Drug Substance) is manufactured in primary cell cultures of chicken embryo fibroblast (CEF) cells to a cell density between 1E6 and 2E6 cell/ml, and then infected with MVA-HBV Master Viral Seed (MVS) at a multiplicity of infection between 0.01 and 0.05 PFU/cell. The MVA-HBV virus harvest is purified by a multi-step process based on pelleting by centrifugation, resuspension and fractional gradient centrifugation steps.

### Vaccine Formulation and Filling

The purified MVA-HBV bulk Drug Substance is subsequently processed as follows:
Dilution of the purified MVA-HBV DS in the formulation buffer.
Filling of the final containers.

The MVA-HBV vaccine is a liquid formulation contained in vials. The formulation buffer includes Tris (hydroxymethyl) amino methane pH 7.7 (10 mM), NaCl (140 mM), and water for injection to final volume.

HBs-HBc Recombinant Protein Mix:

### Production of HBc Drug Substance

The HBc recombinant protein (Drug Substance) manufacturing process consists of inoculating a pre-culture flask using the recombinant E. coli working seed, followed by a fermentation process and a multi-step purification process including harvesting, extraction, clarification and multiple chromatography and filtration steps.

### Production of the HBs Drug Substance

The HBs recombinant protein (Drug Substance) manufacturing process consists of inoculating a pre-culture flask using the recombinant S. cerevisiae working seed, followed by a fermentation process and a multi-step purification process including harvesting, extraction, clarification and multiple chromatography and filtration steps.

### Vaccine Formulation and Filling

The purified HBs Drug Substance and HBc Drug Substance are diluted in the formulation buffer including sucrose as cryoprotectant and poloxamer as surfactant, filled and lyophilized in 4 mL clear glass vial.

While certain compounds, compositions, regimens and methods described herein have been described with specificity in accordance with certain embodiments, the following examples serve only to illustrate the compounds, compositions, regimens and methods described herein and are not intended to limit the same. Each of the references recited in the present application is incorporated herein by reference in its entirety.

### EXAMPLES

### Objectives of the Non-Clinical Experiments:

Strong and functional CD8⁺ and CD4⁺ T cell responses, particularly to the HBcAg, have been associated with HBV clearance and resolving infection [Boni, 2012; Li, 2011; Liang, 2011; Lau, 2002; Bertoletti, 2012]. Furthermore, anti-S antibodies prevent HBV spread to non-infected hepatocytes and may be key to control post-treatment rebound of HBV replication [Rehermann 2005; Neumann 2010]. The proposed vaccination regimen includes a heterologous prime-boost schedule with two viral vectored vaccines (ChAd155-hIi-HBV and MVA-HBV) coding for the hepatitis B core (HBc) and the hepatitis B surface (HBs) antigens in order to induce a strong CD8⁺ T-cell response, together with sequential or concomitant administration of AS01_{B-4}-adjuvanted HBc-HBs proteins in order to induce strong antigen-specific CD4⁺ T-cell and antibody responses in CHB patients. This vaccine-induced immune response, should ultimately translate to a substantial decrease in HBsAg concentration or HBsAg loss (i.e. HBsAg concentration below detectable level) considered as a marker for complete and durable control of HBV infection. Antisense therapy can directly target the mRNA transcripts for the HBV antigens, modulating expression of HBV mRNA and protein, and thereby reduce serum HBeAg and HBsAg levels. One objective of the non-clinical experiments is to assess the combination of HBV ASO with vaccine regimens in overcoming tolerance to HBs (anti-HBs Ab titres), inducing T cell responses and reducing circulating HBs antigen and HBV DNA levels.

### Materials and Methods for Examples Involving Antisense Oligonucleotides

### RNA Isolation

RNA analysis can be performed on total cellular RNA or poly(A)+ mRNA. Methods of RNA isolation are well known in the art, RNA is prepared using methods well known in the art, for example, using the TRIZOL Reagent (Invitrogen, Carlsbad, Calif.) according to the manufacturer's recommended protocols.

### Analysis of Inhibition of Target Levels or Expression

Inhibition of levels or expression of a HBV nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents may be obtained from Invitrogen (Carlsbad, Calif.). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc, Carlsbad, Calif.). Cyclophilin A expression is quantified by real time PCR, by being run simultaneously with the target, multiplexing, or separately. Total RNA is quantified using RIBOGREEN RNA quantification reagent (Invitrogen, Inc. Eugene, Oreg.). Methods of RNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a HBV nucleic acid. Methods for designing real-time PCR probes and primers are well known in the art, and may include the use of software such as PRIMER EXPRESS Software (Applied Biosystems, Foster City, Calif.).

### Quantitative Real-Time PCR Analysis of Target DNA Levels

Quantitation of target DNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Gene (or DNA) target quantities obtained by real time PCR are normalized using either the expression level of a gene whose expression is constant, such as cyclophilin A, or by quantifying total DNA using RIBOGREEN (Invitrogen, Inc. Carlsbad, Calif.). Cyclophilin A expression is quantified by real time PCR, by being run simultaneously with the target, multiplexing, or separately. Total DNA is quantified using RIBOGREEN RNA quantification reagent (Invitrogen, Inc. Eugene, Oreg.). Methods of DNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a HBV nucleic add. Methods for designing real-time PCR probes and primers are well known in the art, and may include the use of software such as PRIMER EXPRESS Software (Applied Biosystems, Foster City, Calif.).

### Example 1

### Antisense Inhibition of HBV Viral mRNA in HepG2.2.15 Cells by MOE Gapmers

The HepG2.2.15 cell is a widely used cell model for studying hepatitis B virus in vitro. In these cells, the HBV genome is integrated into several sites in the cellular DNA. The cells were originally derived from the human hepatoblastoma cell line HepG2 and are characterized by having stable HBV expression and replication in the culture system.

Antisense oligonucleotides were designed targeting a HBV viral nucleic acid and were tested for their effects on HBV mRNA in vitro. Cultured HepG2.2.15 cells at a density of 25,000 cells per well were transfected using electroporation with 15,000 nM antisense oligonucleotide. After a treatment period of approximately 24 hours, RNA was isolated from the cells and HBV mRNA levels were measured by quantitative real-time PCR. Viral primer probe set RTS3370 (forward sequence CTTGGTCATGGGCCATCAG, designated herein as SEQ ID NO: 17; reverse sequence CGGCTAGGAGTTCCGCAGTA, designated herein as SEQ ID NO: 18; probe sequence TGCGTGGAACCTTTTCGGCTCC, designated herein as SEQ ID NO: 19) was used to measure mRNA levels. RTS3370 detects the full length mRNA and the second portions of the pre-S1, pre-S2 and pre-C mRNA transcripts. The gapmers were also probed with additional primer probe sets. Viral primer probe set RTS3371 (forward sequence CCAAACCTTCGGACGGAAA, designated herein as SEQ ID NO: 20; reverse sequence TGAGGCCCACTCCCATAGG, designated herein as SEQ ID NO: 21; probe sequence CCCATCATCCTGGGCTTTCGGAAAAT, designated herein as SEQ ID NO: 22) was used also to measure mRNA levels. RTS3371 detects the full length mRNA and the second portions of the pre-S1, pre-S2 and pre-C mRNA transcripts, similar to RTS3370, but at different regions. Viral primer probe set RTS3372 (forward sequence ATCCTATCAACACTTCCGGAAACT, designated herein as SEQ ID NO: 23; reverse sequence CGACGCGGCGATTGAG, designated herein as SEQ ID NO: 24; probe sequence AAGAACTCCCTCGCCTCGCAGACG, designated herein as SEQ ID NO: 25) was used to measure mRNA levels. RTS3372 detects the full length genomic sequence. Viral primer probe set RTS3373MGB (forward sequence CCGACCTTGAGGCATACTTCA, designated herein as SEQ ID NO: 26; reverse sequence AATTTATGCCTACAGCCTCCTAGTACA, designated herein as SEQ ID NO: 27; probe sequence TTAAAGACTGGGAGGAGTTG, designated herein as SEQ ID NO: 28) was used to measure mRNA levels. RTS3373MGB detects the full length mRNA and the second portions of the pre-S1, pre-S2, pre-C, and pre-X mRNA transcripts.

HBV mRNA levels were adjusted according to total RNA content, as measured by RIBOGREEN®. Results are presented as percent inhibition of HBV, relative to untreated control cells.

The chimeric antisense oligonucleotides in Table 1 were designed as either 5-10-5 MOE gapmers, 3-10-3 MOE gapmers, or 2-10-2 MOE gapmers. The 5-10-5 MOE gapmers are 20 nucleosides in length, wherein the central gap segment comprises of ten 2′-deoxynucleosides and is flanked on both sides (in the 5′ and 3′ directions) by wings comprising five nucleosides each. The 3-10-3 MOE gapmers are 16 nucleosides in length, wherein the central gap segment comprises of ten 2′-deoxynucleosides and is flanked on both sides (in the 5′ and 3′ directions) by wings comprising three nucleosides each. The 2-10-2 MOE gapmers are 14 nucleosides in length, wherein the central gap segment comprises of ten 2′-deoxynucleosides and is flanked on both sides (in the 5′ and 3′ directions) by wings comprising two nucleosides each. Each nucleoside in the 5′ wing segment and each nucleoside in the 3′ wing segment has an MOE sugar modification. Each nucleoside in the central gap segment has a deoxy sugar modification. The internucleoside linkages throughout each gapmer are phosphorothioate (P═S) linkages. All cytosine residues throughout each gapmer are 5′-methylcytosines.

“Start site” indicates the 5′-most nucleotide to which the gapmer is targeted in the viral gene sequence. “Stop site” indicates the 3′-most nucleotide to which the gapmer is targeted viral gene sequence. Each gapmer listed in Table 1 is targeted to the viral genomic sequence, designated herein as SEQ ID NO: 16 (GENBANK Accession No. U95551.1).

TABLE 1
Inhibition of viral HBV mRNA levels by MOE gapmers targeted to SEQ ID NO: 16 (detected by
RTS3370, RTS3371, RTS3372, and RTS3373MGB) SQ ID NOs: 83-310 below correspond to SEQ ID
NOs: 83-310 of WO2012/145697

| | | | | | | | | SEQ |
|---|---|---|---|---|---|---|---|---|
| Start | Stop | | RTS3370 % | RTS3371 % | RTS3372 % | RTS3373MGB | | ID |
| Site | Site | Sequence | inhibition | inhibition | inhibition | % inhibition | Motif | NO |
| 58 | 77 | GAACTGGAGCCACCAGCAGG | 76 | 80 | 82 | 81 | 5-10-5 | 83 |
| 58 | 71 | GAGCCACCAGCAGG | 38 | 32 | 45 | 31 | 2-10-2 | 84 |
| 61 | 80 | CCTGAACTGGAGCCACCAGC | 68 | 71 | 67 | 66 | 5-10-5 | 85 |
| 62 | 77 | GAACTGGAGCCACCAG | 36 | 32 | 71 | 53 | 3-10-3 | 86 |
| 196 | 215 | AAAAACCCCGCCTGTAACAC | 69 | 74 | 80 | 88 | 5-10-5 | 87 |
| 199 | 218 | AAGAAAAACCCCGCCTGTAA | 60 | 60 | 64 | 64 | 5-10-5 | 88 |
| 205 | 224 | GTCAACAAGAAAAACCCCGC | 85 | 83 | 79 | 85 | 5-10-5 | 89 |
| 228 | 241 | GTATTGTGAGGATT | 28 | 18 | 0 | 16 | 2-10-2 | 90 |
| 229 | 242 | GGTATTGTGAGGAT | 40 | 37 | 19 | 34 | 2-10-2 | 91 |
| 244 | 263 | CACCACGAGTCTAGACTCTG | 74 | 73 | 62 | 75 | 5-10-5 | 92 |
| 245 | 260 | CACGAGTCTAGACTCT | 18 | 15 | 45 | 46 | 3-10-3 | 93 |
| 245 | 258 | CGAGTCTAGACTCT | 32 | 26 | 23 | 19 | 2-10-2 | 94 |
| 246 | 261 | CCACGAGTCTAGACTC | 34 | 35 | 63 | 60 | 3-10-3 | 95 |
| 247 | 266 | GTCCACCACGAGTCTAGACT | 75 | 77 | 64 | 75 | 5-10-5 | 96 |
| 250 | 269 | GAAGTCCACCACGAGTCTAG | 46 | 46 | 39 | 40 | 5-10-5 | 97 |
| 250 | 265 | TCCACCACGAGTCTAG | 38 | 39 | 65 | 59 | 3-10-3 | 98 |
| 251 | 270 | AGAAGTCCACCACGAGTCTA | 55 | 56 | 17 | 38 | 5-10-5 | 99 |
| 251 | 266 | GTCCACCACGAGTCTA | 34 | 35 | 64 | 51 | 3-10-3 | 100 |
| 252 | 271 | GAGAAGTCCACCACGAGTCT | 39 | 38 | 39 | 33 | 5-10-5 | 101 |
| 252 | 267 | AGTCCACCACGAGTCT | 47 | 51 | 50 | 45 | 3-10-3 | 102 |
| 253 | 272 | AGAGAAGTCCACCACGAGTC | 88 | 83 | 80 | 78 | 5-10-5 | 103 |
| 253 | 268 | AAGTCCACCACGAGTC | 46 | 50 | 56 | 46 | 3-10-3 | 104 |
| 254 | 273 | GAGAGAAGTCCACCACGAGT | 43 | 40 | 49 | 44 | 5-10-5 | 105 |
| 254 | 269 | GAAGTCCACCACGAGT | 41 | 46 | 51 | 44 | 3-10-3 | 106 |
| 254 | 267 | AGTCCACCACGAGT | 41 | 32 | 47 | 48 | 2-10-2 | 107 |
| 255 | 274 | TGAGAGAAGTCCACCACGAG | 50 | 57 | 55 | 55 | 5-10-5 | 108 |
| 255 | 270 | AGAAGTCCACCACGAG | 40 | 41 | 52 | 34 | 3-10-3 | 109 |
| 255 | 268 | AAGTCCACCACGAG | 26 | 29 | 19 | 23 | 2-10-2 | 110 |
| 256 | 275 | TTGAGAGAAGTCCACCACGA | 51 | 57 | 55 | 66 | 5-10-5 | 111 |
| 256 | 271 | GAGAAGTCCACCACGA | 30 | 31 | 43 | 33 | 3-10-3 | 112 |
| 256 | 269 | GAAGTCCACCACGA | 44 | 38 | 53 | 54 | 2-10-2 | 113 |
| 257 | 270 | AGAAGTCCACCACG | 39 | 42 | 32 | 25 | 2-10-2 | 114 |
| 258 | 273 | GAGAGAAGTCCACCAC | 54 | 52 | 60 | 48 | 3-10-3 | 115 |
| 258 | 271 | GAGAAGTCCACCAC | 29 | 30 | 25 | 19 | 2-10-2 | 116 |
| 259 | 274 | TGAGAGAAGTCCACCA | 39 | 44 | 47 | 38 | 3-10-3 | 117 |
| 259 | 272 | AGAGAAGTCCACCA | 31 | 29 | 3 | 15 | 2-10-2 | 118 |
| 260 | 273 | GAGAGAAGTCCACC | 21 | 19 | 23 | 18 | 2-10-2 | 119 |
| 261 | 274 | TGAGAGAAGTCCAC | 16 | 22 | 21 | 20 | 2-10-2 | 120 |
| 262 | 281 | AGAAAATTGAGAGAAGTCCA | 53 | 58 | 52 | 56 | 5-10-5 | 121 |
| 265 | 284 | CCTAGAAAATTGAGAGAAGT | 62 | 65 | 69 | 67 | 5-10-5 | 122 |
| 293 | 312 | ATTTTGGCCAAGACACACGG | 86 | 84 | 81 | 85 | 5-10-5 | 123 |
| 296 | 315 | CGAATTTTGGCCAAGACACA | 67 | 67 | 69 | 64 | 5-10-5 | 124 |
| 302 | 321 | GGACTGCGAATTTTGGCCAA | 77 | 75 | 73 | 76 | 5-10-5 | 125 |
| 360 | 379 | TCCAGCGATAACCAGGACAA | 89 | 90 | 77 | 91 | 5-10-5 | 126 |
| 366 | 385 | GACACATCCAGCGATAACCA | 83 | 85 | 75 | 86 | 5-10-5 | 127 |
| 369 | 388 | GCAGACACATCCAGCGATAA | 65 | 68 | 49 | 57 | 5-10-5 | 128 |
| 384 | 399 | GATAAAACGCCGCAGA | 37 | 46 | 53 | 35 | 3-10-3 | 129 |
| 384 | 397 | TAAAACGCCGCAGA | 36 | 36 | 33 | 33 | 2-10-2 | 130 |
| 385 | 398 | ATAAAACGCCGCAG | 12 | 7 | 19 | 15 | 2-10-2 | 131 |
| 386 | 401 | ATGATAAAACGCCGCA | 49 | 55 | 57 | 53 | 3-10-3 | 132 |
| 386 | 399 | GATAAAACGCCGCA | 39 | 39 | 45 | 37 | 2-10-2 | 133 |
| 387 | 400 | TGATAAAACGCCGC | 40 | 37 | 29 | 39 | 2-10-2 | 134 |
| 388 | 401 | ATGATAAAACGCCG | 22 | 24 | 9 | 22 | 2-10-2 | 135 |
| 411 | 430 | TGAGGCATAGCAGCAGGATG | 60 | 64 | 47 | 55 | 5-10-5 | 136 |
| 411 | 426 | GCATAGCAGCAGGATG | 62 | 64 | 71 | 60 | 3-10-3 | 137 |
| 411 | 424 | ATAGCAGCAGGATG | 44 | 34 | 30 | 48 | 2-10-2 | 138 |
| 412 | 431 | ATGAGGCATAGCAGCAGGAT | 45 | 54 | 71 | 62 | 5-10-5 | 139 |
| 412 | 427 | GGCATAGCAGCAGGAT | 72 | 75 | 80 | 71 | 3-10-3 | 140 |
| 412 | 425 | CATAGCAGCAGGAT | 29 | 24 | 24 | 20 | 2-10-2 | 141 |
| 413 | 432 | GATGAGGCATAGCAGCAGGA | 54 | 58 | 54 | 49 | 5-10-5 | 142 |
| 413 | 428 | AGGCATAGCAGCAGGA | 63 | 66 | 68 | 64 | 3-10-3 | 143 |
| 413 | 426 | GCATAGCAGCAGGA | 55 | 54 | 37 | 46 | 2-10-2 | 144 |
| 414 | 433 | AGATGAGGCATAGCAGCAGG | 85 | 87 | 74 | 82 | 5-10-5 | 20 |
| 414 | 429 | GAGGCATAGCAGCAGG | 64 | 64 | 80 | 68 | 3-10-3 | 145 |
| 414 | 427 | GGCATAGCAGCAGG | 58 | 54 | 41 | 45 | 2-10-2 | 146 |
| 415 | 430 | TGAGGCATAGCAGCAG | 59 | 59 | 66 | 64 | 3-10-3 | 147 |
| 415 | 428 | AGGCATAGCAGCAG | 58 | 55 | 38 | 41 | 2-10-2 | 148 |
| 416 | 431 | ATGAGGCATAGCAGCA | 56 | 54 | 65 | 56 | 3-10-3 | 149 |
| 416 | 429 | GAGGCATAGCAGCA | 64 | 62 | 64 | 57 | 2-10-2 | 150 |
| 417 | 432 | GATGAGGCATAGCAGC | 57 | 52 | 58 | 49 | 3-10-3 | 151 |
| 417 | 430 | TGAGGCATAGCAGC | 48 | 50 | 55 | 48 | 2-10-2 | 152 |
| 418 | 433 | AGATGAGGCATAGCAG | 50 | 52 | 64 | 51 | 3-10-3 | 153 |
| 418 | 431 | ATGAGGCATAGCAG | 36 | 31 | 36 | 26 | 2-10-2 | 154 |
| 419 | 434 | AAGATGAGGCATAGCA | 48 | 47 | 72 | 65 | 3-10-3 | 155 |
| 419 | 432 | GATGAGGCATAGCA | 44 | 28 | 0 | 14 | 2-10-2 | 156 |
| 420 | 435 | GAAGATGAGGCATAGC | 45 | 41 | 65 | 62 | 3-10-3 | 157 |
| 420 | 433 | AGATGAGGCATAGC | 41 | 43 | 37 | 29 | 2-10-2 | 158 |
| 421 | 436 | AGAAGATGAGGCATAG | 32 | 29 | 64 | 51 | 3-10-3 | 159 |
| 421 | 434 | AAGATGAGGCATAG | 21 | 18 | 26 | 27 | 2-10-2 | 160 |
| 422 | 437 | AAGAAGATGAGGCATA | 21 | 17 | 55 | 46 | 3-10-3 | 161 |
| 422 | 435 | GAAGATGAGGCATA | 25 | 24 | 23 | 25 | 2-10-2 | 162 |
| 423 | 436 | AGAAGATGAGGCAT | 21 | 17 | 25 | 19 | 2-10-2 | 163 |
| 424 | 437 | AAGAAGATGAGGCA | 17 | 11 | 38 | 27 | 2-10-2 | 164 |
| 454 | 473 | ACGGGCAACATACCTTGATA | 55 | 57 | 65 | 60 | 5-10-5 | 165 |
| 457 | 476 | CAAACGGGCAACATACCTTG | 73 | 77 | 77 | 74 | 5-10-5 | 166 |
| 457 | 472 | CGGGCAACATACCTTG | 60 | 61 | 73 | 70 | 3-10-3 | 167 |
| 458 | 473 | ACGGGCAACATACCTT | 58 | 63 | 64 | 58 | 3-10-3 | 168 |
| 458 | 471 | GGGCAACATACCTT | 58 | 56 | 57 | 46 | 2-10-2 | 169 |
| 459 | 472 | CGGGCAACATACCT | 49 | 43 | 47 | 37 | 2-10-2 | 170 |
| 460 | 473 | ACGGGCAACATACC | 50 | 50 | 54 | 51 | 2-10-2 | 171 |
| 463 | 482 | AGAGGACAAACGGGCAACAT | 64 | 68 | 64 | 71 | 5-10-5 | 172 |
| 466 | 485 | ATTAGAGGACAAACGGGCAA | 59 | 62 | 42 | 69 | 5-10-5 | 173 |
| 472 | 491 | CCTGGAATTAGAGGACAAAC | 78 | 81 | 73 | 86 | 5-10-5 | 174 |
| 475 | 494 | GATCCTGGAATTAGAGGACA | 56 | 65 | 61 | 72 | 5-10-5 | 175 |
| 639 | 654 | GGCCCACTCCCATAGG | 38 | 55 | 74 | 48 | 3-10-3 | 176 |
| 641 | 656 | GAGGCCCACTCCCATA | 30 | 46 | 77 | 54 | 3-10-3 | 177 |
| 642 | 657 | TGAGGCCCACTCCCAT | 58 | 57 | 84 | 66 | 3-10-3 | 178 |
| 643 | 658 | CTGAGGCCCACTCCCA | 38 | 53 | 70 | 66 | 3-10-3 | 179 |
| 670 | 689 | GGCACTAGTAAACTGAGCCA | 61 | 64 | 63 | 63 | 5-10-5 | 180 |
| 670 | 685 | CTAGTAAACTGAGCCA | 71 | 71 | 78 | 80 | 3-10-3 | 181 |
| 670 | 683 | AGTAAACTGAGCCA | 49 | 48 | 52 | 53 | 2-10-2 | 182 |
| 671 | 684 | TAGTAAACTGAGCC | 41 | 38 | 19 | 30 | 2-10-2 | 183 |
| 672 | 685 | CTAGTAAACTGAGC | 25 | 27 | 42 | 47 | 2-10-2 | 184 |
| 673 | 692 | AATGGCACTAGTAAACTGAG | 34 | 46 | 49 | 52 | 5-10-5 | 185 |
| 679 | 698 | TGAACAAATGGCACTAGTAA | 74 | 77 | 71 | 80 | 5-10-5 | 186 |
| 682 | 701 | CACTGAACAAATGGCACTAG | 82 | 83 | 71 | 82 | 5-10-5 | 187 |
| 687 | 702 | CCACTGAACAAATGGC | 72 | 73 | 76 | 80 | 3-10-3 | 188 |
| 688 | 707 | ACGAACCACTGAACAAATGG | 69 | 69 | 78 | 76 | 5-10-5 | 189 |
| 688 | 703 | ACCACTGAACAAATGG | 47 | 48 | 67 | 65 | 3-10-3 | 190 |
| 689 | 704 | AACCACTGAACAAATG | 33 | 33 | 39 | 41 | 3-10-3 | 191 |
| 690 | 705 | GAACCACTGAACAAAT | 50 | 49 | 63 | 48 | 3-10-3 | 192 |
| 691 | 710 | CCTACGAACCACTGAACAAA | 64 | 70 | 70 | 72 | 5-10-5 | 193 |
| 691 | 706 | CGAACCACTGAACAAA | 67 | 66 | 78 | 77 | 3-10-3 | 194 |
| 691 | 704 | AACCACTGAACAAA | 36 | 36 | 23 | 32 | 2-10-2 | 195 |
| 692 | 705 | GAACCACTGAACAA | 45 | 44 | 51 | 43 | 2-10-2 | 196 |
| 693 | 706 | CGAACCACTGAACA | 59 | 52 | 48 | 49 | 2-10-2 | 197 |
| 697 | 716 | GAAAGCCCTACGAACCACTG | 76 | 80 | 73 | 83 | 5-10-5 | 198 |
| 738 | 753 | CCACATCATCCATATA | 40 | 33 | 62 | 54 | 3-10-3 | 199 |
| 738 | 751 | ACATCATCCATATA | 19 | 9 | 30 | 27 | 2-10-2 | 200 |
| 739 | 754 | ACCACATCATCCATAT | 76 | 78 | 93 | 85 | 3-10-3 | 201 |
| 739 | 752 | CACATCATCCATAT | 45 | 35 | 24 | 17 | 2-10-2 | 202 |
| 740 | 753 | CCACATCATCCATA | 52 | 49 | 43 | 40 | 2-10-2 | 203 |
| 741 | 754 | ACCACATCATCCAT | 44 | 45 | 48 | 47 | 2-10-2 | 204 |
| 756 | 775 | TGTACAGACTTGGCCCCCAA | 47 | 56 | 55 | 68 | 5-10-5 | 205 |
| 823 | 842 | AGGGTTTAAATGTATACCCA | 66 | 71 | 64 | 72 | 5-10-5 | 206 |
| 1170 | 1189 | GCAAACACTTGGCACAGACC | 76 | 80 | 35 | 70 | 5-10-5 | 207 |
| 1176 | 1191 | CAGCAAACACTTGGCA | 42 | 44 | 56 | 54 | 3-10-3 | 208 |
| 1177 | 1192 | TCAGCAAACACTTGGC | 60 | 54 | 74 | 70 | 3-10-3 | 209 |
| 1259 | 1278 | CCGCAGTATGGATCGGCAGA | 88 | 82 | 57 | 80 | 5-10-5 | 210 |
| 1261 | 1276 | GCAGTATGGATCGGCA | 61 | 58 | 65 | 72 | 3-10-3 | 211 |
| 1262 | 1281 | GTTCCGCAGTATGGATCGGC | 84 | 81 | 71 | 83 | 5-10-5 | 212 |
| 1268 | 1287 | CTAGGAGTTCCGCAGTATGG | 78 | 68 | 70 | 79 | 5-10-5 | 213 |
| 1271 | 1290 | CGGCTAGGAGTTCCGCAGTA | 47 | 54 | 59 | 61 | 5-10-5 | 214 |
| 1277 | 1296 | AACAAGCGGCTAGGAGTTCC | 55 | 62 | 69 | 69 | 5-10-5 | 215 |
| 1280 | 1299 | CAAAACAAGCGGCTAGGAGT | 20 | 49 | 49 | 54 | 5-10-5 | 216 |
| 1283 | 1302 | GAGCAAAACAAGCGGCTAGG | 53 | 83 | 73 | 87 | 5-10-5 | 217 |
| 1286 | 1305 | TGCGAGCAAAACAAGCGGCT | 64 | 73 | 68 | 78 | 5-10-5 | 218 |
| 1413 | 1426 | ACAAAGGACGTCCC | 14 | 8 | 0 | 0 | 2-10-2 | 219 |
| 1515 | 1534 | GAGGTGCGCCCCGTGGTCGG | 68 | 81 | 61 | 80 | 5-10-5 | 220 |
| 1518 | 1537 | AGAGAGGTGCGCCCCGTGGT | 59 | 75 | 75 | 84 | 5-10-5 | 221 |
| 1521 | 1540 | TAAAGAGAGGTGCGCCCCGT | 63 | 76 | 83 | 78 | 5-10-5 | 222 |
| 1550 | 1563 | AAGGCACAGACGGG | 35 | 38 | 25 | 32 | 2-10-2 | 223 |
| 1577 | 1596 | GTGAAGCGAAGTGCACACGG | 88 | 91 | 84 | 93 | 5-10-5 | 224 |
| 1580 | 1599 | GAGGTGAAGCGAAGTGCACA | 70 | 75 | 71 | 82 | 5-10-5 | 225 |
| 1583 | 1602 | GCAGAGGTGAAGCGAAGTGC | 77 | 82 | 72 | 84 | 5-10-5 | 226 |
| 1586 | 1605 | CGTGCAGAGGTGAAGCGAAG | 72 | 73 | 67 | 80 | 5-10-5 | 227 |
| 1655 | 1674 | AGTCCAAGAGTCCTCTTATG | 66 | 68 | 54 | 68 | 5-10-5 | 228 |
| 1706 | 1719 | CAGTCTTTGAAGTA | 19 | 19 | 26 | 17 | 2-10-2 | 229 |
| 1778 | 1793 | TATGCCTACAGCCTCC | 64 | 60 | 64 | 63 | 3-10-3 | 230 |
| 1779 | 1794 | TTATGCCTACAGCCTC | 66 | 66 | 77 | 73 | 3-10-3 | 231 |
| 1780 | 1795 | TTTATGCCTACAGCCT | 56 | 55 | 68 | 67 | 3-10-3 | 232 |
| 1781 | 1796 | ATTTATGCCTACAGCC | 52 | 52 | 68 | 63 | 3-10-3 | 233 |
| 1782 | 1797 | AATTTATGCCTACAGC | 48 | 44 | 70 | 59 | 3-10-3 | 234 |
| 1783 | 1798 | CAATTTATGCCTACAG | 24 | 18 | 39 | 40 | 3-10-3 | 235 |
| 1784 | 1799 | CCAATTTATGCCTACA | 37 | 37 | 55 | 55 | 3-10-3 | 236 |
| 1785 | 1800 | ACCAATTTATGCCTAC | 35 | 36 | 60 | 55 | 3-10-3 | 237 |
| 1806 | 1825 | AAAGTTGCATGGTGCTGGTG | 42 | 55 | 75 | 61 | 5-10-5 | 238 |
| 1809 | 1828 | GAAAAAGTTGCATGGTGCTG | 45 | 56 | 64 | 53 | 5-10-5 | 239 |
| 1812 | 1831 | GGTGAAAAAGTTGCATGGTG | 71 | 70 | 80 | 72 | 5-10-5 | 240 |
| 1815 | 1834 | AGAGGTGAAAAAGTTGCATG | 51 | 57 | 77 | 82 | 5-10-5 | 241 |
| 1818 | 1837 | GGCAGAGGTGAAAAAGTTGC | 54 | 63 | 76 | 78 | 5-10-5 | 242 |
| 1821 | 1840 | TTAGGCAGAGGTGAAAAAGT | 61 | 65 | 80 | 66 | 5-10-5 | 243 |
| 1822 | 1837 | GGCAGAGGTGAAAAAG | 47 | 51 | 74 | 54 | 3-10-3 | 244 |
| 1823 | 1838 | AGGCAGAGGTGAAAAA | 47 | 40 | 76 | 54 | 3-10-3 | 245 |
| 1824 | 1843 | TGATTAGGCAGAGGTGAAAA | 41 | 39 | 62 | 29 | 5-10-5 | 246 |
| 1824 | 1839 | TAGGCAGAGGTGAAAA | 46 | 42 | 79 | 59 | 3-10-3 | 247 |
| 1826 | 1839 | TAGGCAGAGGTGAA | 40 | 33 | 44 | 31 | 2-10-2 | 248 |
| 1827 | 1846 | AGATGATTAGGCAGAGGTGA | 27 | 46 | 62 | 51 | 5-10-5 | 249 |
| 1861 | 1880 | AGCTTGGAGGCTTGAACAGT | 59 | 61 | 65 | 72 | 5-10-5 | 250 |
| 1864 | 1883 | CACAGCTTGGAGGCTTGAAC | 11 | 21 | 48 | 31 | 5-10-5 | 251 |
| 1865 | 1880 | AGCTTGGAGGCTTGAA | 13 | 1 | 45 | 40 | 3-10-3 | 252 |
| 1865 | 1878 | CTTGGAGGCTTGAA | 22 | 17 | 20 | 14 | 2-10-2 | 253 |
| 1866 | 1881 | CAGCTTGGAGGCTTGA | 29 | 19 | 51 | 45 | 3-10-3 | 254 |
| 1866 | 1879 | GCTTGGAGGCTTGA | 24 | 25 | 37 | 32 | 2-10-2 | 255 |
| 1867 | 1886 | AGGCACAGCTTGGAGGCTTG | 32 | 36 | 58 | 33 | 5-10-5 | 63 |
| 1867 | 1882 | ACAGCTTGGAGGCTTG | 1 | 4 | 23 | 12 | 3-10-3 | 256 |
| 1867 | 1880 | AGCTTGGAGGCTTG | 23 | 24 | 17 | 23 | 2-10-2 | 257 |
| 1868 | 1883 | CACAGCTTGGAGGCTT | 5 | 1 | 48 | 41 | 3-10-3 | 258 |
| 1868 | 1881 | CAGCTTGGAGGCTT | 21 | 20 | 0 | 18 | 2-10-2 | 259 |
| 1869 | 1884 | GCACAGCTTGGAGGCT | 14 | 10 | 50 | 37 | 3-10-3 | 260 |
| 1869 | 1882 | ACAGCTTGGAGGCT | 19 | 22 | 24 | 27 | 2-10-2 | 261 |
| 1870 | 1889 | CCAAGGCACAGCTTGGAGGC | 27 | 40 | 68 | 38 | 5-10-5 | 69 |
| 1870 | 1885 | GGCACAGCTTGGAGGC | 10 | 12 | 43 | 16 | 3-10-3 | 262 |
| 1870 | 1883 | CACAGCTTGGAGGC | 28 | 31 | 33 | 30 | 2-10-2 | 263 |
| 1871 | 1886 | AGGCACAGCTTGGAGG | 24 | 20 | 46 | 25 | 3-10-3 | 264 |
| 1871 | 1884 | GCACAGCTTGGAGG | 20 | 18 | 22 | 15 | 2-10-2 | 265 |
| 1872 | 1887 | AAGGCACAGCTTGGAG | 6 | 0 | 45 | 24 | 3-10-3 | 266 |
| 1872 | 1885 | GGCACAGCTTGGAG | 18 | 18 | 32 | 23 | 2-10-2 | 267 |
| 1873 | 1892 | CACCCAAGGCACAGCTTGGA | 18 | 8 | 55 | 16 | 5-10-5 | 268 |
| 1873 | 1888 | CAAGGCACAGCTTGGA | 9 | 0 | 31 | 15 | 3-10-3 | 269 |
| 1873 | 1886 | AGGCACAGCTTGGA | 23 | 9 | 27 | 10 | 2-10-2 | 270 |
| 1874 | 1889 | CCAAGGCACAGCTTGG | 0 | 0 | 39 | 25 | 3-10-3 | 271 |
| 1876 | 1895 | AGCCACCCAAGGCACAGCTT | 47 | 50 | 69 | 56 | 5-10-5 | 272 |
| 1879 | 1898 | CAAAGCCACCCAAGGCACAG | 27 | 27 | 55 | 30 | 5-10-5 | 273 |
| 1882 | 1901 | CCCCAAAGCCACCCAAGGCA | 34 | 40 | 54 | 39 | 5-10-5 | 274 |
| 1885 | 1904 | ATGCCCCAAAGCCACCCAAG | 41 | 43 | 54 | 52 | 5-10-5 | 275 |
| 1888 | 1907 | TCCATGCCCCAAAGCCACCC | 40 | 42 | 72 | 40 | 5-10-5 | 276 |
| 1891 | 1910 | ATGTCCATGCCCCAAAGCCA | 35 | 33 | 70 | 40 | 5-10-5 | 277 |
| 1918 | 1933 | CTCCAAATTCTTTATA | 9 | 2 | 53 | 41 | 3-10-3 | 278 |
| 1918 | 1931 | CCAAATTCTTTATA | 28 | 22 | 7 | 22 | 2-10-2 | 279 |
| 1919 | 1934 | GCTCCAAATTCTTTAT | 43 | 39 | 72 | 57 | 3-10-3 | 280 |
| 1919 | 1932 | TCCAAATTCTTTAT | 19 | 11 | 0 | 2 | 2-10-2 | 281 |
| 1920 | 1933 | CTCCAAATTCTTTA | 19 | 11 | 0 | 0 | 2-10-2 | 282 |
| 1921 | 1934 | GCTCCAAATTCTTT | 50 | 48 | 61 | 55 | 2-10-2 | 283 |
| 1957 | 1976 | GGAAAGAAGTCAGAAGGCAA | 17 | 14 | 81 | 39 | 5-10-5 | 284 |
| 2270 | 2285 | GTGCGAATCCACACTC | 21 | 4 | 36 | 11 | 3-10-3 | 285 |
| 2270 | 2283 | GCGAATCCACACTC | 32 | 29 | 41 | 33 | 2-10-2 | 286 |
| 2271 | 2284 | TGCGAATCCACACT | 28 | 20 | 25 | 11 | 2-10-2 | 287 |
| 2272 | 2285 | GTGCGAATCCACAC | 28 | 20 | 32 | 22 | 2-10-2 | 288 |
| 2368 | 2387 | GAGGGAGTTCTTCTTCTAGG | 24 | 22 | 90 | 48 | 5-10-5 | 289 |
| 2378 | 2393 | CGAGGCGAGGGAGTTC | 12 | 1 | 65 | 10 | 3-10-3 | 290 |
| 2378 | 2391 | AGGCGAGGGAGTTC | 17 | 18 | 29 | 25 | 2-10-2 | 291 |
| 2379 | 2394 | GCGAGGCGAGGGAGTT | 18 | 13 | 82 | 37 | 3-10-3 | 292 |
| 2379 | 2392 | GAGGCGAGGGAGTT | 29 | 22 | 54 | 30 | 2-10-2 | 293 |
| 2380 | 2395 | TGCGAGGCGAGGGAGT | 13 | 11 | 69 | 44 | 3-10-3 | 294 |
| 2380 | 2393 | CGAGGCGAGGGAGT | 25 | 20 | 53 | 42 | 2-10-2 | 295 |
| 2381 | 2396 | CTGCGAGGCGAGGGAG | 17 | 14 | 79 | 53 | 3-10-3 | 296 |
| 2381 | 2394 | GCGAGGCGAGGGAG | 33 | 29 | 66 | 48 | 2-10-2 | 297 |
| 2382 | 2397 | TCTGCGAGGCGAGGGA | 18 | 4 | 77 | 47 | 3-10-3 | 298 |
| 2420 | 2439 | CCGAGATTGAGATCTTCTGC | 12 | 18 | 83 | 28 | 5-10-5 | 299 |
| 2459 | 2478 | CCCACCTTATGAGTCCAAGG | 14 | 19 | 80 | 36 | 5-10-5 | 300 |
| 2819 | 2838 | TGTTCCCAAGAATATGGTGA | 29 | 32 | 78 | 44 | 5-10-5 | 301 |
| 2820 | 2835 | TCCCAAGAATATGGTG | 10 | 10 | 68 | 40 | 3-10-3 | 302 |
| 2821 | 2836 | TTCCCAAGAATATGGT | 5 | 0 | 62 | 24 | 3-10-3 | 303 |
| 2822 | 2837 | GTTCCCAAGAATATGG | 6 | 2 | 42 | 16 | 3-10-3 | 304 |
| 2823 | 2838 | TGTTCCCAAGAATATG | 18 | 18 | 47 | 18 | 3-10-3 | 305 |
| 2824 | 2839 | TTGTTCCCAAGAATAT | 7 | 5 | 57 | 19 | 3-10-3 | 306 |
| 2825 | 2838 | TGTTCCCAAGAATA | 25 | 20 | 44 | 25 | 2-10-2 | 307 |
| 2873 | 2892 | GAAAGAATCCCAGAGGATTG | 8 | 4 | 61 | 22 | 5-10-5 | 308 |
| 3161 | 3180 | ACTGCATGGCCTGAGGATGA | 47 | 46 | 82 | 54 | 5-10-5 | 309 |
| 3163 | 3182 | CCACTGCATGGCCTGAGGAT | 25 | 34 | 69 | 19 | 5-10-5 | 310 |

### Example 2

### Tolerability of MOE Gapmers Targeting HBV in BALB/c Mice

BALB/c mice (Charles River, Mass.) are a multipurpose model of mice, frequently utilized for safety and efficacy testing. The mice were treated with antisense oligonucleotides selected from Example 1 above and evaluated for changes in the levels of various metabolic markers.

Groups of four BALB/c mice each were injected subcutaneously twice a week for 3 weeks with 50 mg/kg of SEQ ID NO: 83, SEQ ID NO: 224, SEQ ID NO: 88, SEQ ID NO: 103, SEQ ID NO: 20, SEQ ID NO: 116, SEQ ID NO: 187, SEQ ID NO: 210, SEQ ID NO: 212, SEQ ID NO: 226, SEQ ID NO: 24, SEQ ID NO: 39, SEQ ID NO: 46, SEQ ID NO: 50, SEQ ID NO: 140, SEQ ID NO: 17, SEQ ID NO: 27, SEQ ID NO: 40 and SEQ ID NO: 74, all sequence numbers of WO2012/145697. A group of four BALB/c mice were injected subcutaneously twice a week for 3 weeks with 50 mg/kg of antisense oligonucleotide having the sequence CCTTCCCTGAAGGTTCCTCC (SEQ ID NO: 320 of WO2012/145697), a 5-10-5 MOE gapmer with no known homology to any human or mouse gene sequence. Another group of 4 BALB/c mice was injected subcutaneously twice a week for 3 weeks with PBS. This group of mice served as the control group. Three days after the last dose at each time point, body weights were taken, mice were euthanized and organs and plasma were harvested for further analysis.

### Body and Organ Weights

The body weights of the mice were measured pre-dose and at the end of each treatment period. The body weights are presented in Table 2, and are expressed as percent change from the weight taken before the start of treatment. Liver, spleen and kidney weights were measured at the end of the study, and are presented in Table 3 as a percentage difference from the respective organ weights of the PBS control. The results indicate that most of the ISIS oligonucleotides did not cause any adverse effects on body or organ weights.

TABLE 2
Change in body weights of BALB/c mice after antisense oligonucleotide
treatment (%) (all sequence numbers of WO2012/145697)

| Treatment | Body weight |
|---|---|
| PBS | 9 |
| SEQ ID NO: 320 | 9 |
| SEQ ID NO: 83 | 11 |
| SEQ ID NO: 224 | 9 |
| SEQ ID NO: 88 | 10 |
| SEQ ID NO: 103 | 14 |
| SEQ ID NO: 20 | 11 |
| SEQ ID NO: 116 | 10 |
| SEQ ID NO: 187 | 14 |
| SEQ ID NO: 210 | 12 |
| SEQ ID NO: 212 | 16 |
| SEQ ID NO: 226 | 12 |
| SEQ ID NO: 24 | 8 |
| SEQ ID NO: 39 | 9 |
| SEQ ID NO: 46 | 21 |
| SEQ ID NO: 50 | 14 |
| SEQ ID NO: 140 | 10 |
| SEQ ID NO: 17 | 10 |
| SEQ ID NO: 27 | 15 |
| SEQ ID NO: 40 | 16 |
| SEQ ID NO: 74 | 19 |

TABLE 3
Change in organ weights of BALB/c mice after antisense oligonucleotide
treatment (%) (all sequence numbers of WO2012/145697)

| Treatment | Liver | Kidney | Spleen |
|---|---|---|---|
| PBS | — | — | — |
| SEQ ID NO: 320 | 3 | −3 | −9 |
| SEQ ID NO: 83 | 10 | 1 | 13 |
| SEQ ID NO: 224 | 19 | −3 | 4 |
| SEQ ID NO: 88 | −4 | −7 | 9 |
| SEQ ID NO: 103 | 1 | −16 | 23 |
| SEQ ID NO: 20 | 12 | −4 | 9 |
| SEQ ID NO: 116 | 7 | −2 | 14 |
| SEQ ID NO: 187 | 5 | −6 | 7 |
| SEQ ID NO: 210 | 7 | −6 | 0 |
| SEQ ID NO: 212 | 12 | −7 | 5 |
| SEQ ID NO: 226 | 8 | 0 | 3 |
| SEQ ID NO: 24 | 17 | 14 | 200 |
| SEQ ID NO: 39 | −4 | −9 | 3 |
| SEQ ID NO: 46 | 18 | −9 | 79 |
| SEQ ID NO: 50 | 6 | −6 | 2 |
| SEQ ID NO: 140 | 0 | −2 | 15 |
| SEQ ID NO: 17 | 2 | 1 | 8 |
| SEQ ID NO: 27 | 5 | −2 | 58 |
| SEQ ID NO: 40 | 12 | −8 | 7 |
| SEQ ID NO: 74 | 20 | −8 | 49 |

### Liver Function

To evaluate the effect of ISIS oligonucleotides on hepatic function, plasma concentrations of transaminases were measured using an automated clinical chemistry analyzer (Hitachi Olympus AU400e, Melville, N.Y.). Plasma concentrations of ALT (alanine transaminase) and AST (aspartate transaminase) were measured and the results are presented in Table 4 expressed in IU/L. Plasma levels of cholesterol and triglycerides were also measured using the same clinical chemistry analyzer and the results are also presented in Table 4.

TABLE 4
Effect of antisense oligonucleotide treatment
on metabolic markers in the liver of BALB/c
mice (all sequence numbers of WO2012/145697)

| | ALT | AST | Cholesterol | Triglycerides |
|---|---|---|---|---|
| Treatment | (IU/L) | (IU/L) | (mg/dL) | (mg/dL) |
| PBS | 37 | 58 | 114 | 238 |
| SEQ ID NO: 320 | 36 | 57 | 114 | 234 |
| SEQ ID NO: 83 | 43 | 56 | 121 | 221 |
| SEQ ID NO: 224 | 53 | 76 | 118 | 327 |
| SEQ ID NO: 88 | 68 | 103 | 117 | 206 |
| SEQ ID NO: 103 | 136 | 152 | 144 | 168 |
| SEQ ID NO: 20 | 281 | 194 | 119 | 188 |
| SEQ ID NO: 116 | 67 | 70 | 123 | 226 |
| SEQ ID NO: 187 | 113 | 111 | 135 | 249 |
| SEQ ID NO: 210 | 56 | 63 | 128 | 234 |
| SEQ ID NO: 212 | 79 | 83 | 122 | 347 |
| SEQ ID NO: 226 | 78 | 175 | 112 | 214 |
| SEQ ID NO: 24 | 111 | 166 | 61 | 175 |
| SEQ ID NO: 39 | 635 | 508 | 110 | 179 |
| SEQ ID NO: 46 | 92 | 113 | 118 | 131 |
| SEQ ID NO: 50 | 38 | 89 | 97 | 176 |
| SEQ ID NO: 140 | 159 | 229 | 85 | 173 |
| SEQ ID NO: 17 | 90 | 87 | 86 | 222 |
| SEQ ID NO: 27 | 61 | 88 | 79 | 239 |
| SEQ ID NO: 40 | 70 | 95 | 124 | 214 |
| SEQ ID NO: 74 | 1247 | 996 | 161 | 167 |

### Kidney Function

To evaluate the effect of ISIS oligonucleotides on kidney function, plasma concentrations of blood urea nitrogen (BUN) were measured using an automated clinical chemistry analyzer (Hitachi Olympus AU400e, Melville, N.Y.). Results are presented in Table 5, expressed in mg/dL.

TABLE 5
Effect of antisense oligonucleotide treatment on kidney markers
of BALB/c mice (all sequence numbers of WO2012/145697)

| | BUN |
|---|---|
| Treatment | (mg/dL) |
| PBS | 29 |
| SEQ ID NO: 320 | 29 |
| SEQ ID NO: 83 | 28 |
| SEQ ID NO: 224 | 30 |
| SEQ ID NO: 88 | 30 |
| SEQ ID NO: 103 | 30 |
| SEQ ID NO: 20 | 29 |
| SEQ ID NO: 116 | 28 |
| SEQ ID NO: 187 | 29 |
| SEQ ID NO: 210 | 27 |
| SEQ ID NO: 212 | 26 |
| SEQ ID NO: 226 | 26 |
| SEQ ID NO: 24 | 25 |
| SEQ ID NO: 39 | 23 |
| SEQ ID NO: 46 | 28 |
| SEQ ID NO: 50 | 25 |
| SEQ ID NO: 140 | 24 |
| SEQ ID NO: 17 | 27 |
| SEQ ID NO: 27 | 27 |
| SEQ ID NO: 40 | 25 |
| SEQ ID NO: 74 | 22 |

### Example 3

### Efficacy of MOE Gapmers Targeting HBV in Transgenic Mice

Mice harboring a HBV gene fragment (Guidotti, L. G. et al., J. Virol. 1995, 69, 6158-6169) were used. The mice were treated with antisense oligonucleotides selected from studies described above and evaluated for their efficacy in this model.

Groups of 6 mice each were injected subcutaneously twice a week for 4 weeks with 50 mg/kg of SEQ ID NO: 83, SEQ ID NO: 226, SEQ ID NO: 224, SEQ ID NO: 181, SEQ ID NO: 143, or SEQ ID NO: 145 (all sequence numbers of WO2012/145697). A control group of 10 mice was injected subcutaneously twice a week for 4 weeks with PBS. Mice were euthanized 48 hours after the last dose, and livers were harvested for further analysis.

### DNA and RNA Analysis

RNA was extracted from liver tissue for real-time PCR analysis of HBV DNA, using primer probe set RTS3370. The DNA levels were normalized to picogreen. HBV RNA samples were also assayed with primer probe set RTS3370 after RT-PCR analysis. The mRNA levels were normalized to RIBOGREEN®. The data is presented in Table 6, expressed as percent inhibition compared to the control group. As shown in Table 6, most of the antisense oligonucleotides achieved reduction of HBV DNA and RNA over the PBS control. Results are presented as percent inhibition of HBV mRNA or DNA, relative to control.

TABLE 6
Percent inhibition of HBV RNA and DNA in the liver of
transgenic mice (all sequence numbers of WO2012/145697)

| Treatment | % inhibition DNA | % inhibition RNA |
|---|---|---|
| SEQ ID NO: 83 | 39 | 5 |
| SEQ ID NO: 226 | 84 | 77 |
| SEQ ID NO: 224 | 83 | 73 |
| SEQ ID NO: 181 | 56 | 28 |
| SEQ ID NO: 143 | 82 | 29 |
| SEQ ID NO: 145 | 54 | 30 |

### Rationale for Choice of the Animal Models for Examples Including Vaccine Treatments:

HLA.A2/DR1 mice (transgenic for the human HLA-A2 and HLA-DR1 molecules) were used to evaluate the ability of the candidate vaccine to induce HBc-specific CD8⁺ T-cell responses. HBV specific CD4⁺ T-cells and antibodies were evaluated in the same HLA.A2/DR1 mice.

The animal models available to assess the efficacy of a therapeutic vaccine are limited as HBV naturally infects only chimpanzees and humans. Mouse models have been developed where the whole HBV genome is expressed either through the integration of the viral genome in the host genome (HBV transgenic mice) or through infection with replicative HBV DNA, or vectors expressing the HBV genome. Although these do not reproduce the chronic HBV pathogenesis, viral replicative intermediates and proteins can be detected in the liver, and immune tolerance is observed.

The AAV2/8-HBV-transduced HLA.A2/DR1 murine model recapitulates virological and immunological characteristics of chronic HBV infection and was selected [Dion, 2013; Martin, 2015]

### Materials and Methods for Examples Involving Vaccine Treatments:

### Doses of AS01 Adjuvant System Used in the Non-Clinical Immunogenicity Studies

The AS01_{B-4} Adjuvant System is composed of immuno-enhancers QS-21 (a triterpene glycoside purified from the bark of Quillaja saponaria) and MPL (3-D Monophosphoryl lipid A), with liposomes as vehicles for these immuno-enhancers and sorbitol. In particular, a single human dose of AS01_{B-4} (0.5 mL) contains 50 μg of QS-21 and 50 μg of MPL. 1/10^{th} of a human dose i.e. 50 μl is the volume injected in mice (corresponding to 5 μg QS-21 and MPL).

### Cellular Immune Response—Intracellular Cytokine Staining (ICS)

Fresh pools of splenocytes or liver infiltrating lymphocytes collected at different time points, were stimulated ex vivo for 6 hours with pools of 15-mers, overlapping of 11aa, covering the HBc or HBs sequence. The HBc and HBs-specific cellular responses were evaluated by ICS measuring the amount of CD4⁺ or CD8 ⁺ T-cells expressing IFN-γ and/or IL-2 and/or tumor necrosis factor (TNF)-α. The technical acceptance criteria to take into account ICS results include the minimal number of acquired CD8⁺ T or CD4⁺ T cells being >3000 events.

### Humoral Immune Response—Enzyme-Linked Immunosorbent Assay (ELISA)

HBc- and HBs-specific antibody responses were measured by ELISA on sera from immunized mice at different time points. Briefly, 96-well plates were coated with HBc or HBs antigens. Individual serum samples were then added in serial dilutions and incubated for 2 hours. A biotinylated anti-mouse F(ab)′2 fragment was then added and the antigen-antibody complex was revealed by incubation with a streptavidin horseradish peroxidase complex and a peroxidase substrate ortho-phenylenediamine dihydrochloride/H₂O₂. For each time point and each antigen (HBc, HBs), an analysis of variance (ANOVA) model was fitted on log 10 titres including group, study and interaction as fixed effects and using a heterogeneous variance model (identical variances were not assumed between groups). This model was used to estimate geometric means (and their 95% CIs) as well as the geometric mean ratios and their 95% CIs. As no pre-defined criteria were set, the analysis is descriptive and 95% CIs of ratios between groups were computed without adjustment for multiplicity.

### ALT/AST Measure

The levels of ALT and AST in mouse sera were quantified using the following commercial kits:
Alanine Aminotransferase Activity Assay Kit Sigma Aldrich Cat #MAK052
Aspartate Aminotransferase Activity Assay Kit Sigma Aldrich Cat #MAK055

### Serum HBs Antigen Quantification

The circulating HBs antigen in mouse sera was quantified using the Monolisa Anti-HBs PLUS from BIO-RAD (cat #72566) and an international standard (Abbott Diagnostics).

### Histopathology Analysis

The livers (one lobe per liver) were collected and preserved in 10% formaldehyde fixative. All samples for microscopic examination were trimmed based on RITA guidelines [Ruehl-Fehlert, 2003; Kittel 2004; Morawietz 2004], embedded in paraffin wax, sectioned at a thickness of approximately 4 microns and stained with H&E. Grading of histological activity (necro-inflammatory lesions) and fibrosis was performed according to the METAVIR scoring system [Bedossa, 1996; Mohamadnejad, 2010; Rammeh, 2014]. Grading of inflammatory cell foci was done according to the Desmet score, as described by Buchmann et al [Buchmann, 2013].

Statistical analysis performed in each study is detailed in the sections pertaining to each individual study.

### Example 4

### Immunogenicity Evaluation of ChAd155-hIi-HBV/MVA-HBV/HBs-HBc/AS01_{B-4} Vaccine Regimens in HLA.A2/DR1 Transgenic Mice

### Objectives

The objective of this study was to evaluate the immunogenicity of different vaccine regimens consisting of a prime/boost with ChAd155-hIi-HBV/MVA-HBV viral vectors followed by or co-administered with two doses of recombinant proteins hepatitis B core antigen (HBcAg 4 μg) with hepatitis B surface antigen (HBsAg 1 μg) and adjuvant AS01_{B-4} (written as: HBc-HBs 4-1/AS01_{B-4}).

### Study Design

The first group of mice (N=16) was immunized at Day 0 with ChAd155-hIi-HBV followed by MVA-HBV 28 days later. Two doses of HBc-HBs 4-1 μg/AS01_{B-4} were injected 14 days apart after this prime/boost viral vector regimen (Table 4). The second group of mice (N=16) was immunized at Day 0 with ChAd155-hIi-HBV and HBc-HBs 4-1/AS01_{B-4} followed 28 days later by a boost with MVA-HBV co-administered with HBc-HBs 4-1/AS01_{B}. Two subsequent co-immunizations of MVA-HBV and HBc-HBs 4-1/AS01_{B} were performed 14 days apart (Table 4). The third group of mice (N=8) was injected with NaCl as negative control. Mice were sacrificed at 7 days post second (7dpII) and post fourth immunization (7dpIV) to determine the HBc- and HBs-specific humoral (sera) and cellular immune responses (on splenocytes and liver infiltrating lymphocytes).

This study was descriptive and no statistical sample size justification and analysis were performed.

TABLE 7
Treatment groups

| Groups | Day 0 | Day 28 | Day 42 | Day 56 | Sacrifice |
|---|---|---|---|---|---|
| 1 | 10⁸ vp ChAd155-hli- | 10⁷ pfu MVA-HBV | HBc-HBs 4-1/AS01_{B-4} | HBc-HBs 4-1/AS01_{B-4} | 7dpII and |
| | HBV | | | | 7dpIV |
| 2 | 10⁸ vp ChAd155-hli- | 10⁷ pfu MVA-HBV + | 10⁷ pfu MVA-HBV + | 10⁷ pfu MVA-HBV + | 7dpII and |
| | HBV + HBc-HBs 4- | HBc-HBs 4-1/AS01_{B-4} | HBc-HBs 4-1/AS01_{B-4} | HBc-HBs 4-1/AS01_{B-4} | 7 dpIV |
| | 1/AS01_{B-4} | | | | |
| 3 | NaCl | NaCl | NaCl | NaCl | 7dpII and |
| | | | | | 7 dpIV |

### Results

### HBc- and HBs-Specific CD8⁺ T-Cell Response (Splenocytes)

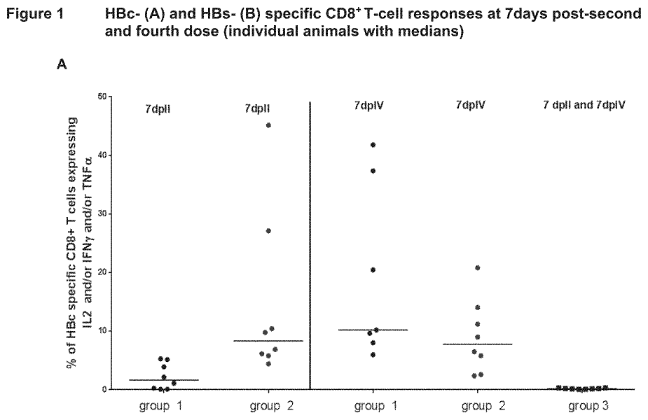

Co-administration of HBc-HBs 4-1/AS01_{B-4} with the ChAd155-hIi-HBV vector as prime and with the MVA-HBV vector as boost (Group 2) induced a 4 fold increase of HBc-specific CD8⁺ T-cell response when compared to injection of ChAd155-hIi-HBV/MVA-HBV only (Group 1) at 7dpII (FIG. 1). Similar CD8⁺ T-cell response against HBs was induced in both groups (FIG. 1).

At 7dpIV, HBc- but not HBs-specific CD8⁺ T-cell response was clearly boosted after subsequent administrations of HBc-HBs/AS01_{B-4} (5 fold increase compared to 7dpII) (Group 1). No further increase of HBc- or HBs-specific CD8⁺ T-cells was observed when two additional doses of MVA-HBV/HBc-HBs 4-1/AS01_{B-4} were co-administered (Group 2).

### HBc- and HBs-Specific CD4⁺ T-Cell Response (Splenocytes)

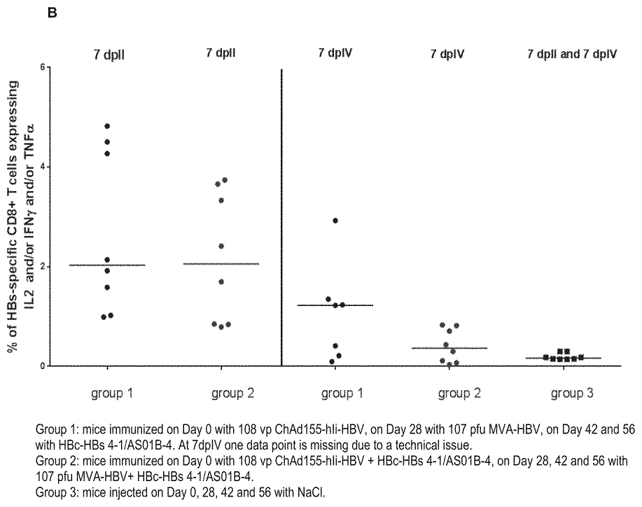

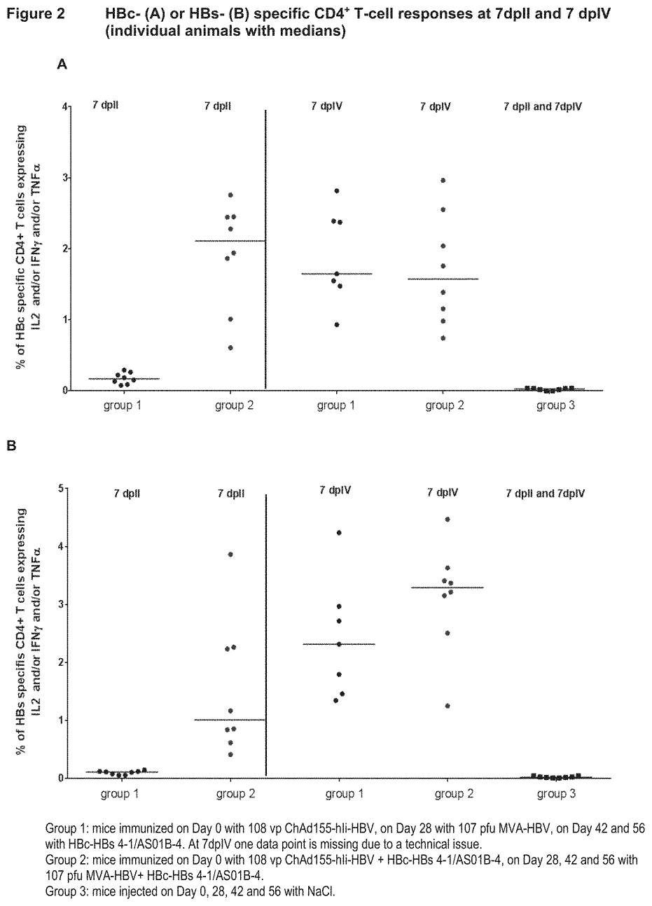

Low levels of HBc- and HBs-specific CD4⁺ T-cells were detected after prime-boost ChAd155-hIi-HBV/MVA-HBV immunization (median 0.17% and 0.11%, respectively) (Group 1) while a potent response against both antigens was observed when HBc-HBs 4-1/AS01_{B-4} was co-administered with prime-boost ChAd155-hIi-HBV/MVA-HBV (Group 2) at 7 dpII (FIG. 2).

Subsequent administrations of HBc-HBs 4-1/AS01_{B-4} after ChAd155-hIi-HBV/MVA-HBV prime-boost (Group 1) substantially enhanced both HBc- and HBs specific CD4⁺ T-cells responses (median 1.64% and 2.32%, respectively) at 7dpIV. Finally, a robust increase of HBs-specific CD4⁺ T-cells was observed when two additional doses of MVA-HBV and HBc-HBs/AS01_{B-4} were co-administered to the mice already vaccinated with the prime boost ChAd155-hIi-HBV/MVA-HBV co-administered with HBc-HBs/AS01_{B-4} (Group 2) at same time point. The HBc-specific CD4⁺ T-cells remained at the same level as at 7dpost II in that same group.

### HBc- and HBs-Specific T-Cell Responses Measured in Liver Infiltrating Lymphocytes

7 days post-last immunization, the presence of vaccine-induced T-cell responses in the liver was investigated by ICS. In order to have a sufficient number of liver infiltrating lymphocytes to perform the in vitro re-stimulation and ICS, pools of cells recovered after perfusion of 3 or 4 livers were constituted for each data point. Due to the low number of data points, no statistical analysis was performed, and the results are descriptive.

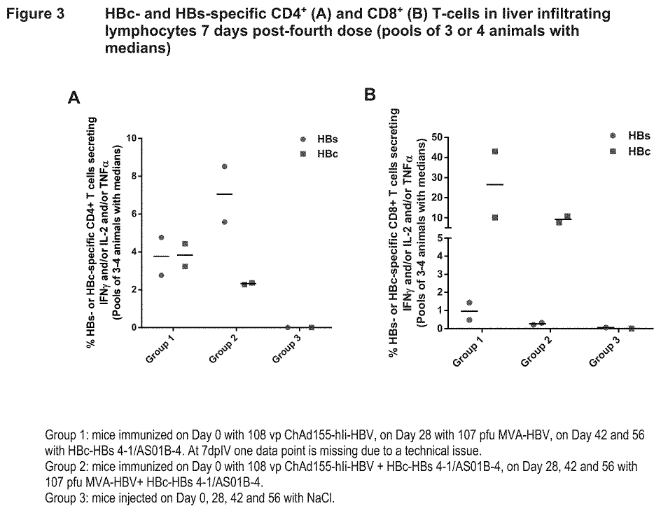

Both vaccine regimens elicited HBc- and HBs-specific CD4⁺ T-cells detectable in the liver of vaccinated mice (FIG. 3). Strong HBc-specific CD8⁺ T-cell responses were measured in the livers of animals vaccinated with both vaccine regimens, while much lower frequencies of HBs-specific CD8⁺ T-cells were measured.

### HBc- and HBs-Specific Antibody Response

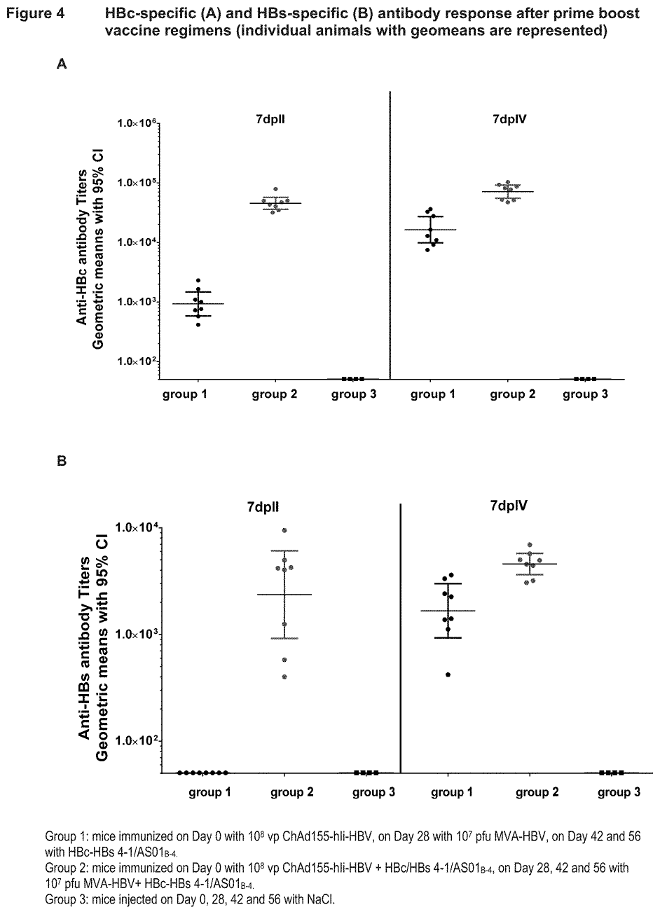

Co-administration of ChAd155-hIi-HBV/MVA-HBV with HBc-HBs 4-1/AS01_{B-4} (Group 2) induced the highest amount of anti-HBc antibodies at 7dpII (FIG. 4). Subsequent injections of MVA-HBV+HBc-HBs/AS01_{B-4} did not further increase the level of anti-HBc antibody response (7dpIV). A clear increase of anti-HBc-specific antibody response was observed at 7dpIV after injections of HBc-HBs/AS01_{B-4} in mice preliminary immunized with ChAd155-hIi-HBV and MVA-HBV (Group 1). The presence of the HBc-HBs/AS01_{B-4} component seemed to be important in the schedule to elicit potent anti-HBs antibodies as no anti-HBs antibody response was detected in animals after immunization with ChAd155-hIi-HBV/MVA-HBV (FIG. 4). The highest magnitude of response was observed in the co-ad group (Group 2) after last immunization.

### Conclusions

In HLA.A2/DR1 transgenic mice, ChAd155-hIi-HBV/MVA-HBV elicited low but detectable HBc-specific CD4⁺ T-cell responses which were clearly enhanced by HBc-HBs 4-1/AS01_{B-4}. The initial prime-boost immunization with ChAd155-hIi-HBV/MVA-HBV induced potent HBc- and HBs-specific CD8⁺ T-cell responses, with the HBc-specific responses further increased after HBc-HBs/AS01_{B-4} boost given sequentially.

Interestingly, when ChAd155-hIi-HBV/MVA-HBV were co-administered with HBc-HBs 4-1/AS01_{B-4}, high levels of HBc- and HBs-specific CD4⁺ and CD8⁺ T-cells were induced as well as antibodies after only two immunizations. Further immunizations with MVA-HBV+HBc-HBs/AS01_{B-4} did not further increase the levels of these responses.

Moreover, vaccine-induced HBc- and HBs-specific CD4⁺ and CD8⁺ T-cells were also detected in the liver of animals vaccinated with both vaccine regimens.

### Example 5

### Evaluation of the Immunogenicity and Safety of ChAd155-hIi-HBV/MVA-HBV/HBc-HBs/AS01_{B-4} Vaccine Regimens in AAV2/8-HBV Transduced HLA.A2/DR1 Mice

### Objectives

The AAV2/8-HBV-transduced HLA.A2/DR1 murine model recapitulates virological and immunological characteristics of chronic HBV infection. In this model, the liver of mice is transduced with an adeno-associated virus serotype 2/8 (AAV2/8) vector carrying a replication-competent HBV DNA genome.

A single tail vein injection of 5×10¹⁰vg (viral genome) of the AAV2/8-HBV vector leads to HBV replication and gene expression in the liver of AAV2/8-HBV-transduced mice [Dion; 2013]. HBV DNA replicative intermediates, HBV RNA transcripts and HBc antigens are detected in the liver up to 1 year post-injection without associated significant liver inflammation. HBs and HBe antigens and HBV DNA can be detected in the sera up to 1 year. Furthermore, establishment of immune tolerance to HBV antigens is observed in this surrogate model of chronic HBV infection.

The objectives of this study conducted in AAV2/8-HBV transduced HLA.A2/DR1 mice were
to demonstrate that the vaccine regimen can overcome the tolerance to HBs and HBc antigens
to evaluate the impact of liver infiltrating HBc-specific CD8⁺ T-cells, potentially targeting hepatocytes expressing the HBcAg, on the histology of the liver (H&E staining) and AST and ALT levels, as surrogate parameters for the liver function.

### Study Design

Two different vaccine regimens, based on sequential immunization with ChAd155-hIi-HBV and MVA-HBV (both encoding the HBV core [HBc] and surface [HBs] antigens), either alone or in combination with HBc-HBs 4-1/AS01_{B-4} followed by two additional doses HBc-HBs 4-1/AS01_{B-4} (either alone or in combination with MVA-HBV), were tested (Table 6).

HLA.A2/DR1 mice from groups 1, 2 and 3 were transduced with 5×10¹⁰vg of AAV2/8-HBV vector (intravenous administration) at Day 0, while Group 4 served as a positive control for immunogenicity (no establishment of tolerance prior to vaccination).

Animals from Group 1 (N=21) were immunized at Day 31 with ChAd155-hIi-HBV followed by MVA-HBV at Day 58. Two doses of HBc-HBs 4-1 μg/AS01_{B-4} were injected at Days 72 and 86 after this prime/boost viral vector regimen (Table 6).

Animals from Group 2 (N=21) were immunized at Day 31 with ChAd155-hIi-HBV and co-administrated with HBc-HBs 4-1/AS01_{B-4} followed at Day 58 by a boost with MVA-HBV co-administered with HBc-HBs 4-1/AS01_{B}. Two subsequent co-immunizations of MVA-HBV and HBc-HBs 4-1/AS01_{B} were performed at Days 72 and 86 (Table 6).

Animals from Group 3 (N=21) were injected with NaCl on Day 31, 58, 72 and 86 as negative control.

Animals from Group 4 (N=8) received the same vaccine regimen as Group 2 (except that they were not transduced with AAV2/8-HBV).

All vaccines were administered intramuscularly.

The level of HBs circulating antigen was measured in sera at Days 23, 65 and 93 (groups 1, 2 and 3).

HBs- and HBc-specific antibody responses were measured in sera from all animals at Days 23 (post-AAV2/8-HBV transduction), 65 (7 days post-second immunization) and 93 (7 days post-fourth immunization) by ELISA. The HBs- and HBc-specific CD4⁺ and CD8⁺ T cell responses were evaluated at Days 65 (9 animals/group) and 93 (12 animals/group) in splenocytes and liver infiltrating lymphocytes, after ex vivo re-stimulation and ICS (Groups 1, 2 and 3). These immunogenicity read-outs were performed only at Day 93 for animals from Group 4 (8 animals).

With regards to liver-related safety parameters, the levels of AST and ALT were measured in sera at Days 38, 65 and 93 and microscopic examination of liver sections stained with H&E was performed at Days 65 and 93 to detect potential vaccine-related histopathological changes or inflammation (Groups 1, 2 and 3).

TABLE 8
Treatment groups

| Groups | N* | Day 0 | Day 31 | Day 58 | Day 72 | Day 86 |
|---|---|---|---|---|---|---|
| 1 | 21 | AAV2/8- | 10⁸ vp ChAd155- | 10⁷ pfu MVA- | HBc-HBs 4- | HBc-HBs 4- |
| | | HBV | hli-HBV | HBV | 1/AS01_{B-4} | 1/AS01_{B-4} |
| 2 | 21 | AAV2/8- | 10⁸ vp ChAd155- | 10⁷ pfu MVA- | 10⁷ pfu MVA- | 10⁷ pfu MVA- |
| | | HBV | hli-HBV + | HBV + HBc- | HBV + HBc- | HBV + HBc- |
| | | | HBc-HBs 4- | HBs 4- | HBs 4- | HBs 4- |
| | | | 1/AS01_{B-4} | 1/AS01_{B-4} | 1/AS01_{B-4} | 1/AS01_{B-4} |
| 3 | 21 | AAV2/8- | NaCl | NaCl | NaCl | NaCl |
| | | HBV | | | | |
| 4 | 8 | No vector | 10⁸ vp ChAd155- | 10⁷ pfu MVA- | 10⁷ pfu MVA- | 10⁷ pfu MVA- |
| | | | hli-HBV + | HBV + HBc- | HBV + HBc- | HBV + HBc- |
| | | | HBc-HBs 4- | HBs 4- | HBs 4- | HBs 4- |
| | | | 1/AS01_{B-4} | 1/AS01_{B-4} | 1/AS01_{B-4} | 1/AS01_{B-4} |

*1 mouse was found dead in Group 3 before Day 65 and in Group 2 before Day 93.

### Statistical Analysis

### AST and ALT Levels

An ANOVA model for repeated measures including Gender, Day, Group and the three two-by-two interactions was fitted on the log 10-transformed enzymatic activity values, using the unstructured covariance structure. Model assumptions were verified. The interactions insignificant at the 5% level were removed from the model. For both enzymes, the final model included Gender, Day, Group and the interaction between Group and Day. The geometric means of enzymatic activity of each group at each time point were derived from this model. Group comparisons of interest are reported through geometric mean ratios (GMRs) that were also derived from this model. All these statistics are presented with a two-sided 95% confidence interval. Multiplicity was not taken into account when computing these GMRs.

All analyses were performed using SAS 9.2

### Humoral Responses

Descriptive statistics were performed to calculate the number of responders. The cut-off for responsiveness for anti-HBc or anti-HBs antibody responses was defined based on the geometric mean titers calculated in Group 3 (AAV2/8-HBV transduction but no vaccination).

### Cellular Response

Descriptive analyses were performed to define the number of responders for either HBc-, HBs-specific CD4⁺ or CD8⁺ T cells. The cut-off for responsiveness was defined as the 95^{th} percentile of measurements made in Group 3 (AAV2/8-HBV transduction but no vaccination).

### Results

HBc-Specific CD8⁺ and CD4⁺ T Cells

In AAV2/8-HBV-transduced HLA-A2/DR1 mice, the background level of HBc-specific CD8⁺ or CD4⁺ T cells was very low to undetectable without immunization at all the time-points tested (Group 3).

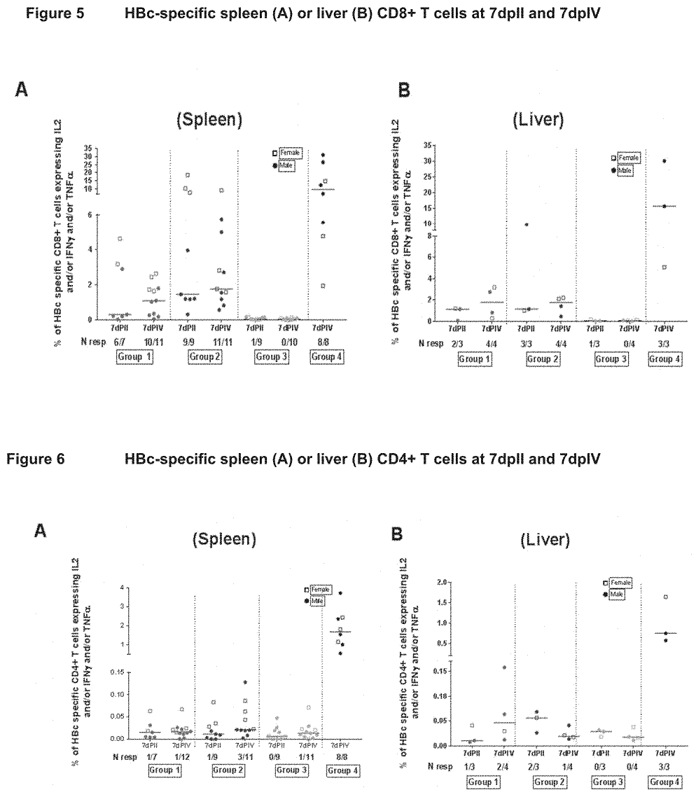

The immunization with ChAd155-hIi-HBV and MVA-HBV vectors, either alone (Group 1) or in combination with HBc-HBs 4-1/AS01_{B-4} (Group 2) induced HBc-specific CD8⁺ T cells (6/7 and 9/9 responders respectively at 7 days post-II), demonstrating a bypass of the tolerance to the HBc antigen (FIG. 5A). The two additional doses of HBc-HBs 4-1/AS01_{B-4} either alone or in combination with MVA-HBV, only modestly increased these HBc-specific CD8⁺ T cell responses as measured at 7 days post-fourth dose reaching median frequencies of 1% in Group 1 and 1.45% in Group 2. The frequencies of HBc-specific CD8⁺ T cells induced by the same vaccine regimen as in Group 2, were higher in non-transduced HLA.A2/DR1 mice from Group 4 (8/8 responders, with frequencies ˜4 fold higher at 7 days post-IV), as expected due to the immune tolerance toward the HBc antigen. HBc-specific CD8⁺ T cells were also detected in the liver of vaccinated mice, with the same profile as in spleens (FIG. 5B).

Both vaccine regimens elicited very low to undetectable HBc-specific CD4⁺ T cells in AAV2/8-HBV-transduced HLA-A2/DR1 mice (Groups 1 and 2), while a robust response was measured in non-transduced mice (Group 4), suggesting that the vaccine regimen did not overcome the CD4⁺ T cell tolerance to the HBc antigen under these experimental conditions (FIG. 6A, B).

HBs-Specific CD8⁺ and CD4⁺ T Cells

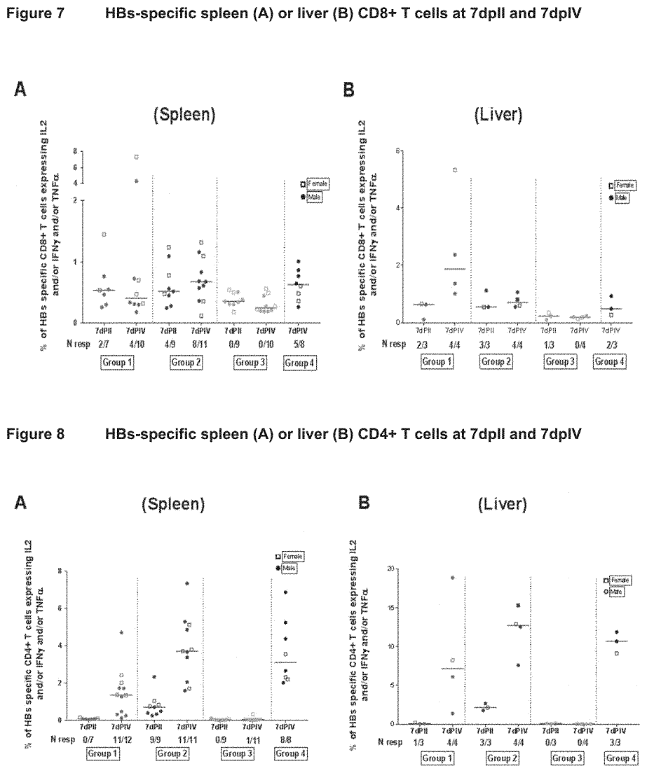

The immunization with ChAd155-hIi-HBV and MVA-HBV vectors, either alone (Group 1) or in combination with HBc-HBs 4-1/AS01_{B-4} (Group 2) elicited HBs-specific CD8⁺ T cells with no further increase of the intensities following the two additional doses of HBc-HBs 4-1/AS01_{B-4} either alone or in combination with MVA-HBV, in AAV2/8-HBV transduced mice (FIG. 7A). At the end of the vaccination schedule (7 days post-fourth dose), the frequencies of HBs-specific CD8⁺ T cells measured in the spleens of animals from Groups 1 (4/10 responders) and 2 (8/11 responders) were close to the ones detected in Group 4 (non-transduced HLA.A2/DR1 mice, median at 7 days post-IV=0.62%, 5/8 responders), suggesting an overcome of the T cell tolerance toward the HBs antigen. HBs-specific CD8⁺ T cells were detected in the livers of animals from Groups 1, 2 and 4 in most of the vaccinated animals (FIG. 7B).

HBs-specific CD4⁺ T cells were induced after administration of HBc-HBs 4-1/AS01_{B-4} alone or in combination with vectors, from 7 days post-second vaccination in Group 2 (9/9 responders) and from 7 days post-fourth vaccination in Group 1 (11/12 responders) (FIG. 8A). The vaccine schedule used in animals from Group 2 elicited about 3 fold higher frequencies of HBs-specific CD4⁺ T cells (median at 7 days post-IV=3.7%, 11/11 responders) as compared to vaccine schedule used in animals from Group 1 (median at 7 days post-IV=1.34%, 11/12 responders), reaching similar levels as in Group 4 (non-transduced HLA.A2/DR1 mice, median at 7 days post-IV=3%, 8/8 responders), suggesting an almost complete overcome of the T cell tolerance toward the HBs antigen. Similarly to the systemic CD4⁺ T cell responses, HBs-specific CD4⁺ T cells were detected in the livers of animals from Groups 1, 2 and 4 in all vaccinated animals (FIG. 8B).

### HBs- and HBc-Specific Antibody Responses

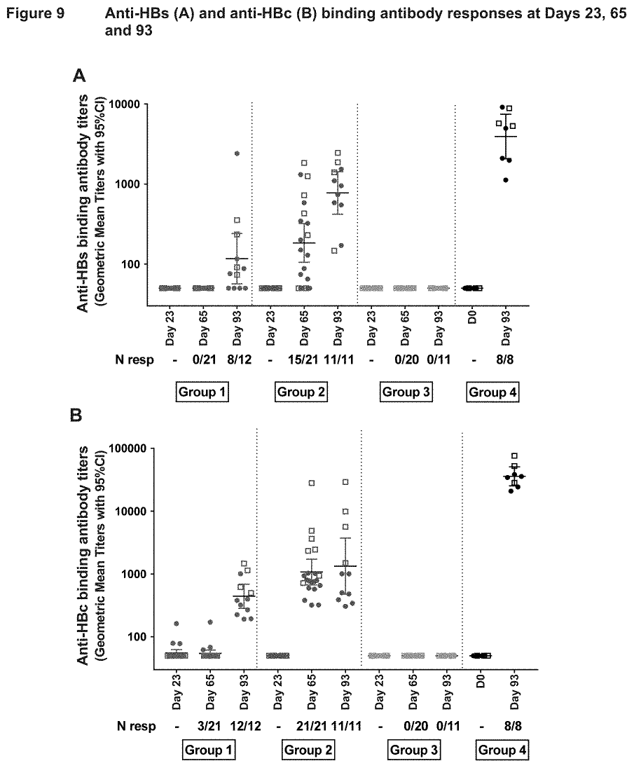

At 23 days after the injection of the AAV2/8-HBV vector, no anti-HBs antibody responses were detected in HLA.A2/DR1 mice, suggesting a strong humoral tolerance toward the HBs antigen. The immunization with ChAd155-hIi-HBV and MVA-HBV vectors alone (Group 1) did not break this tolerance while the immunization of the vectors in combination with HBc-HBs 4-1/AS01_{B-4} led to the induction of anti-HBs antibody responses in 15 out of the 21 animals at Day 65 (Group 2) (FIG. 9A). The further administration of 2 doses of HBc-HBs 4-1/AS01_{B-4} in group 1 elicited detectable anti-HBs antibodies (Geometric mean titers (GMT) of 116.8 and 8/12 responders at Day 93) and the 2 additional doses of MVA-HBV combined with HBc-HBs 4-1/AS01_{B-4} in Group 2 further increased the intensity of the anti-HBs antibody response up to a GMT of 775 with 11/11 responders, while remaining ˜5 fold lower than in non-AAV2/8-HBV transduced animals from Group 4 (GMT=3933; 8/8 responders) at Day 93.

Similarly, anti-HBc antibody responses were induced only when the HBc-HBs 4-1/AS01_{B-4} component was present in the vaccine regimen, with 3 fold higher levels measured at Day 93 in animals from Group 2 (GMT=1335.5; 11/11 responders) as compared to Group 1 (GMT=442.8; 12/12 responders) FIG. 9B). The anti-HBc antibody titers induced in the non-transduced mice (Group 4) with the same vaccine regimen as in Group 2 were higher (˜27 fold, GMT=35782; 8/8 responders).

These results show that the presence of the adjuvanted protein component in the vaccine regimen is critical to break the humoral tolerance to both HBc and HBs antigens. Furthermore the vaccine regimen used in Group 2, containing 4 administrations of the HBc-HBs 4-1/AS01_{B-4} elicited the highest anti-HBc and anti-HBs antibody responses, while remaining lower than in non-AAV2/8-HBV transduced mice (Group 4).

### AST/ALT Levels

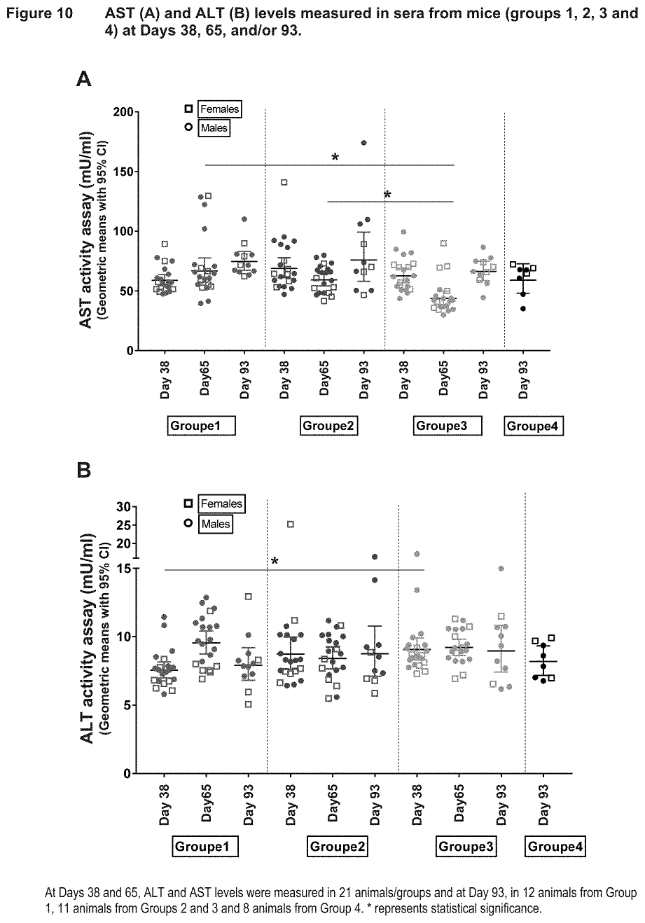

As a liver-related inflammation parameter, the serum activities of AST and ALT were measured at Days 38 (7 days post-first vaccination), 65 (7 days post-second vaccination) and/or 93 (7 days post-fourth immunization) (all Groups). Overall, the AST and ALT levels were stable during the course of the vaccine regimens (Groups 1 and 2) in AAV2/8-HBV transduced HLA.A2/DR1 mice and similar to the ones measures in mice not receiving vaccines (Group 3) (FIG. 10). AST levels were found statistically significantly higher in animals from the vaccine groups (Groups 1 and 2) as compared to the control Group 3 at Day 65. However, the AST levels were surprisingly low at Day 65 in animals from Group 3 as compared to the rest of the kinetics, suggesting that these differences were rather due to the particularly unexpectedly low values obtained in the control group 3 at this time-point, rather than an increase of the AST levels in the vaccine groups (Groups 1 and 2) (FIG. 10A).

A slightly lower ALT level was measured at Day 38 in animals from Group 1 as compared to in control animals from Group 3, but this difference was not considered as clinically relevant (FIG. 10B).

### Liver Microscopic Examination

Microscopic examination of liver sections stained with H&E was performed at Days 65 and 93 to detect potential vaccine-related histopathological changes or inflammation (Groups 1, 2 and 3) (Table 7).

There were no test item-related microscopic findings either on Day 65 (7 days after the injection of the second viral vectored vaccine, MVA-HBV with or without HBc-HBs 4-1/AS01_{B-4}) or on Day 93 (7 days after the last injection) in AAV2/8-HBV transduced HLA-A2/DR mice, i.e. there were no histopathological changes that could be associated with the use of the vaccine components ChAd155-hIi-HBV, MVA-HBV and HBc-HBs 4-1/AS01_{B-4}.

In addition, except for control animal 3.13 (which presented a focal grade 1 piecemeal necrosis), none of the animals presented morphological signs of chronic hepatitis.

Other microscopic findings noted in treated animals were considered incidental changes, as they also occurred in the control group, were of low incidence/magnitude, and/or are common background findings in mice of similar age [McInnes, 2012].

TABLE 9
Microscopic examination of the livers of animals from groups 1, 2 and 3 at Days 65 and 93

| | 45028_EPS (Raw Data) | | | | | | | | | | | | | | | | | | | | |
|---|---|---|---|---|---|---|---|---|---|---|---|---|---|---|---|---|---|---|---|---|---|
| LIVER | 1.1 | 1.2 | 1.3 | 1.4 | 1.5 | 1.6 | 1.7 | 1.8 | 1.9 | 1.10 | 1.11 | 1.12 | 1.13 | 1.14 | 1.15 | 1.16 | 1.17 | 1.18 | 1.19 | 1.20 | 1.21 |
| Day of sacrifice | 93 | 93 | 93 | 93 | 65 | 93 | 65 | 93 | 93 | 65 | 93 | 65 | 65 | 65 | 93 | 93 | 65 | 93 | 65 | 65 | 93 |
| Piecemeal | 0 | 0 | 0 | 0 | 0 | 0 | 0 | 0 | 0 | 0 | 0 | 0 | 0 | 0 | 0 | 0 | 0 | 0 | 0 | 0 | 0 |
| necrosis | | | | | | | | | | | | | | | | | | | | | |
| Focal lobular | 0 | 0 | 0 | 0 | 0 | 0 | 0 | 0 | 0 | 0 | 0 | 0 | 0 | 0 | 0 | 0 | 0 | 0 | 0 | 0 | 0 |
| necrosis | | | | | | | | | | | | | | | | | | | | | |
| METAVIR A | 0 | 0 | 0 | 0 | 0 | 0 | 0 | 0 | 0 | 0 | 0 | 0 | 0 | 0 | 0 | 0 | 0 | 0 | 0 | 0 | 0 |
| (Activity) | | | | | | | | | | | | | | | | | | | | | |
| METAVIR B | 0 | 0 | 0 | 0 | 0 | 0 | 0 | 0 | 0 | 0 | 0 | 0 | 0 | 0 | 0 | 0 | 0 | 0 | 0 | 0 | 0 |
| (Fibrosis) | | | | | | | | | | | | | | | | | | | | | |
| Inflammatory | 0 | 0 | 0 | 0 | 0 | 0 | 0 | 0 | 0 | 0 | 0 | 0 | 0 | 0 | 0 | 0 | 0 | 0 | 0 | 0 | 0 |
| cell foci | | | | | | | | | | | | | | | | | | | | | |
| Single cell | 0 | 0 | 0 | 0 | 0 | 0 | 0 | 0 | 0 | 0 | 0 | 0 | 0 | 0 | 0 | 0 | 0 | 0 | 0 | 0 | 0 |
| necrosis | | | | | | | | | | | | | | | | | | | | | |
| Extramedullary | 0 | 0 | 0 | 0 | 0 | 0 | 0 | 0 | 0 | 0 | 0 | 0 | 0 | 0 | 0 | 0 | 0 | 0 | 0 | 0 | 0 |
| hematopoiesis | | | | | | | | | | | | | | | | | | | | | |
| Pigment | 0 | 0 | 0 | 0 | 0 | 0 | 0 | 0 | 0 | 0 | 0 | 0 | 0 | 0 | 0 | 0 | 0 | 0 | 0 | 0 | 0 |
| (consistent with | | | | | | | | | | | | | | | | | | | | | |
| hemosiderin); | | | | | | | | | | | | | | | | | | | | | |
| Kupffer cells | | | | | | | | | | | | | | | | | | | | | |
| | Group 2 (“high-dose”), treated with: ChAd155-HBV (at Day 30) + | | | | | | | | | | | | | | | | | | | | |
| | MVA-HBV (at Day 58) + HBc-HBs/AS01B-4 (at Day 30, 58, 72 amd 86) | | | | | | | | | | | | | | | | | | | | |
| LIVER | 2.1 | 2.2 | 2.3 | 2.4 | 2.5 | 2.6 | 2.7 | 2.8 | 2.9 | 2.10 | 2.11 | 2.12 | 2.13 | 2.14 | 2.15 | 2.16 | 2.17 | 2.18 | 2.19 | 2.20 | 2.21 |
| Day of sacrifice | 93 | 65 | 93 | 93 | 65 | 93 | 65 | 65 | NA | 93 | 93 | 93 | 65 | 65 | 93 | 93 | 65 | 93 | 65 | 65 | 93 |
| Piecemeal | 0 | 0 | 0 | 0 | 0 | 0 | 0 | 0 | NA | 0 | 0 | 0 | 0 | 0 | 0 | 0 | 0 | 0 | 0 | 0 | 0 |
| necrosis | | | | | | | | | | | | | | | | | | | | | |
| Focal lobular | 0 | 0 | 0 | 0 | 0 | 0 | 0 | 0 | NA | 0 | 0 | 0 | 0 | 0 | 0 | 0 | 0 | 0 | 0 | 0 | 0 |
| necrosis | | | | | | | | | | | | | | | | | | | | | |
| METAVIR A | 0 | 0 | 0 | 0 | 0 | 0 | 0 | 0 | NA | 0 | 0 | 0 | 0 | 0 | 0 | 0 | 0 | 0 | 0 | 0 | 0 |
| (Activity) | | | | | | | | | | | | | | | | | | | | | |
| METAVIR B | 0 | 0 | 0 | 0 | 0 | 0 | 0 | 0 | NA | 0 | 0 | 0 | 0 | 0 | 0 | 0 | 0 | 0 | 0 | 0 | 0 |
| (Fibrosis) | | | | | | | | | | | | | | | | | | | | | |
| Inflammatory | 0 | 0 | 0 | 0 | 0 | 0 | 0 | 0 | NA | 0 | 0 | 0 | 0 | 0 | 0 | 0 | 0 | 0 | 0 | 0 | 0 |
| cell foci | | | | | | | | | | | | | | | | | | | | | |
| Single cell | 0 | 0 | 0 | 0 | 0 | 0 | 0 | 0 | NA | 1 | 0 | 0 | 0 | 0 | 0 | 0 | 0 | 0 | 0 | 0 | 0 |
| necrosis | | | | | | | | | | | | | | | | | | | | | |
| Extramedullary | 0 | 0 | 0 | 0 | 0 | 0 | 0 | 0 | NA | 0 | 0 | 0 | 0 | 0 | 1 | 1 | 0 | 0 | 0 | 0 | 0 |
| hematopoiesis | | | | | | | | | | | | | | | | | | | | | |
| Pigment | 0 | 0 | 0 | 0 | 0 | 0 | 0 | 0 | NA | 0 | 0 | 0 | 0 | 0 | 0 | 0 | 0 | 0 | 1 | 0 | 0 |
| (consistent with | | | | | | | | | | | | | | | | | | | | | |
| hemosiderin); | | | | | | | | | | | | | | | | | | | | | |
| Kupffer cells | | | | | | | | | | | | | | | | | | | | | |
| | Group 3 (control), treated with: NaCl | | | | | | | | | | | | | | | | | | | | |
| LIVER | 3.1 | 3.2 | 3.3 | 3.4 | 3.5 | 3.6 | 3.7 | 3.8 | 3.9 | 3.10 | 3.11 | 3.12 | 3.13 | 3.14 | 3.15 | 3.16 | 3.17 | 3.18 | 3.19 | 3.20 | 3.21 |
| Day of sacrifice | 65 | NA | 65 | 93 | 93 | 65 | 93 | 65 | 65 | 65 | 93 | 93 | 93 | 93 | 93 | 93 | 65 | 93 | 65 | 65 | 93 |
| Piecemeal | 0 | NA | 0 | 0 | 0 | 0 | 0 | 0 | 0 | 0 | 0 | 0 | 1* | 0 | 0 | 0 | 0 | 0 | 0 | 0 | 0 |
| necrosis | | | | | | | | | | | | | | | | | | | | | |
| Focal lobular | 0 | NA | 0 | 0 | 0 | 0 | 0 | 0 | 0 | 0 | 0 | 0 | 0 | 0 | 0 | 0 | 0 | 0 | 0 | 0 | 0 |
| necrosis | | | | | | | | | | | | | | | | | | | | | |
| METAVIR A | 0 | NA | 0 | 0 | 0 | 0 | 0 | 0 | 0 | 0 | 0 | 0 | 1 | 0 | 0 | 0 | 0 | 0 | 0 | 0 | 0 |
| (Activity) | | | | | | | | | | | | | | | | | | | | | |
| METAVIR B | 0 | NA | 0 | 0 | 0 | 0 | 0 | 0 | 0 | 0 | 0 | 0 | 0 | 0 | 0 | 0 | 0 | 0 | 0 | 0 | 0 |
| (Fibrosis) | | | | | | | | | | | | | | | | | | | | | |
| Inflammatory | 0 | NA | 0 | 0 | 0 | 0 | 0 | 0 | 0 | 0 | 0 | 0 | 0 | 0 | 0 | 0 | 0 | 0 | 0 | 0 | 0 |
| cell foci | | | | | | | | | | | | | | | | | | | | | |
| Single cell | 0 | NA | 0 | 0 | 0 | 0 | 0 | 0 | 1 | 0 | 0 | 0 | 0 | 0 | 0 | 0 | 0 | 0 | 0 | 0 | 0 |
| necrosis | | | | | | | | | | | | | | | | | | | | | |
| Extramedullary | 0 | NA | 0 | 0 | 0 | 0 | 0 | 0 | 0 | 0 | 0 | 0 | 0 | 0 | 0 | 0 | 0 | 0 | 0 | 0 | 0 |
| hematopoiesis | | | | | | | | | | | | | | | | | | | | | |
| Pigment | 0 | NA | 0 | 0 | 0 | 1 | 0 | 0 | 0 | 0 | 0 | 0 | 0 | 0 | 0 | 0 | 0 | 0 | 0 | 0 | 0 |
| (consistent with | | | | | | | | | | | | | | | | | | | | | |
| hemosiderin); | | | | | | | | | | | | | | | | | | | | | |
| Kupffer cells | | | | | | | | | | | | | | | | | | | | | |

NA: not applicable (mortality 2.9)
*focal/slight piecemeal necrosis in a single portal space.
NA: not applicable (mortality 3.2)

HBs Antigen Levels in sera from AAV2/8-HBV Injected Mice.

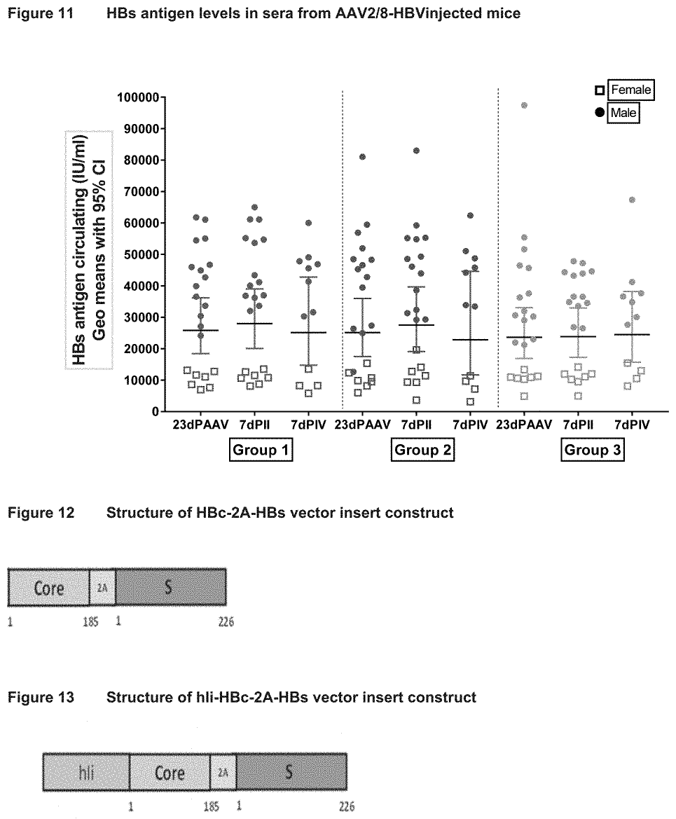

As already reported in Dion et al [Dion, 2013], HBs antigen levels were higher in males as compared to females, 23 days post-injection with the AAV2/8-HBV vectors. These levels remained stable in all groups, without detectable impact of the vaccination regimens (FIG. 11). AAV2/8-HBV injected mouse is however not an animal model for studying vaccine efficacy on HBsAg.

### Conclusion

In a surrogate model of chronic HBV infection where immune tolerance toward HBc and HBs antigen is established, i.e. AAV2/8-HBV-transduced HLA-A2/DR1 mice, both tested vaccine regimens bypassed the tolerance by inducing HBc- and HBs-specific IgG and CD8⁺ T cell responses as well as HBs-specific CD4⁺ T cell responses, albeit at lower levels than in non-transduced mice, as expected due to strong immune tolerance. When the ChAd155-hIi-HBV/MVA-HBV vectors were co-administered with HBc-HBs 4-1/AS01_{B-4}, the intensities of the vaccine induced antibody and T cell responses were higher than with the vaccine regimen where the vectors and adjuvanted proteins were administered sequentially. Furthermore, while assessing the vaccine-associated liver inflammation by measuring serum activities of AST and ALT and by performing liver histopathological evaluation, no increase in liver enzymes was detected in the vaccine groups when compared with the non-vaccinated one and no microscopic findings could be related to the vaccine treatments. Altogether, these results show that the tested vaccine candidates successfully restored HBs- and HBc-specific antibody and CD8⁺ T cell responses as well as HBs-specific CD4⁺ T cell responses without detection of associated-signs of liver alteration, under these experimental conditions.

### Example 6

### Evaluation of the Efficacy, Immunogenicity and Safety of HBV ASO/ChAd155-hIi-HBV/MVA-HBV/HBc-HBs/AS01_{B} Regimens in AAV2/8-HBV Transduced HLA.A2/DR1 Mice

### Objectives

The study utilises the AAV2/8-HBV-transduced HLA.A2/DR1 murine model of chronic HBV infection as described in Example 5.

The objectives of this study are:
to demonstrate that the combination of HBV ASO with the vaccine regimens can further overcome the tolerance to HBs (anti-HBs Ab titres) as compared to vaccine regimen alone
to demonstrate that the combination of HBV ASO with the vaccine regimens can reduce circulating HBs antigen level as compared to vaccine regimen alone
to assess the HBc-specific CD8⁺ T cell responses to the combination of HBV ASO with the vaccine regimens
To assess the impact of the combination of HBV ASO with the vaccine regimens on serum HBV DNA viral load
to evaluate AST and ALT levels, as surrogate parameters for the liver function.

### Study Design

Two different vaccine regimens, based on sequential immunisation with ChAd155-hIi-HBV and MVA-HBV (both encoding the HBV core [HBc] and surface [HBs] antigens), either alone or in combination with HBc-HBs 4-1/AS01_{B} followed by two additional doses HBc-HBs 4-1/AS01_{B} (either alone or in combination with MVA-HBV), are tested with or without treatment with HBV ASO (Table 10).

HLA.A2/DR1 mice in groups 1 to 6 are transduced with 5×10¹⁰vg of AAV2/8-HBV vector (intravenous administration, tail vein) at Day 0, while Group 7 serves as a positive control for safety and immunogenicity of the vaccine regimens (no HBV ASO treatment and no establishment of tolerance prior to treatment).

Animals from Groups 1 to 6 are pre-treated with HBV ASO (SEQ ID NO: 226 of WO2012/145697)) or NaCl on Days 30, 33 and 37, then this treatment continues weekly, concurrently with administration of the specified vaccine regimen (or NaCl) to Day 100.

Animals from Groups 1 and 2, treated with HBV ASO or NaCl respectively, are immunized at Day 44 with ChAd155-hIi-HBV followed by MVA-HBV at Day 72. Two doses of HBc-HBs 4-1 μg/AS01_{B} are administered at Days 86 and 100, after this prime/boost viral vector regimen (Table 10).

Animals from Groups 3 and 4, treated with HBV ASO or NaCl respectively, are immunized at Day 44 with ChAd155-hIi-HBV co-administered with HBc-HBs 4-1/AS01_{B} followed at Day 72 by a boost of MVA-HBV co-administered with HBc-HBs 4-1/AS01_{B}. Two subsequent co-immunizations of MVA-HBV and HBc-HBs 4-1/AS01_{B} are performed at Days 86 and 100 (Table 10).

Animals from Groups 5 and 6, treated with NaCl or HBV ASO respectively, are injected with NaCl on Days 44, 72, 86 and 100 as a negative control for the vaccine regimes.

All components of the regimens are administered intramuscularly.

The levels of serum HBsAg and serum HBV DNA are measured at Days 0 (before induction of the CHB model), 21 (to confirm induction of the CHB model) 44, 58, 72, 79, 86, 100, 107, 114, 128, and 142

HBs- and HBc-specific antibody responses are measured in sera from all animals at Days 0, 21, 44, 58, 72, 79, 86, 100, 107, 114, 128, and 142 by ELISA.

The groups of mice are split for sacrifice and evaluation of HBs- and HBc-specific CD4⁺ and CD8⁺ T cell responses (ICS—spleen and perfused liver) at Days 79 (groups 1-4 and group 7), 107 and 142 (all groups).

With regards to liver-related safety parameters, the levels of AST and ALT enzymes are measured in sera at Days 0, 44, 58, 86, 100, 114, 128 and 142.

TABLE 10
Treatment groups

| | | Day 30, 33, 37 & | | | | | |
|---|---|---|---|---|---|---|---|
| | | once per week to | | | | | |
| Group | Day 0 | Day 100 | Day 44 | Day 72 | Day 86 | Day 100 | Sacrifice |
| 1 | AAV2/8- | HBV ASO | 10⁸ vp ChAd155-hIi- | 10⁷ pfu MVA-HBV | HBc-HBs 4-1/ | HBc-HBs 4-1/ | Day 79, |
| | HBV | | HBV | | AS01_{B} | AS01_{B} | Day 107, |
| | | | | | | | Day 142 |
| 2 | AAV2/8- | NaCl | 10⁸ vp ChAd155-hIi- | 10⁷ pfu MVA-HBV | HBc-HBs 4-1/ | HBc-HBs 4-1/ | Day 79, |
| | HBV | | HBV | | AS01_{B} | AS01_{B} | Day 107, |
| | | | | | | | Day 142 |
| 3 | AAV2/8- | HBV ASO | 10⁸ vp ChAd155-hIi- | 10⁷ pfu MVA- | 10⁷ pfu MVA- | 10⁷ pfu MVA- | Day 79, |
| | HBV | | HBV + HBc-HBs 4- | HBV + HBc-HBs 4- | HBV + HBc-HBs 4- | HBV + HBc-HBs 4- | Day 107, |
| | | | 1/AS01_{B} | 1/AS01_{B} | 1/AS01_{B} | 1/AS01_{B} | Day 142 |
| 4 | AAV2/8- | NaCl | 10⁸ vp ChAd155-hIi- | 10⁷ pfu MVA- | 10⁷ pfu MVA- | 10⁷ pfu MVA- | Day 79, |
| | HBV | | HBV + HBc-HBs 4- | HBV + HBc-HBs 4- | HBV + HBc-HBs 4- | HBV + HBc-HBs 4- | Day 107, |
| | | | 1/AS01_{B} | 1/AS01_{B} | 1/AS01_{B} | 1/AS01_{B} | Day 142 |
| 5 | AAV2/8- | NaCl | NaCl | NaCl | NaCl | NaCl | Day 107, |
| | HBV | | | | | | Day 142 |
| 6 | AAV2/8- | HBV ASO | NaCl | NaCl | NaCl | NaCl | Day 107, |
| | HBV | | | | | | Day 142 |
| 7 | No vector | — | 10⁸ vp ChAd155-hIi- | 10⁷ pfu MVA- | 10⁷ pfu MVA- | 10⁷ pfu MVA- | Day 79, |
| | | | HBV + HBc-HBs 4- | HBV + HBc-HBs 4- | HBV + HBc-HBs 4- | HBV + HBc-HBs 4- | Day 107, |
| | | | 1/AS01_{B} | 1/AS01_{B} | 1/AS01_{B} | 1/AS01_{B} | Day 142 |

### Example 7

### Evaluation of the Efficacy, Immunogenicity and Safety of HBV-ASO/ChAd155-hIi-HBV/MVA-HBV/HBc-HBs/AS01_{B} Regimens in AAV2/8-HBV Transduced HLA.A2/DR1 Mice

### Objectives

The study utilises the AAV2/8-HBV-transduced HLA.A2/DR1 murine model of chronic HBV infection as described in Example 5.

The objectives of this study are identical to those of Example 6:
to demonstrate that the combination of HBV ASO with the vaccine regimens can further overcome the tolerance to HBs (anti-HBs Ab titres) as compared to vaccine regimen alone
to demonstrate that the combination of HBV ASO with the vaccine regimens can reduce circulating HBs antigen level as compared to vaccine regimen alone
to assess the HBc-specific CD8⁺ T cell responses to the combination of HBV ASO with the vaccine regimens
to assess the impact of the combination of HBV ASO with the vaccine regimens on serum HBV DNA viral load
to evaluate AST and ALT levels, as surrogate parameters for the liver function and also to perform histopathological examination of major organs (liver, lung, heart, brain, kidney, thymus), for the evaluation of the potential systemic toxicity.

### Study Design

Two different vaccine regimens, based on sequential immunisation with ChAd155-hIi-HBV and MVA-HBV (both encoding the HBV core [HBc] and surface [HBs] antigens), either alone or in combination with HBc-HBs 4-1/AS01_{B} followed by two additional doses HBc-HBs 4-1/AS01_{B} (either alone or in combination with MVA-HBV), are tested with or without treatment with HBV ASO (Table 11). In addition, the treatment with HBV ASO either stops before administration of the first vaccine on day 44, or continues until day 100.

HLA.A2/DR1 mice in groups 1 to 6 and 8 to 10 are transduced with 10¹⁰vg of AAV2/8-HBV vector (intravenous administration, tail vein) at Day 0, while Group 7 serves as a positive control for safety and immunogenicity of the vaccine regimens (no HBV ASO treatment and no establishment of tolerance prior to treatment).

Animals from Groups 1, 6, and 8 are pre-treated with HBV ASO (SEQ ID NO: 226 of WO2012/145697) on Days 31, 35 and 38. Then this treatment continues weekly, concurrently with administration of the specified vaccine regimen (or NaCl) to Day 100.

Animals from Groups 3, 4 and 10 are also pre-treated with HBV ASO (SEQ ID NO: 226 of WO2012/145697) on Days 31, 35 and 38. However, an additional HBV ASO administration takes place on day 42 and then treatment with HBV ASO is stopped.

Animals from Groups 2, 5 and 9 are pre-treated with or NaCl on Days 31, 35 and 38, then this treatment continues weekly, concurrently with administration of the specified vaccine regimen (or NaCl) to Day 100.

Animals from Groups 1, 2 and 3, treated with HBV ASO or NaCl, are immunized at Day 44 with ChAd155-hIi-HBV followed by MVA-HBV at Day 72. Two doses of HBc-HBs 4-1 μg/AS01_{B} are administered at Days 86 and 100, after this prime/boost viral vector regimen (Table 11).

Animals from Groups 8, 9 and 10, treated with HBV ASO or NaCl, are immunized at Day 44 with ChAd155-hIi-HBV co-administered with HBc-HBs 4-1/AS01_{B} followed at Day 72 by a boost of MVA-HBV co-administered with HBc-HBs 4-1/AS01_{B}. Two subsequent co-immunizations of MVA-HBV and HBc-HBs 4-1/AS01_{B} are performed at Days 86 and 100 (Table 11).

Animals from Groups 4, 5 and 6, treated with NaCl or HBV ASO, are injected with NaCl on Days 44, 72, 86 and 100 as a negative control for the vaccine regimes.

All components of the regimens are administered intramuscularly.

The levels of serum HBsAg and serum HBV DNA are measured at Days 0 (before induction of the CHB model), 21 (to confirm induction of the CHB model) 42, 56, 70, 80, 84, 98, 107, 113, 127, and 141.

HBs- and HBc-specific antibody responses are measured in sera from all animals at Days 0, 21, 42, 56, 70, 80, 84, 98, 107, 113, 127, and 141 by ELISA.

The groups of mice are split for sacrifice and evaluation of HBs- and HBc-specific CD4⁺ and CD8⁺ T cell responses (ICS—spleen and perfused liver) at Days 80 (groups 1, 2, 3 and group 7), 107 and 141 (all groups).

With regards to liver-related safety parameters, the levels of AST and ALT enzymes are measured in sera at least at days Days 0, 42, 80, 107, and 141.

TABLE 11
Treatment groups

| | | **Day 31, | | | | | |
|---|---|---|---|---|---|---|---|
| | | 35, 38 | | | | | |
| | | & once | | | | | |
| | | per week | | | | | |
| Group | Day 0 | to Day 100 | Day 44 | Day 72 | Day 86 | Day 100 | Sacrifice |
| 1 | AAV2/8- | HBV ASO** | 10⁸ vp ChAd155- | 10⁷ pfu MVA-HBV | HBc-HBs 4- | HBc-HBs 4- | 7dPII (Day 80) |
| | HBV | | hIi-HBV | | μg/AS01_{B-4} | μg/AS01_{B-4} | 7dPIV (Day 107) |
| | | | | | | | 41PIV (Day 141) |
| 2 | AAV2/8- | NaCl** | 10⁸ vp ChAd155- | 10⁷ pfu MVA-HBV | HBc-HBs 4- | HBc-HBs 4- | 7dPII (Day 80) |
| | HBV | | hIi-HBV | | μg/AS01_{B-4} | μg/AS01_{B-4} | 7dPIV (Day 107) |
| | | | | | | | 41PIV (Day 141) |
| 3 | AAV2/8- | HBV ASO | 10⁸ vp ChAd155- | 10⁷ pfu MVA-HBV | HBc-HBs 4- | HBc-HBs 4- | 7dPII (Day 80) |
| | HBV | Only at Days | hIi-HBV | | μg/AS01_{B-4} | μg/AS01_{B-4} | 7dPIV (Day 107) |
| | | 31, 35, | | | | | 41PIV (Day 141) |
| | | 38 & day 42 | | | | | |
| 4 | AAV2/8- | HBV ASO | NaCl | NaCl | NaCl | NaCl | 41PIV (Day 141) |
| | HBV | Only at Days | | | | | |
| | | 31, 35, | | | | | |
| | | 38 & day 42 | | | | | |
| 5 | AAV2/8- | NaCl** | NaCl | NaCl | NaCl | NaCl | 41PIV (Day 141) |
| | HBV | | | | | | |
| 6 | AAV2/8- | HBV ASO** | NaCl | NaCl | NaCl | NaCl | 41PIV (Day 141) |
| | HBV | | | | | | |
| 7 | No vector | — | 10⁸ vp ChAd155- | 10⁷ pfu MVA-HBV | HBc-HBs 4- | HBc-HBs 4- | 7dPII (Day 80) |
| | | | hIi-HBV | | μg/AS01_{B-4} | μg/AS01_{B-4} | 7dPIV (Day 107) |
| | | | | | | | 41PIV (Day 141) |
| 8 | AAV2/8- | HBV ASO** | 10⁸ vp ChAd155-hIi- | 10⁷ pfu MVA- | 10⁷ pfu MVA- | 10⁷ pfu MVA- | 41PIV (Day 141) |
| | HBV | | HBV + HBc-HBs 4- | HBV + HBc-HBs 4- | HBV + HBc-HBs 4- | HBV + HBc-HBs 4- | |
| | | | 1 μg/AS01_{B-4} | 1 μg/AS01_{B-4} | 1 μg/AS01_{B-4} | 1 μg/AS01_{B-4} | |
| 9 | AAV2/8- | NaCl** | 10⁸ vp ChAd155-hIi- | 10⁷ pfu MVA- | 10⁷ pfu MVA- | 10⁷ pfu MVA- | 41P1V (Day 141) |
| | HBV | | HBV + HBc-HBs 4- | HBV + HBc-HBs 4- | HBV + HBc-HBs 4- | HBV + HBc-HBs 4- | |
| | | | 1 μg/AS01_{B-4} | 1 μg/AS01_{B-4} | 1 μg/AS01_{B-4} | 1 μg/AS01_{B-4} | |
| 10 | AAV2/8- | HBV ASO | 10⁸ vp ChAd155-hIi- | 10⁷ pfu MVA- | 10⁷ pfu MVA- | 10⁷ pfu MVA- | 42P1V (Day 142) |
| | HBV | Only at Days | HBV + HBc-HBs 4- | HBV + HBc-HBs 4- | HBV + HBc-HBs 4- | HBV + HBc-HBs 4- | |
| | | 31, 35, | 1 μg/AS01_{B-4} | 1 μg/AS01_{B-4} | 1 μg/AS01_{B-4} | 1 μg/AS01_{B-4} | |
| | | 33 & day 42 | | | | | |

SEQUENCE LISTINGS

SEQ ID NO: 1: Amino acid sequence of HBs
MENITSGFLGPLLVLQAGFFLLTRILTIPQSLDSWWTSLNFLGGSPVCLGQNSQSPTSNHSPTSCPPICPGYRWM
CLRRFIIFLFILLLCLIFLLVLLDYQGMLPVCPLIPGSTTTNTGPCKTCTTPAQGNSMFPSCCCTKPTDGNCTCIPIP
SSWAFAKYLWEWASVRFSWLSLLVPFVQWFVGLSPTVWLSAIWMMWYWGPSLYSIVSPFIPLLPIFFCLWVYI
SEQ ID NO: 2: Amino acid sequence of HBc truncate
MDIDPYKEFGATVELLSFLPSDFFPSVRDLLDTASALYREALESPEHCSPHHTALRQAILCWGELMTLATWVGNN
LEDPASRDLVVNYVNTNMGLKIRQLLWFHISCLTFGRETVLEYLVSFGVWIRTPPAYRPPNAPILSTLPETTVV
SEQ ID NO: 3: Amino acid sequence of spacer incorporating 2A cleaving region of the foot and
mouth disease virus
APVKQTLNFDLLKLAGDVESNPGP
SEQ ID NO: 4: Nucleotide sequence encoding spacer incorporating 2A cleavage region of the foot
and mouth disease virus
GCCCCTGTGAAGCAGACCCTGAACTTCGACCTGCTGAAGCTGGCCGGCGACGTGGAGAGCAATCCCGGCCCT
SEQ ID NO: 5: Amino acid sequence of HBc-2A-HBs
MDIDPYKEFGATVELLSFLPSDFFPSVRDLLDTASALYREALESPEHCSPHHTALRQAILCWGELMTLATWVGNN
LEDPASRDLVVNYVNTNMGLKIRQLLWFHISCLTFGRETVLEYLVSFGVWIRTPPAYRPPNAPILSTLPETTVVRR
RDRGRSPRRRTPSPRRRRSQSPRRRRSQSRESQCAPVKQTLNFDLLKLAGDVESNPGPMENITSGFLGPLLVLQ
AGFFLLTRILTIPQSLDSWWTSLNFLGGSPVCLGQNSQSPTSNHSPTSCPPICPGYRWMCLRRFIIFLFILLLCLIF
LLVLLDYQGMLPVCPLIPGSTTTNTGPCKTCTTPAQGNSMFPSCCCTKPTDGNCTCIPIPSSWAFAKYLWEWAS
VRFSWLSLLVPFVQWFVGLSPTVWLSAIWMMWYWGPSLYSIVSPFIPLLPIFFCLWVYI
SEQ ID NO: 6: Nucleotide sequence encoding HBc-2A-HBs
ATGGACATCGATCCCTACAAGGAATTTGGCGCCACCGTGGAGCTGCTGAGCTTCCTGCCCAGCGACTTCTTC
CCCAGCGTGAGGGACCTCCTGGACACCGCCAGCGCCCTGTACAGGGAGGCCCTGGAATCTCCCGAGCACTG
CAGCCCACACCACACCGCACTGAGGCAGGCCATCCTGTGCTGGGGAGAGCTGATGACCCTCGCCACCTGGGT
GGGCAACAACCTGGAGGACCCCGCCAGCAGGGACCTGGTGGTGAACTACGTCAACACCAACATGGGCCTGA
AGATCAGGCAGCTGCTGTGGTTCCACATCAGCTGCCTGACCTTCGGCAGGGAGACCGTGCTGGAGTACCTG
GTGAGCTTCGGCGTGTGGATCAGGACACCTCCCGCCTACAGACCCCCCAACGCCCCCATCCTGAGCACCCTG
CCCGAGACCACAGTGGTGAGGAGGAGGGACAGGGGCAGGTCACCCAGGAGGAGGACTCCAAGCCCCAGGAG
GAGGAGGAGCCAGAGCCCCAGGAGAAGGAGGAGCCAGAGCAGGGAGAGCCAGTGCGCCCCTGTGAAGCAG
ACCCTGAACTTCGACCTGCTGAAGCTGGCCGGCGACGTGGAGAGCAATCCCGGCCCTATGGAGAACATCACC
AGCGGCTTCCTGGGCCCCCTGCTGGTGCTGCAGGCAGGCTTCTTCCTGCTGACCAGGATCCTGACCATCCCC
CAGAGCCTGGACAGCTGGTGGACCAGCCTGAACTTCCTCGGCGGGAGCCCCGTGTGCCTGGGCCAGAACAG
CCAGTCTCCCACCAGCAATCACAGCCCCACCAGCTGCCCCCCAATCTGTCCTGGCTACCGGTGGATGTGCCT
GAGGAGGTTCATCATCTTCCTGTTCATCCTGCTCCTGTGCCTGATCTTCCTGCTGGTGCTGCTGGACTACCA
GGGAATGCTGCCAGTGTGTCCCCTGATCCCCGGCTCAACCACCACTAACACCGGCCCCTGCAAAACCTGCAC
CACCCCCGCTCAGGGCAACAGCATGTTCCCAAGCTGCTGCTGCACCAAGCCCACCGACGGCAACTGCACCTG
CATTCCCATCCCCAGCAGCTGGGCCTTCGCCAAGTATCTGTGGGAGTGGGCCAGCGTGAGGTTCAGCTGGCT
CAGCCTGCTGGTGCCCTTCGTCCAGTGGTTTGTGGGCCTGAGCCCCACCGTGTGGCTGAGCGCCATCTGGAT
GATGTGGTACTGGGGCCCCAGCCTGTACTCCATCGTGAGCCCCTTCATCCCCCTGCTGCCCATTTTCTTCTG
CCTGTGGGTGTACATC
SEQ ID NO: 7: Amino add sequence of hIi
MHRRRSRSCREDQKPVMDDQRDLISNNEQLPMLGRRPGAPESKCSRGALYTGFSILVTLLLAGQATTAYFLYQQ
QGRLDKLTVTSQNLQLENLRMKLPKPKPVSKMRMATPLLMQALPMGALPQGPMQNATKYGNMTEDHVMHLLQ
NADPLKVYPPLKSFPENLRHLKNTMETIDWKVFESWMHHWLLFEMSRHSLEQKPTDAPPKESLELEDPSSGGVT
KQDLGPVPM
SEQ ID NO: 8: Nucleotide sequence encoding hIi
atgcacaggaggaggagcaggagctgcagggaggaccagaagcccgtgatggacgaccagcgcgacctgatcagcaacaacgagcagc
tgccaatgctgggcaggaagcccggagcacccgaaagcaagtgcagcaggggcaccctgtacaccggcttcagcatcctggtgaccctcct
gctggccggccaggccaccaccgcctatttcctgtaccagcagcagggcaggctcgataagctgaccgtgacctcccagaacctgcagctgg
agaacctgaggatgaagctgcccaagccceccaagcccgtgagcaagatgaggatggccacccccctgctgatgcaggctctgcccatggg
ggccctgccccagggccccatgcagaacgccaccaaatacgacaacatgaccgaggaccacgtaatgcacctgctgcagaacgccgatcct
ctgaaggtgtacccacccctgaaaggcagcttccccgagaacctcaggcacctgaagaacaccatggagaccatcgactggaaggtgttcga
gagctggatgcaccactggctgctgttcgagatgagccggcacagcctggagcagaagcccaccgacgcccctcccaaggagagcctcgag
ctcgaggacccaagcagcggcctgggcgtgaccaagcaggacctgggccccgtgcccatg
SEQ ID NO: 9: Amino acid sequence of hIi-HBc-2A-HBs
MHRRRSRSCREDQKPVMDDQRDLISNNEQLPMLGRRPGAPESKCSRGALYTGFSILVTLLLAGQATTAYFLYQQ
QGRLDKLTVTSQNLQLENLRMKLPKPKPVSKMRMATPLLMQALPMGALPQGPMQNATKYGNMTEDHVMHLLQ
NADPLKVYPPLKSFPENLRHLKNTMETIDWKVFESWMHHWLLFEMSRHSLEQKPTDAPPKESLELEDPSSGGVT
KQDLGPVPMMDIDPYKEFGATVELLSFLPSDFFPSVRDLLDTASALYREALESPEHCSPHHTALRQAILCWGELM
TLATWVGNNLEDPASRDLVVNYVNTNMGLKIRQLLWFHISCLTFGRETVLEYLVSFGVWIRTPPAYRPPNAPILS
TLPETTVVAPVKQTLNFDLLKLAGDVESNPGPMENITSGFLGPLLVLQAGFFLLTRILTIPQSLDSWWTSLNFLGG
SPVCLGQNSQSPTSNHSPTSCPPICPGYRWMCLRRFIIFLFILLLCLIFLLVLLDYQGMLPVCPLIPGSTTTNTGPC
KTCTTPAQGNSMFPSCCCTKPTDGNCTCIPIPSSWAFAKYLWEWASVRFSWLSLLVPFVQWFVGLSPTVWLSAT
WMMWYWGPSLYSIVSPFIPLLPIFFCLWVYI
SEQ ID NO: 10: Nucleotide sequence encoding hIi-HBc-2A-HBs
ATGCACAGGAGGAGGAGCAGGAGCTGCAGGGAGGACCAGAAGCCCGTGATGGACGACCAGCGCGACCTGAT
CAGCAACAACGAGCAGCTGCCAATGCTGGGCAGGAGGCCCGGAGCACCCGAAAGCAAGTGCAGCAGGGGCG
CCCTGTACACCGGCTTCAGCATCCTGGTGACCCTCCTGCTGGCCGGCCAGGCCACCACCGCCTATTTCCTGT
ACCAGCAGCAGGGCAGGCTCGATAAGCTGACCGTGACCTCCCAGAACCTGCAGCTGGAGAACCTGAGGATG
AAGCTGCCCAAGCCCCCCAAGCCCGTGAGCAAGATGAGGATGGCCACCCCCCTGCTGATGCAGGCTCTGCCC
ATGGGGGCCCTGCCCCAGGGCCCCATGCAGAACGCCACCAAATACGGCAACATGACCGAGGACCACGTGATG
CACCTGCTGCAGAACGCCGATCCTCTGAAGGTGTACCCACCCCTGAAAGGCAGCTTCCCCGAGAACCTCAGG
CACCTGAAGAACACCATGGAGACCATCGACTGGAAGGTGTTCGAGAGCTGGATGCACCACTGGCTGCTGTTC
GAGATGAGCCGGCACAGCCTGGAGCAGAAGCCCACCGACGCCCCTCCCAAGGAGAGCCTCGAGCTCGAGGA
CCCAAGCAGCGGCCTGGGCGTGACCAAGCAGGACCTGGGCCCCGTGCCCATGGACATTGACCCCTACAAGG
AGTTCGGCGCCACCGTCGAACTGCTGAGCTTCCTCCCCAGCGACTTCTTCCCCTCCGTGAGGGATCTGCTGG
ACACAGCTAGCGCCCTGTACAGGGAGGCCCTGGAGAGCCCCGAGCACTGCAGCCCCCACCACACAGCCCTGA
GGCAGGCCATCCTCTGTTGGGGCGAGCTGATGACCCTGGCCACCTGGGTGGGCAATAACCTGGAGGACCCC
GCCAGCAGGGACCTGGTGGTCAACTACGTGAACACCAACATGGGCCTGAAGATCAGGCAGCTGCTGTGGTT
CCACATCAGCTGCCTGACCTTTGGCAGGGAGACCGTCCTGGAGTACCTGGTGAGCTTCGGCGTGTGGATCA
GGACTCCCCCAGCCTACAGGCCCCCTAACGCCCCCATCCTGTCTACCCTGCCCGAGACCACCGTGGTGAGGA
GGAGGGACAGGGGCAGAAGCCCCAGGAGAAGGACCCCTAGCCCCAGGAGGAGGAGGAGCCAGAGCCCCAG
GAGGAGGAGGAGCCAGAGCCGGGAGAGCCAGTGCGCCCCTGTGAAGCAGACCCTGAACTTCGACCTGCTGA
AGCTGGCCGGCGACGTGGAGAGCAATCCCGGCCCTATGGAAAACATCACCAGCGGCTTCCTGGGCCCCCTGC
TGGTGCTGCAGGCCGGCTTCTTCCTGCTGACCAGGATCCTGACCATTCCCCAGTCACTGGACAGCTGGTGGA
CCAGCCTGAACTTCCTCGGCGGGAGCCCCGTGTGCCTGGGCCAGAATAGCCAGAGCCCCACCAGCAACCACT
CTCCCACTTCCTGCCCCCCTATCTGCCCCGGCTACAGGTGGATGTGCCTGAGGAGGTTCATCATCTTCCTGT
TCATCCTGCTGCTGTGCCTGATCTTCCTGCTGGTGCTGCTGGACTACCAGGGAATGCTGCCCGTGTGTCCCC
TGATCCCCGGAAGCACCACCACCAACACCGGCCCCTGCAAGACCTGCACCACCCCCGCCCAGGGCAACTCTA
TGTTCCCCAGCTGCTGCTGCACCAAGCCCACCGACGGCAACTGCACTTGCATTCCCATCCCCAGCAGCTGGG
CCTTCGCCAAATATCTGTGGGAGTGGGCCAGCGTGAGGTTTAGCTGGCTGAGCCTGCTGGTGCCTTCGTG
CAGTGGTTTGTGGGCCTGAGCCCCACCGTGTGGCTGAGCGCCATCTGGATGATGIGGTACTGGGGCCCCTC
CCTGTACAGCATCGTGAGCCCCTTCATCCCCCTCCTGCCCATCTTCTTCTGCCTGTGGGTGTACATC
SEQ ID NO: 11: Amino acid sequence of HBc
MDIDPYKEFGATVELLSFLPSDFFPSVRDLLDTASALYREALESPEHCSPHHTALRQAILCWGELMTLATWGNN
LEDPASRDLVVNYVNTNMGLKIRQLLWFHISCLTFGRETVLEYLVSFGVWIRTPPAYRPPNAPILSTLPETTVVRR
RDRGRSPRRRTPSPRRRRSQSPRRRRSQSRESQC
SEQ ID NO: 12: Amino add sequence of hii alternate variant
MHRRRSRSCREDQKPVMDDQRDLISNNEQLPMLGRRPGAPESKCSRGALYTGFSILVTLLLAGQATTAYFLYQQ
QGRLDKLTVTSQNLQLENLRMKLPKPPKPVSKMRMATPLLMQALPMGALPQGPMQNATKYGNMTEDHVMHLL
QNADPLKVYPPLKGSFPENLRHLKNTMETIDWKVFESWMHHWLLFEMSRHSLEQKPTDAPPKESLELEDPSSGL
GVTKQDLGPVP
SEQ ID NO: 13: Nucleotide sequence encoding hI alternate variant
ATGCACAGGAGGAGAAGCAGGAGCTGTCGGGAAGATCAGAAGCCAGTCATGGATGACCAGCGCGACCTTAT
CTCCAACAATGAGCAACTGCCCATGCTGGGCCGGCGCCCTGGGGCCCCGGAGAGCAAGTGCAGCCGCGGAG
CCCTGTACACAGGCTTTTCCATCCTGGTGACTCTGCTCCTCGCTGGCCAGGCCACCACCGCCTACTTCCTGTA
CCAGCAGCAGGGCCGGCTGGACAAACTGACAGTCACCTCCCAGAACCTGCAGCTGGAGAACCTGCGCATGAA
GCTTCCCAAGCCTCCCAAGCCTGTGAGCAAGATGCGCATGGCCACCCCGCTGCTGATGCAGGCGCTGCCCAT
GGGAGCCCTGCCCCAGGGGCCCATGCAGAATGCCACCAAGTATGGCAACATGACAGAGGACCATGTGATGC
ACCTGCTCCAGAATGCTGACCCCCTGAAGGTGTACCCGCCACTGAAGGGGAGCTTCCCGGAGAACCTGAGAC
ACCTTAAGAACACCATGGAGACCATAGACTGGAAGGTCTTTGAGAGCTGGATGCACCATTGGCTCCTGTTTG
AAATGAGCAGGCACTCCTTGGAGCAAAAGCCCACTGACGCTCCACCGAAAGAGTCACTGGAACTGGAGGACC
CGTCTTCTGGGCTGGGTGTGACCAAGCAGGATCTGGGCCCAGTCCCC
SEQ ID NO: 14: Alternative nucleic acid sequence of hIi-HBc-2A-HBs
ATGCACAGGAGGAGAAGCAGGAGCTGTCGGGAAGATCAGAAGCCAGTCATGGATGACCAGCGCGACCTTAT
CTCCAACAATGAGCAACTGCCCATGCTGGGCCGGCGCCCTGGGGCCCCGGAGAGCAAGTGCAGCCGCGGAG
CCCTGTACACAGGCTTTTCCATCCTGGTGACTCTGCTCCTCGCTGGCCAGGCCACCACCGCCTACTTCCTGTA
CCAGCAGCAGGGCCGGCTGGACAAACTGACAGTCACCTCCCAGAACCTGCAGCTGGAGAACCTGCGCATGAA
GCTTCCCAAGCCTCCCAAGCCTGTGAGCAAGATGCGCATGGCCACCCCGCTGCTGATGCAGGCGCTGCCCAT
GGGAGCCCTGCCCCAGGGGCCCATGCAGAATGCCACCAAGTATGGCAACATGACAGAGGACCATGTGATGC
ACCTGCTCCAGAATGCTGACCCCCTGAAGGTGTACCCGCCACTGAAGGGGAGCTTCCCGGAGAACCTGAGAC
ACCTTAAGAACACCATGGAGACCATAGACTGGAAGGTCTTGAGAGCTGGATGCACCATTGGCTCCTGTTTG
AAATGAGCAGGCACTCCITGGAGCAAAAGCCCACTGACGCTCCACCGAAAGAGTCACTGGAACTGGAGGACC
CGTCTTCTGGGCTGGGTGTGACCAAGCAGGATCTGGGCCCAGTCCCCATGGACATTGACCCTTATAAAGAAT
TTGGAGCTACTGTGGAGTTACTCTCGTTTTTGCCTTCTGACTTCTTTCCTTCCGTCAGAGATCTCCTAGACAC
CGCCTCAGCTCTGTATCGAGAAGCCTTAGAGTCTCCTGAGCATTGCTCACCTCACCATACTGCACTCAGGCAA
GCCATTCTCTGCTGGGGGGAATTGATGACTCTAGCTACCTGGGTGGGTAATAATTTGGAAGATCCAGCATCC
AGGGATCTAGTAGTCAATTATGTTAATACTAACATGGGTITAAAGATCAGGCAACTATTGTGGTTTCATATAT
CTTGCCTTACTTTTGGAAGAGAGACTGTACTTGAATATTTGGTCTTTCGGAGTGTGGATTCGCACTCCTCC
AGCCTATAGACCACCAAATGCCCCTATCTTATCAACACTTCCGGAAACTACTGTTGTTAGACGACGGGACCGA
GGCAGGTCCCCTAGAAGAAGAACTCCCTCGCCTCGCAGACGCAGATCTCAATCGCCGCGTCGCAGAAGATCT
CAATCTCGGGAATCTCAATGTGCCCCTGTGAAGCAGACCCTGAACTTCGACCTGCTGAAGCTGGCCGGCGAC
GTGGAGAGCAATCCCGGCCCTATGGAGAACATCACATCAGGATTCCTAGGACCCCTGCTCGTGTTACAGGCG
GGGTTTTTCTTGTTGACAAGAATCCTCACAATACCGCAGAGTCTAGACTCGTGGTGGACTTCTCTCAATTTTC
TAGGGGGATCACCCGTGTGTCTTGGCCAAAATTCGCAGTCCCCAACCTCCAATCACTCACCAACCTCCTGTCC
TCCAATTTGTCCTGGTTATCGCTGGATGTGTCTGCGGCGTTTTATCATATTCCTCTTCATCCTGCTGCTATGC
CTCATCTTCTTATTGGTTCTTCTGGATTATCAAGGTATGTTGCCCGTTTGTCCTCTAATTCCAGGATCAACAA
CAACCAATACGGGACCATGCAAAACCTGCACGACTCCTGCTCAAGGCAACTCTATGTTTCCCTCATGTTGCTG
TACAAAACCTACGGATGGAAATTGCACCTGTATTCCCATCCCATCGTCCTGGGCTTTCGCAAAATACCTATGG
GAGTGGGCCTCAGTCCGTTTCTCTTGGCTCAGTTTACTAGTGCCATTTGTTCAGTGGTTCGTAGGGCTTTCC
CCCACTGTTTGGCTTTCAGCTATATGGATGATGTGGTATTGGGGGCCAAGTCTGTACAGCATCGTGAGTCCC
TTTATACCGCTGTTACCAATTTTCTTTTGTCTCTGGGTATACATT
SEQ ID NO: 15: Alternative amino add sequence of hIi-H8c-2A-HBs
MHRRRSRSCREDQKPVMDDQRDLISNNEQLPMLGRRPGAPESKCSRGALYTGFSILVTLLLAGQATTAYFLYQQ
QGRLDKLTVTSQNLQLENLRMKLPKPPKPVSKMRMATPLLMQALPMGALPQGPMQNATKYGNMTEDHVMHLL
QNADPLKVYPPLKGSFPENLRHLKNTMETIDWKVFESWMHHWLLFEMSRHSLEQKPTDAPPKESLELEDPSSGL
GVTKQDLGPVPMDIDPYKEFGATVELLSFLPSDFFPSVRDLLDTASALYREALESPEHCSPHHTALRQAILCWGEL
MTLATWVGNNLEDPASRDLVVNYVNTNMGLKIRQLLWFHISCLTFGRETVLEYLVSFGVWIRTPPAYRPPNAPIL
STLPETTVVRRRDRGRSPRRRTPSPRRRRSQSPRRRRSQSRESQCAPVKQTLNFDLLKLAGDVESNPGPMENIT
SGFLGPLLVLQAGFFLLTRILTIPQSLDSWWTSLNFLGGSPVCLGQNSQSPTSNHSPTSCPPICPGYRWMCLRRF
IIFLFILLLCLIFLLVLLDYQGMLPVCPLIPGSTTTNTGPCKTCTTPAQGNSMFPSCCCTKPTDGNCTCIPIPSSWAF
AKYLWEWASVRFSWLSLLVPFVQWFVGLSPTVWLSAIWMMWYWGPSLYSIVSPFIPLLPIFFCLWVYI
SEQ ID NO: 16: Nucleotide sequence of Hepatitis B viral genome (GENBANK Accession No.
U95551.1)
aattccacaa cctttcacca aactctgcaa gatcccagag tgagaggcct gtatttccct
gctggtggct ccagttcagg agcagtaaac cctgttccga ctactgcctc tcccttatcg
tcaatcttct cgaggattgg ggaccctgcg ctgaacatgg agaacatcac atcaggattc
ctaggacccc ttctcgtgtt acaggcgggg tttttcttgt tgacaagaat cctcacaata
ccgcagagtc tagactcgtg gtggacttct ctcaattttc tagggggaac taccgtgtgt
cttggccaaa attcgcagtc cccaacctcc aatcactcac caacctcctg tcctccaact
tgtcctggtt atcgctggat gtgtctgcgg cgttttatca tcttcctctt catcctgctg
ctatgcctca tcttcttgtt ggttcttctg gactatcaag gtatgttgcc cgtttgtcct
ctaattccag gatcctcaac caccagcacg ggaccatgcc gaacctgcat gactactgct
caaggaacct ctatgtatcc ctcctgttgc tgtaccaaac cttcggacgg aaattgcacc
tgtattccca tcccatcatc ctgggctttc ggaaaattcc tatgggagtg ggcctcagcc
cgttttccct ggctcagttt actagtgcca tttgttcagt ggttcgtagg gctttccccc
actgtttggc tttcagttat atggatgatg tggtattggg ggccaagtct gtacagcatc
ttgagtccct ttttaccgct gttaccaatt ttcttttgtc tttgggtata catttaaacc
ctaacaaaac aaagagatgg ggttactctc tgaattttat gggttatgtc attggaagtt
atgggtcctt gccacaagaa cacatcatac aaaaaatcaa agaatgtttt agaaaacttc
ctattaacag gcctattgat tggaaagtat gtcaacgaat tgtgggtctt ttgggttttg
ctgccccatt tacacaatgt ggttatcctg cgttaatgcc cttgtatgca tgtattcaat
ctaagcaggc tttcactttc tcgccaactt acaaggcctt tctgtgtaaa caatacctga
acctttaccc cgttgcccgg caacggccag gtctgtgcca agtgtttgct gacgcaaccc
ccactggctg gggcttggtc atgggccaic agcgcgtgcg tggaaccttt tcggctcctc
tgccgatcca tactgcggaa ctcctagccg cttgttttgc tcgcagcagg tctggagcaa
acattatcgg gactgataac tctgttgtcc tctcccgcaa atatacatcg tatccatggc
tgctaggctg tgctgccaac tggatcctgc gcgggacgtc ctttgtttac gtcccgtcgg
cgctgaatcc tgcggacgac ccttctcggg gtcgcttggg actctctcgt ccccttctcc
gtctgccgtt ccgaccgacc acggggcgca cctctcttta cgcggactcc ccgtctgtgc
cttctcatct gccggaccgt gtgcacttcg cttcacctct gcacgtcgca tggagaccac
cgtgaacgcc caccgaatgt tgcccaaggt cttacataag aggactcttg gactctctgc
aatgtcaacg accgaccttg aggcatactt caaagactgt ttgtttaaag actgggagga
gttgggggag gagattagat taaaggtctt tgtactagga ggctgtaggc ataaattggt
ctgcgcacca gcaccatgca actttttcac ctctgcctaa tcatctcttg ttcatgtcct
actgttcaag cctccaagct gtgccttggg tggctttggg gcatggacat cgacccttat
aaagaatttg gagctactgt ggagttactc tcgtttttgc cttctgactt ctttccttca
gtacgagatc ttctagatac cgcctcagct ctgtatcggg aagccttaga gtctcctgag
cattgttcac ctcaccatac tgcactcagg caagcaattc tttgctgggg ggaactaatg
actctagcta cctgggtggg tgttaatttg gaagatccag catctagaga cctagtagtc
agttatgtca acactaatat gggcctaaag ttcaggcaac tcttgtggtt tcacatttct
tgtctcactt ttggaagaoa aaccgttata gagtatttgg tgtctttcgg agtgtggatt
cgcactcctc cagcttatag accaccaaat gcccctatcc tatcaacact tccggaaact
actgttgtta gacgacgagg caggtcccct agaagaagaa ctccctcgcc tcgcagacga
aggtctcaat cgccgcgtcg cagaagatct caatctcggg aacctcaatg ttagtattcc
ttggactcat aaggtgggga actttactgg tctttattct tctactgtac ctgtctttaa
tcctcattgg aaaacaccat cttttcctaa tatacattta caccaagaca ttatcaaaaa
atgtgaacaa tttgtaggcc cacttacagt taatgagaaa agaagattgc aattgattat
gcctgctaga ttttatccaa aggttaccaa atatttacca ttgaataagg gtattaaacc
ttattatcca gaacatctag ttaatcatta cttccaaact agacactatt tacacactct
atggaaggcg ggtatattat ataagagaaa aacaacacat agcgcctcat tttgtgggtc
accatattct tgggaacaag atctacagca tggggcagaa tctttccacc agcaatcctc
tgggattctt tcccgaccac cagttggatc cagccttcag agcaaacaca gcaaatccag
attgggactt caatcccaac aaggacacct ggccagacgc caacaaggta ggaactggag
cattcgggct gggtttcacc ccaccgcacg gaggcctttt ggggtggagc cctcaggctc
agggcatact acaaactttg ccagcaaatc cgcctcctgc ctccaccaat cgccagacag
gaaggcagcc taccccgctg tctccacctt tgagaaacac tcatcctcag gccatgcagt gg

### REFERENCES

Al-Mahtab M, Akbar S M, Aguilar J C, Uddin M H, Khan M S, Rahman S. Therapeutic potential of a combined hepatitis B virus surface and core antigen vaccine in patients with chronic hepatitis B. Hepatol Int 2013; 7(4):981-9.

Bedossa P, Poynard T, An algorithm for the grading of activity in chronic hepatitis C. The METAVIR Cooperative Study Group. Hepatology. 1996; 24(2):289-93.

Bertoletti A, Ferrari C. Innate and adaptive immune responses in chronic hepatitis B virus infections: towards restoration of immune control of viral infection. Gut. 2012; 61(12):1754-64.

Block T M, Locarnini S, McMahon B J, Rehermann B, Peters M G. Use of Current and New Endpoints in the Evaluation of Experimental Hepatitis B Therapeutics. Clin Infect Dis. 2017; 64(9):1283-1288.

Boni C, Laccabue D, Lampertico P, et al. Restored function of HBV-specific T cells after long-term effective therapy with nucleos(t)ide analogues. Gastroenterology. 2012; 143(4):963-73.e9.

Borghese F, Clanchy F I. CD74: an emerging opportunity as a therapeutic target in cancer and autoimmune disease. Expert Opin Ther Targets. 2011; 15(3):237-51.

Buchmann P, Dembek C, Kuklick L, et al. Novel therapeutic hepatitis B vaccine induces cellular and humoral immune responses and breaks tolerance in hepatitis B virus (HBV) transgenic mice. Vaccine. 2013; 31(8):1197-203.

Cavenaugh J S, Awi D, Mendy M, et al. Partially Randomized, Non-Blinded Trial of DNA and MVA Therapeutic Vaccines Based on Hepatitis B Virus Surface Protein for Chronic HBV Infection. PLoS ONE 2011; 6:1-14.

Capone S, Naddeo M, D'Alise A M, et al. Fusion of HCV nonstructural antigen to MHC class II-associated invariant chain enhances T-cell responses induced by vectored vaccines in nonhuman primates. Mol Ther. 2014; 22(5):1039-47.

Cornberg M, Wong V W, Locarnini S, Brunette M, Janssen H L, Chan H L. The role of quantitative hepatitis B surface antigen revisited. J Hepatol. 2017; 66(2):398-411.

Di Lullo, G., E. Soprana, M. Panigada, A. Palini, A. Agresti, C. Comunian, A. Milani, I. Capua, V. Erfle and A. G. Siccardi (2010). The combination of marker gene swapping and fluorescence-activated cell sorting improves the efficiency of recombinant modified vaccinia virus Ankara vaccine production for human use. Journal of Virological Methods 163(2): 195-204

Dion S, Bourgine M, Godon O, Levillayer F, Michel M L. Adeno-associated virus-mediated gene transfer leads to persistent hepatitis B virus replication in mice expressing HLA-A2 and HLA-DR1 molecules. J Virol. 2013; 87(10):5554-63.

Donnelly M L, Luke G, Mehrotra A, Li X, Hughes L E, Gani D, Ryan M D. Analysis of the aphthovirus 2A/2B polyprotein ‘cleavage’ mechanism indicates not a proteolytic reaction, but a novel translational effect: a putative ribosomal ‘skip’. J Gen Virol. 2001 May; 82 (Pt 5):1013-25

Durantel D, Zoulim F. New antiviral targets for innovative treatment concepts for hepatitis B virus and hepatitis delta virus. J Hepatol 2016; 64; S117-S131.

EASL 2017. Clinical Practice Guidelines on the management of hepatitis B virus infection. J Hepatol 2017; 67(2), 370-398.

Fontaine H, Kahi S, Chazallon C, et al. Anti-HBV DNA vaccination does not prevent relapse after discontinuation of analogues in the treatment of chronic hepatitis B: a randomised trial—ANRS HB02 VAC-ADN. Gut. 2015; 64:139-147.

Hilgers et al., Synergistic Effects of Synthetic Adjuvants on the Humoral immune Response. Int. Arch. Allergy. Immunol. 1986, 79(4):392-6;

Hilgers et al., Novel adjuvants for humoral immune responses. Immunology, 1987, 60(1):141-6

Jung M C, Grüner N, Zachoval R, et al. Immunological monitoring during therapeutic vaccination as a prerequisite for the design of new effective therapies: induction of a vaccine-specific CD4+ T-cell proliferative response in chronic hepatitis B carriers. Vaccine. 2002; 20(29-30):3598-612.

Kensil C R et al. Separation and characterization of saponins with adjuvant activity from Quillaja saponaria Molina cortex. J. Immunology (1991) 146: 431-437.

Kensil C R, Saponins as Vaccine Adjuvants. Crit. Rev. Ther. Drug Carrier Syst., 1996, 13:1-55;

Kittel B, Ruehl-Fehlert C, Morawietz G, et al. Revised guides for organ sampling and trimming in rats and mice—Part 2. A joint publication of the RITA and NACAD groups. Exp Toxicol Pathol. 2004; 55(6):413-31.

Kranidioti H, Manolakopoulos S, Khakoo S I. Outcome after discontinuation of nucleot(s)ide analogues in chronic hepatitis B: relapse rate and associated factors. Ann Gastroenterol. 2015; 28(2):173-181.

Lau G K, Suri D, Liang R, et al. Resolution of Chronic Hepatitis B and Anti-HBs Seroconversion in Humans by Adoptive Transfer of Immunity to Hepatitis B Core Antigen. Gastroenterology 2002; 122:614-24.

Lacaille-Dubois, M and Wagner H, A review of the biological and pharmacological activities of saponins. Phytomedicine vol 2 pp 363-386 (1996).

Li J, Han Y, Jin K, et al. Dynamic changes of cytotoxic T lymphocytes (CTLs), natural killer (NK) cells, and natural killer T (NKT) cells in patients with acute hepatitis B infection. Virol J. 2011; 8:199.

Liang M, Ma S, Hu X, et al. Cellular immune responses in patients with hepatitis B surface antigen seroclearance induced by antiviral therapy. Virol J. 2011; 8:69.

Liaw Y F. Impact of therapy on the long-term outcome of chronic hepatitis B. Clin Liver Dis. 2013; 17:413-423.

Liaw Y F, Chu C M. Hepatitis B virus infection. Lancet 2009; 373, 582-592.

Lok A S, Pan C Q, Han S H, et al. Randomized phase II study of GS-4774 as a therapeutic vaccine in virally suppressed patients with chronic hepatitis B. J Hepatol. 2016; 65(3):509-16.

Lorin C, Vanloubbeeck Y, Baudart S, et al. Heterologous prime-boost regimens with a recombinant chimpanzee adenoviral vector and adjuvanted F4 protein elicit polyfunctional HIV-1-specific T-Cell responses in macaques. PLoS One. 2015; 10(4):e0122835.

Maini M, Gehring A, The role of innate immunity in the immunopathology and treatment of HBV infection. J Hepatol 2016; 64; S60-S70.

Martin P, Furman R R, Rutherford S, et al. Phase I study of the anti-CD74 monoclonal antibody milatuzumab (hLL1) in patients with previously treated B-cell lymphomas. Leuk Lymphoma. 2015; 12:1-6.

Mayr A, Stickl H, Muller H K, Danner K, Singer, H. The smallpox vaccination strain MVA: marker, genetic structure, experience gained with the parenteral vaccination and behavior in organisms with a debilitated defense mechanism. Zentralblatt fur Bakteriologie, Parasitenkunde, Infektionskrankheiten und Hygiene Erste Abteilung Originale Reihe B: Hygiene, Betriebshygiene, praventive Medizin. 1978; 167:375-90.

Mayr, A., Hochstein-Mintzel, V. & Stickl, H. (1975). Infection 3, 6-14.

McInnes K J, Smith L B, Hunger N I, Saunders P T, Andrew R, Walker B R. Deletion of the androgen receptor in adipose tissue in male mice elevates retinol binding protein 4 and reveals independent effects on visceral fat mass and on glucose homeostasis. Diabetes. 2012; 61(5):1072-81.

Mohamadnejad M, Tavangar S M, Sotoudeh M, et al. Histopathological Study of Chronic Hepatitis B: A Comparative Study of Ishak and METAVIR Scoring Systems. Int J Organ Transplant Med. 2010; 1(4):171-6.

Morawietz G, Ruehl-Fehlert C, Kittel B, et al. Revised guides for organ sampling and trimming in rats and mice—Part 3. A joint publication of the RITA and NACAD groups. Exp Toxicol Pathol. 2004; 55(6):433-49.

Neumann A U, Phillips S, Levine I, et al. Novel mechanism of antibodies to hepatitis B virus in blocking viral particle release from cells. Hepatology. 2010; 52(3):875-85.

Ott J J, Stevens G A, Groeger J, Wiersma S T. Global epidemiology of hepatitis B virus infection: new estimates of age-specific HBsAg seroprevalence and endemicity. Vaccine 2012; 30:2212-19.

Rammeh S, Khadra H B, Znaidi N S, et al. Inter-observes agreement of Ishak and Metavir scores in histological evaluation of chronic viral hepatitis B and C. Ann Biol Clin (Paris). 2014; 72(1):57-60.

Rehermann B, Nascimbeni M. Immunology of hepatitis B virus and hepatitis C virus infection. Nat Rev Immunol. 2005; 5(3):215-29.

Riedl P, Reiser M, Stifter K, Krieger J, Schirmbeck R. Differential presentation of endogenous and exogenous hepatitis B surface antigens influences priming of CD8(+) T cells in an epitope-specific manner. Eur J Immunol. 2014; 44(7):1981-91.

Ruehl-Fehlert C, Kittel B, Morawietz G, et al. Revised guides for organ sampling and trimming in rats and mice—part 1. Exp Toxicol Pathol. 2003; 55(2-3):91-106.

Spencer A J, Cottingham M G, Jenks J A, et al. Enhanced vaccine-induced CD8+ T cell responses to malaria antigen ME-TRAP by fusion to MHC class ii invariant chain. PLoS One. 2014; 9(6):e100538.

Terrault N A, Bzowej N H, Chang K-M, et al. AASLD Guidelines for Treatment of Chronic Hepatitis B. Hepatology. 2015; DOI:10.1002/hep.28156.

WHO Hepatitis D Fact Sheet, 23 Jul. 2018, https://www.who.int/news-room/fact-sheets/detail/hepatitis-d Accessed February 2019.

World Health Organization (WHO). Global Hepatitis report, 2017. http://apps.who.int/iris/bitstream/10665/255016/1/9789241565455-eng.pdf?ua=1. Accessed November 2017.

Yang F Q, Yu Y Y, Wang G Q, et al. A pilot randomized controlled trial of dual-plasmid HBV DNA vaccine mediated by in vivo electroporation in chronic hepatitis B patients under lamivudine chemotherapy. J Viral Hepat. 2012; 19:581-593.

Zoutendijk R, Hansen B E, van Vuuren A J, Boucher C A, Janssen H L. Serum HBsAg decline during long-term potent nucleos(t)ide analogue therapy for chronic hepatitis B and prediction of HBsAg loss. J Infect Dis. 2011; 204(3):415-8.

1. A method of treating chronic hepatitis B infection (CHB) and/or chronic hepatitis D infection (CHD) in a human, comprising the steps of:
a) administering to the human a composition comprising an antisense oligonucleotide (ASO) 10 to 30 nucleosides in length, targeted to a HBV nucleic acid (an HBV ASO);
b) administering to the human a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) administering to the human a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) administering to the human a composition comprising a recombinant hepatitis B surface antigen (HBs), a recombinant hepatitis B virus core antigen (HBc) and an adjuvant.

2. A method according to claim 1, wherein the steps b), c) and d) of the method are carried out sequentially, with step b) preceding step c) and step c) preceding step d).

3. A method according to claim 2, wherein step d) of the method is repeated.

4. A method according to claim 1 in which step a) is repeated.

5. A method according to claim 2 in which step a) is repeated prior to step b).

6. A method according to any preceding claim in which the period of time between each step is 1 week, 2 weeks, 4 weeks, 6 weeks 8 weeks, 12 weeks, 6 months or 12 months, for example 4 weeks or 8 weeks.

7. A method according to claim 1, wherein step d) is carried out concomitantly with step b) and/or with step c).

8. A method according to claim 7 in which step a) is repeated.

9. A method of treating chronic hepatitis B infection (CHB) and/or chronic hepatitis D (CHD) infection in a human, comprising the steps of:
a) administering to the human a composition comprising an antisense oligonucleotide (ASO) 10 to 30 nucleosides in length, targeted to a HBV nucleic acid (an HBV ASO);
b) administering to the human i) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc) and, concomitantly, ii) a composition comprising a recombinant hepatitis B surface antigen (HBs), a recombinant hepatitis B virus core antigen (HBc) and an adjuvant; and
c) administering to the human i) a composition comprising a Modified Vaccinia Virus Ankara (MVA) vector comprising a polynucleotide encoding a hepatitis B surface antigen (HBs) and a nucleic acid encoding a hepatitis B virus core antigen (HBc) and, concomitantly, a composition comprising a recombinant hepatitis B surface antigen (HBs), a recombinant hepatitis B virus core antigen (HBc) and an adjuvant.

10. A method according to claim 10 in which step a) is repeated and precedes step b), and step b) precedes step c).

11. A method according to any preceding claim, wherein the antisense oligonucleotide targeted to a HBV nucleic acid has the sequence GCAGAGGTGAAGCGAAGTGC.

12. A method according to any preceding claim, wherein the antisense oligonucleotide targeted to a HBV nucleic acid is a modified oligonucleotide “gapmer” consisting of 20 linked nucleosides in which each internucleoside linkage is a phosphorothioate linkage and each cytosine is a 5-methylcytosine, having the sequence GCAGAGGTGAAGCGAAGTGC consisting of a 5′ wing segment consisting of five linked nucleosides GCAGA each comprising a 2′-O-methoxyethyl sugar, followed by ten linked deoxynucleosides GGTGAAGCGA and a 3′ wing segment consisting of five linked nucleosides AGTGC each comprising a 2′-O-methoxyethyl sugar.

13. An immunogenic combination for use in a method of treating chronic hepatitis B infection (CHB) and/or chronic hepatitis D infection (CHD) in a human, the immunogenic combination comprising:
a) a composition comprising an antisense oligonucleotide (ASO) 10 to 30 nucleosides in length, targeted to a HBV nucleic acid (an HBV ASO);
b) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) a composition comprising a recombinant hepatitis B surface antigen (HBs), recombinant hepatitis B virus core antigen (HBc) and an adjuvant,
wherein the method comprises administering the compositions sequentially or concomitantly to the human.

14. The immunogenic combination according to claim 13, wherein the antisense oligonucleotide targeted to a HBV nucleic acid has the sequence GCAGAGGTGAAGCGAAGTGC.

15. The immunogenic combination according to claim 13 or 14 wherein the antisense oligonucleotide targeted to a HBV nucleic acid is a modified oligonucleotide “gapmer” consisting of 20 linked nucleosides in which each internucleoside linkage is a phosphorothioate linkage and each cytosine is a 5-methylcytosine, having the sequence GCAGAGGTGAAGCGAAGTGC consisting of a 5′ wing segment consisting of five linked nucleosides GCAGA each comprising a 2′-O-methoxyethyl sugar, followed by ten linked deoxynucleosides GGTGAAGCGA and a 3′ wing segment consisting of five linked nucleosides AGTGC each comprising a 2′-O-methoxyethyl sugar.

16. An immunogenic composition for use in a method of treating chronic hepatitis B infection (CHB) and/or chronic hepatitis D infection (CHD) in a human, the immunogenic composition comprising an antisense oligonucleotide 10 to 30 nucleosides in length, targeted to a HBV nucleic acid (an HBV ASO) and a replication-defective chimpanzee adenoviral (ChAd) vector comprising a polynucleotide encoding a hepatitis B surface antigen (HBs), a nucleic acid encoding a hepatitis B virus core antigen (HBc) and a nucleic acid encoding the human invariant chain (hIi) fused to the HBc, wherein the method comprises administration of the composition in a prime-boost regimen with at least one other immunogenic composition.

17. The immunogenic composition for use according to claim 16, further comprising one or more recombinant HBV protein antigens.

18. An immunogenic composition for use in a method of treating chronic hepatitis B infection (CHB) and/or chronic hepatitis D (CHD) infection in a human, the immunogenic composition comprising an antisense oligonucleotide 10 to 30 nucleosides in length, targeted to a HBV nucleic acid (an HBV ASO); and a Modified Vaccinia Virus Ankara (MVA) vector comprising a polynucleotide encoding a hepatitis B surface antigen (HBs) and a nucleic acid encoding a hepatitis B virus core antigen (HBc) wherein the method comprises administration of the composition in a prime-boost regimen with at least one other immunogenic composition.

19. The immunogenic composition for use according to claim 18 further comprising one or more recombinant HBV protein antigens.

20. An immunogenic composition for use in a method of treating chronic hepatitis B infection (CHB) and/or chronic hepatitis D (CHD) infection in a human, the immunogenic composition comprising an antisense oligonucleotide 10 to 30 nucleosides in length, targeted to a HBV nucleic acid (an HBV ASO); and a recombinant hepatitis B surface antigen (HBs), a C-terminal truncated recombinant hepatitis B virus core antigen (HBc) and an adjuvant containing MPL and QS-21, wherein the method comprises administration of the composition in a prime-boost regimen with at least one other immunogenic composition.

21. The immunogenic composition for use according to claim 20 in which the ratio of HBc to HBs in the composition is greater than 1.

22. The immunogenic composition for use according to claim 21 in which the ratio of HBc to HBs in the composition is 4:1.

23. The immunogenic composition for use according to any one of claims 20 to 22 further comprising one or more vectors encoding one or more HBV antigens.

24. The immunogenic composition for use according to any of claims 16 to 23, wherein the antisense oligonucleotide targeted to a HBV nucleic add has the sequence GCAGAGGTGAAGCGAAGTGC.

25. The immunogenic composition for use according to any of claims 16 to 24, wherein the antisense oligonucleotide targeted to a HBV nucleic acid is a modified oligonucleotide “gapmer” consisting of 20 linked nucleosides in which each internucleoside linkage is a phosphorothioate linkage and each cytosine is a 5-methylcytosine, having the sequence GCAGAGGTGAAGCGAAGTGC consisting of a 5′ wing segment consisting of five linked nucleosides GCAGA each comprising a 2′-O-methoxyethyl sugar, followed by ten linked deoxynucleosides GGTGAAGCGA and a 3′ wing segment consisting of five linked nucleosides AGTGC each comprising a 2′-O-methoxyethyl sugar.

26. The use of an immunogenic composition in the manufacture of a medicament for treating chronic hepatitis B infection (CHB) and/or chronic hepatitis D (CHD) infection in a human, the immunogenic composition comprising an antisense 10 to 30 nucleosides in length, targeted to a HBV nucleic acid (an HBV ASO) and a replication-defective chimpanzee adenoviral (ChAd) vector comprising a polynucleotide encoding a hepatitis B surface antigen (HBs), a nucleic acid encoding a hepatitis B virus core antigen (HBc) and a nucleic acid encoding the human invariant chain (hIi) fused to the nucleic acid encoding HBc, wherein the method of treating chronic hepatitis B infection and/or CHD infection comprises administration of the composition in a prime-boost regimen with at least one other immunogenic composition.

27. The use of an immunogenic composition in the manufacture of a medicament for treating chronic hepatitis B infection (CHB) and/or chronic hepatitis D (CHD) infection in a human, the immunogenic composition comprising an antisense 10 to 30 nucleosides in length, targeted to a HBV nucleic acid (an HBV ASO) and a Modified Vaccinia Virus Ankara (MVA) vector comprising a polynucleotide encoding a hepatitis B surface antigen (HBs) and a nucleic acid encoding a hepatitis B virus core antigen (HBc) wherein the method of treating chronic hepatitis B infection and/or CHD infection comprises administration of the composition in a prime-boost regimen with at least one other immunogenic composition.

28. The use of an immunogenic combination in the manufacture of a medicament for the treatment of chronic hepatitis B infection (CHB) and/or chronic hepatitis D (CHD) infection in a human, the immunogenic combination comprising:
a) an antisense oligonucleotide 10 to 30 nucleosides in length, targeted to a HBV nucleic acid (an HBV ASO);
b) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) a composition comprising a Modified Vaccinia Virus Ankara (MVA) vector comprising a polynucleotide encoding a hepatitis B surface antigen (HBs) and a nucleic add encoding a hepatitis B virus core antigen (HBc); and
d) a composition comprising a recombinant hepatitis B surface antigen (HBs), recombinant hepatitis B virus core antigen (HBc) and an adjuvant,
wherein the method of treating chronic hepatitis B infection and/or CHD infection comprises administering the compositions sequentially or concomitantly to the human.

29. The use of an immunogenic composition in the manufacture of a medicament according to any of claims 26 to 28, wherein the antisense oligonucleotide targeted to a HBV nucleic acid has the sequence GCAGAGGTGAAGCGAAGTGC.

30. The use of an immunogenic composition in the manufacture of a medicament according to any of claims 26 to 29, wherein the antisense oligonucleotide targeted to a HBV nucleic acid is a modified oligonucleotide “gapmer” consisting of 20 linked nucleosides in which each internucleoside linkage is a phosphorothioate linkage and each cytosine is a 5-methylcytosine, having the sequence GCAGAGGTGAAGCGAAGTGC consisting of a 5′ wing segment consisting of five linked nucleosides GCAGA each comprising a 2′-O-methoxyethyl sugar, followed by ten linked deoxynucleosides GGTGAAGCGA and a 3′ wing segment consisting of five linked nucleosides AGTGC each comprising a 2′-O-methoxyethyl sugar.

31. An immunogenic combination comprising:
a) a composition comprising an antisense oligonucleotide (ASO) 10 to 30 nucleosides in length, targeted to a HBV nucleic acid (an HBV ASO);
b) a composition comprising a replication-defective chimpanzee adenoviral (ChAd) vector comprising a polynucleotide encoding a hepatitis B surface antigen (HBs) and a nucleic acid encoding a hepatitis B virus core antigen (HBc);
c) a composition comprising a Modified Vaccinia Virus Ankara (MVA) vector comprising a polynucleotide encoding a hepatitis B surface antigen (HBs) and a nucleic acid encoding a hepatitis B virus core antigen (HBc); and
d) a composition comprising a recombinant hepatitis B surface antigen (HBs), recombinant hepatitis B virus core antigen (HBc) and an adjuvant.

32. The immunogenic combination according to claim 31, wherein the antisense oligonucleotide targeted to a HBV nucleic acid has the sequence GCAGAGGTGAAGCGAAGTGC.

33. The immunogenic combination according to claim 31 or 32, wherein the antisense oligonucleotide targeted to a HBV nucleic acid is a modified oligonucleotide “gapmer” consisting of 20 linked nucleosides in which each internucleoside linkage is a phosphorothioate linkage and each cytosine is a 5-methylcytosine, having the sequence GCAGAGGTGAAGCGAAGTGC consisting of a 5′ wing segment consisting of five linked nucleosides GCAGA each comprising a 2′-O-methoxyethyl sugar, followed by ten linked deoxynucleosides GGTGAAGCGA and a 3′ wing segment consisting of five linked nucleosides AGTGC each comprising a 2′-O-methoxyethyl sugar.


SEQUENCE LISTING (28 sequences)

SEQ ID NO: 1 - protein, 226 aa, Hepatitis B virus
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 2 - protein, 149 aa, Artificial Sequence
  Amino acid sequence of HBc truncate
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVV

SEQ ID NO: 3 - protein, 24 aa, Artificial Sequence
  Amino acid sequence of spacer incorporating 2A cleaving region of the foot and mouth disease virus
APVKQTLNFDLLKLAGDVESNPGP

SEQ ID NO: 4 - DNA, 72 bp, Artificial Sequence
  Nucleotide sequence encoding spacer incorporating 2A cleavage region of the foot and mouth disease virus
gcccctgtgaagcagaccctgaacttcgacctgctgaagctggccggcgacgtggagagcaatcccggccct

SEQ ID NO: 5 - protein, 435 aa, Artificial Sequence
  Amino acid sequence of HBc-2A-HBs
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQCAPVKQTLNFDLLKLAGDVESNPGPMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 6 - DNA, 1305 bp, Artificial Sequence
  Nucleotide sequence encoding HBc-2A-HBs
atggacatcgatccctacaaggaatttggcgccaccgtggagctgctgagcttcctgcccagcgacttcttccccagcgtgagggacctcctggacaccgccagcgccctgtacagggaggccctggaatctcccgagcactgcagcccacaccacaccgcactgaggcaggccatcctgtgctggggagagctgatgaccctcgccacctgggtgggcaacaacctggaggaccccgccagcagggacctggtggtgaactacgtcaacaccaacatgggcctgaagatcaggcagctgctgtggttccacatcagctgcctgaccttcggcagggagaccgtgctggagtacctggtgagcttcggcgtgtggatcaggacacctcccgcctacagaccccccaacgcccccatcctgagcaccctgcccgagaccacagtggtgaggaggagggacaggggcaggtcacccaggaggaggactccaagccccaggaggaggaggagccagagccccaggagaaggaggagccagagcagggagagccagtgcgcccctgtgaagcagaccctgaacttcgacctgctgaagctggccggcgacgtggagagcaatcccggccctatggagaacatcaccagcggcttcctgggccccctgctggtgctgcaggcaggcttcttcctgctgaccaggatcctgaccatcccccagagcctggacagctggtggaccagcctgaacttcctcggcgggagccccgtgtgcctgggccagaacagccagtctcccaccagcaatcacagccccaccagctgccccccaatctgtcctggctaccggtggatgtgcctgaggaggttcatcatcttcctgttcatcctgctcctgtgcctgatcttcctgctggtgctgctggactaccagggaatgctgccagtgtgtcccctgatccccggctcaaccaccactaacaccggcccctgcaaaacctgcaccacccccgctcagggcaacagcatgttcccaagctgctgctgcaccaagcccaccgacggcaactgcacctgcattcccatccccagcagctgggccttcgccaagtatctgtgggagtgggccagcgtgaggttcagctggctcagcctgctggtgcccttcgtccagtggtttgtgggcctgagccccaccgtgtggctgagcgccatctggatgatgtggtactggggccccagcctgtactccatcgtgagccccttcatccccctgctgcccattttcttctgcctgtgggtgtacatc

SEQ ID NO: 7 - protein, 229 aa, Artificial Sequence
  Amino acid sequence of hIi
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPKPVSKMRMATPLLMQALPMGALPQGPMQNATKYGNMTEDHVMHLLQNADPLKVYPPLKSFPENLRHLKNTMETIDWKVFESWMHHWLLFEMSRHSLEQKPTDAPPKESLELEDPSSGGVTKQDLGPVPM

SEQ ID NO: 8 - DNA, 696 bp, Artificial Sequence
  Nucleotide sequence encoding hIi
atgcacaggaggaggagcaggagctgcagggaggaccagaagcccgtgatggacgaccagcgcgacctgatcagcaacaacgagcagctgccaatgctgggcaggaggcccggagcacccgaaagcaagtgcagcaggggcgccctgtacaccggcttcagcatcctggtgaccctcctgctggccggccaggccaccaccgcctatttcctgtaccagcagcagggcaggctcgataagctgaccgtgacctcccagaacctgcagctggagaacctgaggatgaagctgcccaagccccccaagcccgtgagcaagatgaggatggccacccccctgctgatgcaggctctgcccatgggggccctgccccagggccccatgcagaacgccaccaaatacggcaacatgaccgaggaccacgtgatgcacctgctgcagaacgccgatcctctgaaggtgtacccacccctgaaaggcagcttccccgagaacctcaggcacctgaagaacaccatggagaccatcgactggaaggtgttcgagagctggatgcaccactggctgctgttcgagatgagccggcacagcctggagcagaagcccaccgacgcccctcccaaggagagcctcgagctcgaggacccaagcagcggcctgggcgtgaccaagcaggacctgggccccgtgcccatg

SEQ ID NO: 9 - protein, 628 aa, Artificial Sequence
  Amino acid sequence of hIi-HBc-2A-HBs
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPKPVSKMRMATPLLMQALPMGALPQGPMQNATKYGNMTEDHVMHLLQNADPLKVYPPLKSFPENLRHLKNTMETIDWKVFESWMHHWLLFEMSRHSLEQKPTDAPPKESLELEDPSSGGVTKQDLGPVPMMDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVAPVKQTLNFDLLKLAGDVESNPGPMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 10 - DNA, 1998 bp, Artificial Sequence
  Nucleotide sequence encoding hIi-HBc-2A-HBs
atgcacaggaggaggagcaggagctgcagggaggaccagaagcccgtgatggacgaccagcgcgacctgatcagcaacaacgagcagctgccaatgctgggcaggaggcccggagcacccgaaagcaagtgcagcaggggcgccctgtacaccggcttcagcatcctggtgaccctcctgctggccggccaggccaccaccgcctatttcctgtaccagcagcagggcaggctcgataagctgaccgtgacctcccagaacctgcagctggagaacctgaggatgaagctgcccaagccccccaagcccgtgagcaagatgaggatggccacccccctgctgatgcaggctctgcccatgggggccctgccccagggccccatgcagaacgccaccaaatacggcaacatgaccgaggaccacgtgatgcacctgctgcagaacgccgatcctctgaaggtgtacccacccctgaaaggcagcttccccgagaacctcaggcacctgaagaacaccatggagaccatcgactggaaggtgttcgagagctggatgcaccactggctgctgttcgagatgagccggcacagcctggagcagaagcccaccgacgcccctcccaaggagagcctcgagctcgaggacccaagcagcggcctgggcgtgaccaagcaggacctgggccccgtgcccatggacattgacccctacaaggagttcggcgccaccgtcgaactgctgagcttcctccccagcgacttcttcccctccgtgagggatctgctggacacagctagcgccctgtacagggaggccctggagagccccgagcactgcagcccccaccacacagccctgaggcaggccatcctctgttggggcgagctgatgaccctggccacctgggtgggcaataacctggaggaccccgccagcagggacctggtggtcaactacgtgaacaccaacatgggcctgaagatcaggcagctgctgtggttccacatcagctgcctgacctttggcagggagaccgtcctggagtacctggtgagcttcggcgtgtggatcaggactcccccagcctacaggccccctaacgcccccatcctgtctaccctgcccgagaccaccgtggtgaggaggagggacaggggcagaagccccaggagaaggacccctagccccaggaggaggaggagccagagccccaggaggaggaggagccagagccgggagagccagtgcgcccctgtgaagcagaccctgaacttcgacctgctgaagctggccggcgacgtggagagcaatcccggccctatggaaaacatcaccagcggcttcctgggccccctgctggtgctgcaggccggcttcttcctgctgaccaggatcctgaccattccccagtcactggacagctggtggaccagcctgaacttcctcggcgggagccccgtgtgcctgggccagaatagccagagccccaccagcaaccactctcccacttcctgcccccctatctgccccggctacaggtggatgtgcctgaggaggttcatcatcttcctgttcatcctgctgctgtgcctgatcttcctgctggtgctgctggactaccagggaatgctgcccgtgtgtcccctgatccccggaagcaccaccaccaacaccggcccctgcaagacctgcaccacccccgcccagggcaactctatgttccccagctgctgctgcaccaagcccaccgacggcaactgcacttgcattcccatccccagcagctgggccttcgccaaatatctgtgggagtgggccagcgtgaggtttagctggctgagcctgctggtgcccttcgtgcagtggtttgtgggcctgagccccaccgtgtggctgagcgccatctggatgatgtggtactggggcccctccctgtacagcatcgtgagccccttcatccccctcctgcccatcttcttctgcctgtgggtgtacatc

SEQ ID NO: 11 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 12 - protein, 231 aa, Artificial Sequence
  Amino acid sequence of hIi alternate variant
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVP

SEQ ID NO: 13 - DNA, 693 bp, Artificial Sequence
  Nucleotide sequence encoding hI alternate variant
atgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagagtcactggaactggaggacccgtcttctgggctgggtgtgaccaagcaggatctgggcccagtcccc

SEQ ID NO: 14 - DNA, 1998 bp, Artificial Sequence
  Alternative nucleic acid sequence of hIi-HBc- 2A-HBs
atgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagagtcactggaactggaggacccgtcttctgggctgggtgtgaccaagcaggatctgggcccagtccccatggacattgacccttataaagaatttggagctactgtggagttactctcgtttttgccttctgacttctttccttccgtcagagatctcctagacaccgcctcagctctgtatcgagaagccttagagtctcctgagcattgctcacctcaccatactgcactcaggcaagccattctctgctggggggaattgatgactctagctacctgggtgggtaataatttggaagatccagcatccagggatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgtgcccctgtgaagcagaccctgaacttcgacctgctgaagctggccggcgacgtggagagcaatcccggccctatggagaacatcacatcaggattcctaggacccctgctcgtgttacaggcggggtttttcttgttgacaagaatcctcacaataccgcagagtctagactcgtggtggacttctctcaattttctagggggatcacccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccaatacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatt

SEQ ID NO: 15 - protein, 666 aa, Artificial Sequence
  Alternative amino acid sequence of hIi-HBc-2A- HBs
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQCAPVKQTLNFDLLKLAGDVESNPGPMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 16 - DNA, 3182 bp, Hepatitis B virus
aattccacaacctttcaccaaactctgcaagatcccagagtgagaggcctgtatttccctgctggtggctccagttcaggagcagtaaaccctgttccgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcctgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaaccaccagcacgggaccatgccgaacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcctgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcatctagagacctagtagtcagttatgtcaacactaatatgggcctaaagttcaggcaactcttgtggtttcacatttcttgtctcacttttggaagagaaaccgttatagagtatttggtgtctttcggagtgtggattcgcactcctccagcttatagaccaccaaatgcccctatcctatcaacacttccggaaactactgttgttagacgacgaggcaggtcccctagaagaagaactccctcgcctcgcagacgaaggtctcaatcgccgcgtcgcagaagatctcaatctcgggaacctcaatgttagtattccttggactcataaggtggggaactttactggtctttattcttctactgtacctgtctttaatcctcattggaaaacaccatcttttcctaatatacatttacaccaagacattatcaaaaaatgtgaacagtttgtaggcccacttacagttaatgagaaaagaagattgcaattgattatgcctgctaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacacagcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagacaggaaggcagcctaccccgctgtctccacctttgagaaacactcatcctcaggccatgcagtgg

SEQ ID NO: 17 - DNA, 19 bp, Artificial Sequence
  Part of viral primer probe set RTS3370
cttggtcatgggccatcag

SEQ ID NO: 18 - DNA, 20 bp, Artificial Sequence
  Part of viral primer probe set RTS3370
cggctaggagttccgcagta

SEQ ID NO: 19 - DNA, 22 bp, Artificial Sequence
  Part of viral primer probe set RTS3370
tgcgtggaaccttttcggctcc

SEQ ID NO: 20 - DNA, 19 bp, Artificial Sequence
  Part of viral primer probe set RTS3371
ccaaaccttcggacggaaa

SEQ ID NO: 21 - DNA, 19 bp, Artificial Sequence
  Part of viral primer probe set RTS3371
tgaggcccactcccatagg

SEQ ID NO: 22 - DNA, 26 bp, Artificial Sequence
  Part of viral primer probe set RTS3371
cccatcatcctgggctttcggaaaat

SEQ ID NO: 23 - DNA, 24 bp, Artificial Sequence
  Part of viral primer probe set RTS3372
atcctatcaacacttccggaaact

SEQ ID NO: 24 - DNA, 16 bp, Artificial Sequence
  Part of viral primer probe set RTS3372
cgacgcggcgattgag

SEQ ID NO: 25 - DNA, 24 bp, Artificial Sequence
  Part of viral primer probe set RTS3372
aagaactccctcgcctcgcagacg

SEQ ID NO: 26 - DNA, 21 bp, Artificial Sequence
  Part of viral primer probe set RTS3373MGB
ccgaccttgaggcatacttca

SEQ ID NO: 27 - DNA, 27 bp, Artificial Sequence
  Part of viral primer probe set RTS3373MGB
aatttatgcctacagcctcctagtaca

SEQ ID NO: 28 - DNA, 20 bp, Artificial Sequence
  Part of viral primer probe set RTS3373MGB
ttaaagactgggaggagttg